(19) United States
(12) Patent Application Publication
(10) Pub. No.: US 2004/0005686 A1
(43) Pub. Date: Jan. 8, 2004

(54) Crystalline structure of human MAPKAP kinase-2

(75) Inventors: Ravi G. Kurumbail, St. Louis, MO (US); Jennifer L. Pawlitz, Wildwood, MO (US); Roderick A. Stegeman, Glen Carbon, IL (US); William C. Stallings, Wildwood, MO (US); Huey S. Shieh, St. Louis, MO (US); Robert J. Mourey, Ballwin, MO (US); Suzanne L. Bolten, Oakland, MO (US); Richard M. Broadus, St. Louis, MO (US)

Correspondence Address: SONNENSCHEIN NATH & ROSENTHAL, P.O. BOX 061080, WACKER DRIVE STATION, CHICAGO, IL, 60606-1080, US

(73) Assignee: Pharmacia Corporation

(21) Appl. No.: 10/116,649

(22) Filed: Apr. 4, 2002

Related U.S. Application Data
(60) Provisional application No. 60/362,380, filed on Mar. 7, 2002.

(51) Int. Cl. C12N 9/12; G06F 19/00; G01N 33/48; G01N 33/50; G01N 31/00

(52) U.S. Cl. 435/194; 702/19

(57) ABSTRACT

The crystal structure of human MAPKAP kinase-2 is described, including the high-resolution X-ray diffraction structure and atomic structure coordinates obtained therefrom. A method of crystallization of MK-2 involving the use of a crystallization additive and the specific empirical conditions involved in this crystallization method is also described. This method of crystallization allows a resolution of about 3 Angstroms to be achieved. The tertiary structure of this protein as determined by X-ray crystallography to a resolution of 3 Angstroms is shown. Also described are methods by which the atomic structural information obtained from the MK-2 crystals can be used to screen for, identify and/or design new drugs.

### CROSS-REFERENCE TO RELATED APPLICATIONS

The present application claims priority from a U.S. provisional application, Serial No. 60/362,380 filed Mar. 7, 2002, the entirety of which is hereby incorporated by reference.

### FIELD OF THE INVENTION

The present invention relates to the crystallization of human MAPKAP Kinase-2 (MK-2). More specifically, the invention relates to methods of crystallizing MK-2 and the unique empirical conditions involved in these crystallization methods. The present invention further relates to the crystal structure of human MK-2 itself, including the high-resolution X-ray diffraction structure and data obtained thereof. The MK-2 crystals of the invention and the atomic structural information obtained therefrom are useful for screening for, identifying and/or designing new drugs.

### BACKGROUND OF THE INVENTION

The response of cells to extracellular stimuli is mediated in part by a number of intracellular kinase and phosphatase enzymes. The mitogen-activated protein (MAP) kinases are participants in discrete signaling cascades, or pathways which function to convert extracellular stimuli into intracellular processes. One such mitogen-activated protein kinase (MAPK) pathway is the p38 signaling transduction pathway. The p38 signaling transduction pathway plays an essential role in regulating many cellular processes including inflammation, cell differentiation, cell growth and cell death.

The p38 MAPK pathway is potentially activated by a wide variety of stresses and cellular insults. These stresses and cellular insults include heat shock, UV radiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. (K. Ono, J. Han, Cellular Signalling 12 (2000) 1-13, 2.) Activation of the p38 pathway is involved in (1) production of proinflammatory cytokines such as TNF-α; (2) induction of enzymes such as COX-2, which controls connective tissue remodeling in pathological conditions; (3) expression of an intracellular enzyme such as iNOS, which plays an important role in the regulation of oxidation; (4) induction of adherent proteins such as VCAM-1 and many other inflammatory related molecules. Furthermore, the p38 pathway functions as a regulator in the proliferation and differentiation of cells of the immune system. Id. at 7.

p38 is an upstream kinase of mitogen-activated protein kinase-activated protein kinase-2 (MAPKAP kinase-2 or MK-2). (Freshney N W et al. J. Cell 1994;78:1039-49.) MK-2 is a protein which appears to be predominantly regulated by p38 in cells. Indeed, MAPKAP kinase-2 was the first substrate of p38α to be identified. For example, in vitro phosphorylation of MK-2 by p38α activates MK-2. The substrates which MAPKAP kinase-2 in turn act upon include heat shock protein 27, lymphocyte-specific protein 1 (LSP1), cAMP response element-binding protein (CREB), ATF1, SRF and tyrosine hydroxylase. The substrate of MAPKAP kinase-2 that has been best characterized is small heat shock protein 27 (hsp27). Supra at 6.

The role of the p38 pathway in inflammatory-related diseases has been studied in several animal models. SB203580 is a specific inhibitor of p38 in vivo and also has been shown to inhibit activation of MK-2. (Freshney N W et al. J.Cell 1994;78:1039-49; Rouse J, Cohen P, Trigon S, Morange M, Alonso-Llamazares A, Zamanillo D, Hunt T, Nebreda A R. Cell 1994;78:1027-37; Cuenda A, Dorow D S. Biochem J 1998;333:11-5.) Inhibition of p38 by SB203580 can reduce mortality in a murine model of endotoxin-induced shock and inhibit the development of mouse collagen-induced arthritis and rat adjuvant arthritis. (Badger A M, Bradbeer J N, Votta B, Lee J C, Adams J L, Griswold D E. J Pharmacol Exp Ther 1996;279:1453-61.) Another p38 inhibitor that has been utilized in an animal model that is believed to be more potent than SB203580 in its inhibitory effect on p38 is SB220025. A recent animal study has demonstrated that SB220025 caused a significant dose-dependent decrease in vascular density of granulomas in laboratory rats. (Jackson J R, Bolognese B, Hillegrass L, Kassis S, Adams J, Griswold D E, Winkler J D. J Pharmacol Exp Ther 1998;284:687-92.) The results of these animal studies have indicated that p38 or the components of the p38 pathway can be useful therapeutic targets for inflammatory disease.

Due to its integral role in the p38 signaling pathway, MK-2 has been used as a monitor for the level of activation in the pathway. MK-2 has been measured as a more convenient, albeit indirect, method of assessing p38 activation. However, so far research efforts have focused mainly on inhibiting p38 as a therapeutic strategy. These efforts have centered around two p38 inhibitors, the pyridinylimidazole inhibitor SKF 86002 and the 2,4,5 triaryl imidazole inhibitor SB203580. (John C. Lee et al., Inhibition of p38 MAP kinase as a therapeutic strategy”, Immunopharmacology 47 (2000), 185-201, 192.) Compounds possessing a similar structure have also been investigated as potential p38 inhibitors. Indeed, p38 MAP kinase's role in various disease states has been elucidated through the use of inhibitors. The discovery of information regarding the structural aspects of inhibitor/kinase interaction by techniques including X-ray crystallography and mutagenesis has made it possible to design second generation inhibitors with improved potency, selectivity and reduced undesirable side effects. Id. at 195.

MAPKAP kinase-2 has also been suggested as a focal point for regulating the inflammatory response. In “MAPKAP kinase 2 is essential for LPS-induced TNF-α biosynthesis” Alexey Kotlyarov et al. introduced a targeted mutation into the mouse MK-2 gene to investigate the function of MK-2 in vivo. Mice that lack MK-2 demonstrated enhanced stress resistance and were able to survive LPS-induced endotoxic shock. This phenomenon was shown to be a result of a reduction of approximately 90% in the production of TNF-α rather than being due to any change in signaling from the TNF receptor itself. The authors concluded that MK-2 regulates the biosynthesis of TNF-α at a post-transcriptional level and as such is an essential component in the inflammatory response. MAPKAP kinase-2 also has the potential advantage of being downstream from p38 in the p38 signaling transduction pathway and may as a focal point be effective in regulating the inflammatory response without affecting as many substrates as an enzyme further upstream in the signaling cascade would, such as p38 MAP kinase. By virtue of its downstream position in the p38 signaling transduction pathway, MAPKAP kinase-2 has the potential to yield inhibitors possessing similar advantages to those possessed by p38 MAP kinase inhibitors, namely, improved potency, selectivity and reduced undesirable side effects.

It would, therefore, be highly desirable to determine the structure of MK-2 in order to facilitate the identification and development of drugs for the treatment of inflammation, inflammatory diseases and related disorders. The three dimensional structure of MK-2 is expected to accelerate the drug discovery process of developing potent and selective inhibitors of MK-2.

### SUMMARY OF THE INVENTION

The present invention provides the MK-2 reagent that comprises amino acid residues 45-371 of human MK-2 for obtaining crystals of MK-2. The present invention further provides the crystal structure of human MK-2. The crystal structure of MK-2 was solved utilizing crystals of a complex of MK-2 formed from MK-2 grown in the presence of a non-hydrolysable ATP analog (AMP-PNP), a 13-residue inhibitor peptide (SC-83598) and MgCl₂. The X-ray crystallographic data were obtained from these crystals and the method of molecular replacement was then employed to determine the MK-2 crystal structure, using the program EPMR. The present invention thus provides a method of growing crystals by combining a solution of MK-2 polypeptide molecules with a precipitant solution containing a crystallization additive and allowing crystals of MK-2 to form using the method of vapor diffusion. Crystals formed with the use of certain crystallization additives enable the measurement of X-ray diffraction data to resolution of 3.0 Angstrom.

The present invention also provides the crystal structure of MK-2, including the mapping of the details of the ATP binding site. In a further embodiment of the invention methods are provided for screening for, identifying and/or designing new drugs using the crystal structure and data obtained thereof.

### BRIEF DESCRIPTION OF THE FIGURES

FIG. 1 is a ribbon drawing of the MK-2 crystal structure.

FIG. 2 is a stereo representation of the Cα rendering of the MK-2 complex.

FIG. 3 is an electron density map of the MK-2 crystal structure.

FIG. 4 is a sequence listing (SEQ ID No. 1) of the human MK-2 protein.

FIG. 5 is a sequence listing (SEQ ID No. 2) of the portion of the human MK-2 protein, amino acid numbers 45 to37 1, which were used for obtaining crystals of MK-2 for as discussed in this application.

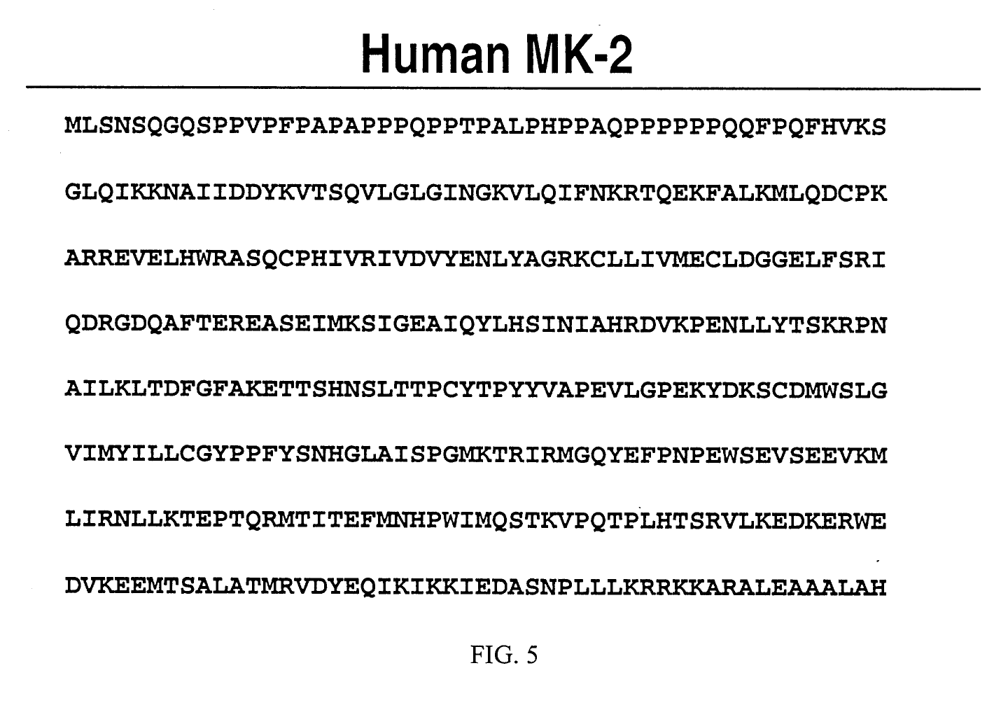

### DETAILED DESCRIPTION OF THE INVENTION

Crystallization and Structure Determination

The crystals from which the atomic structure coordinates of the invention are derived can be obtained by conventional means as are well-known in the art of protein crystallography, including batch, liquid bridge, dialysis, and vapor diffusion methods (see, e.g., McPherson, 1982, Preparation and Analysis of Protein Crystals, John Wiley, New York; McPherson, 1990, Eur. J. Biochem. 189:1-23.; Weber, 1991, Adv. Protein Chem. 41:1-36.). It is well known that the processes for obtaining crystals of particular proteins are individual to each protein. In a preferred embodiment, co-crystals are grown by the method of vapor diffusion involving hanging/sitting drops (McPherson, 1982, Preparation and Analysis of Protein Crystals, John Wiley, New York; McPherson, 1990, Eur. J. Biochem. 189:1-23.). In this method, the protein solution is allowed to equilibrate in a closed container with a larger aqueous reservoir having a precipitant concentration optimal for producing crystals. In general, approximately 2-5 μL of substantially pure polypeptide solution is mixed with an equal volume of reservoir solution, giving a precipitant concentration about half that required for crystallization. This solution is suspended as a droplet on a coverslip, which is then sealed on the top of the reservoir. The sealed container is allowed to stand, usually for about 2-6 weeks, until co-crystals grow.

Following this general procedure the co-crystals were grown by sitting drop vapor diffusion. Specifically, a protein solution was prepared consisting of 1.5-5 mg/mL MK-2(45-371) in 50 mM Tris at a pH of about 8 to 9 and containing around 15-50 mM NaCl, 2 mM DTT and 5% glycerol. This protein solution was mixed in a 1:1 ratio with a reservoir solution containing around 1.6-2.6M ammonium sulfate and 100 mM sodium acetate, the reservoir solution being at a pH of between around 4.2-5.4. Small bipyramidal or prism-shaped crystals appeared in the drops in 1-2 days and grew to as large as 0.4 mm×0.4 mm in about 1 to 3 weeks.

The process of crystallization was facilitated with the use of certain additives. These additives were selected from those additives that improve crystallization generally. Such additives can be divalent cations, non-volatile organic compounds, amphiphiles, ions, reducing agents, chelators, co-factors, carbohydrates, polyamines, linkers, polymers, solubilizing agents, dissociating agents, charotropes, detergents and salts. Many of the crystallization additives are salts. Examples of suitable crystallization additives are listed in Table 1 below.

TABLE 1

acetone
Anapoe ® C₁₃E₈
Anapoe ® X-114,
Cymal ®-1
C-HEGA-8 ™
C₁₂E₈
deoxy-BigChap
dichloromethane
dimethyl sulfoxide
1,4-dithio-DL-threitol (DTT)
EDTA sodium salt
ethanol
FOS-Choline ® 9
D(+) glucose
glycine
glycyl-glycyl-glycine
magnesium chloride
methanol
NAD⁺
n-dodecyl- -D-maltoside
n-hexadecyl- -D-maltoside
n-tetradecyl- -D-maltoside
n-tridecyl- -D-maltoside
1,3-propanediol
sodium fluoride
spermidine
spermidine-tetra-HCl
strontium chloride hexahydrate
tert-butanol
trimethylamine hydrochloride
TRITON X-100
urea
yttrium chloride hexahydrate

The additives of Table 1 are commercially available as crystallization Additive Screen kits I, II and III and Detergent Screens I, II, and III from Hampton Research Company, San Diego, Calif. Other additives, other additive screen kits and detergent screen kits can be used to identify additives which, when added to the aforementioned crystallization conditions, can facilitate crystallization. These additives can be added at a concentration of from between about 0 mM to about 150 mM. Preferably, the concentration of the additives is between about 3 mM to about 50 mM. More preferably, the concentration is around 5 mM to about 30 mM. Even more preferably, the concentration is between about 10 mM to about 20 mM.

The crystallization of molecules from solution is a reversible equilibrium process, and the kinetic and thermodynamic parameters are a function of the chemical and physical properties of the solvent system and solute of interest (McPherson, A., In: Preparation and Analysis of Protein Crystals, Wiley Interscience (1982); Weber, P. C., Adv. Protein Chem. 41:1-36 (1991)) 1991). Under supersaturating conditions, the system is driven toward equilibrium where the solute is partitioned between the soluble and solid phase instead of the unfolded and native states. The molecules in the crystalline phase pack in ordered and periodic three dimensional arrays that are energetically dominated by many of the same types of cohesive forces that are important for protein folding, i.e. van der Waals interactions, electrostatic interactions, hydrogen bonds, and covalent bonds (Moore, W. J., In: Physical Chemistry, 4th Ed., Prentice Hall, (1972), pp. 865-898).

Thus, in many ways protein crystallization can be viewed as a higher level variation of protein folding where whole molecules are packed to maximize cohesive energies instead of individual amino acid residues. Moreover, for both protein crystallization and protein folding, the composition of the solvent can make very important contributions to the extent of partitioning between the soluble (unfolded) and crystalline (native) forms. The cohesive interactions present in protein macromolecules and the role played by solvent in modulating these interactions for both protein folding and protein crystallization are complex and not fully understood at the present time. Without intending to be bound by any theory, it is believed that the crystallization additives participate in modulating these cohesive interactions in a manner that is advantageous to stability in the crystalline state.

The crystal structure was solved using crystals of MK-2 grown in the presence of a non-hydrolyzable ATP analog (AMP-PNP), a 13-residue inhibitor peptide (SC-83598) (KKKALLRQLGVAA) and MgCl₂. The structure of the inhibitor peptide SC-83598 is shown in Structure 1.

SC-83598 (inhibitor peptide)

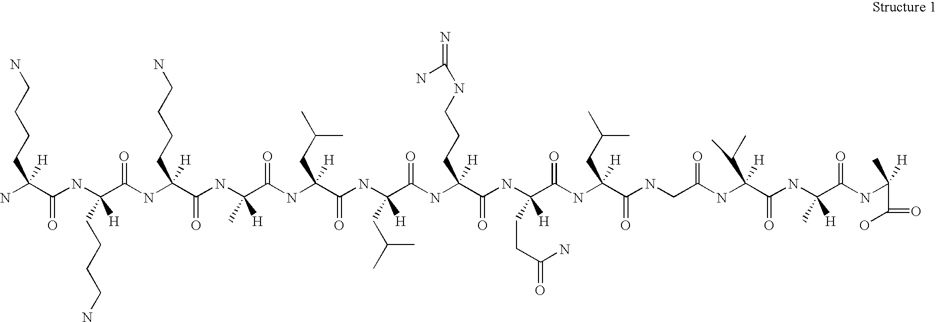

The structure of AMP-PNP is shown in Structure 2.

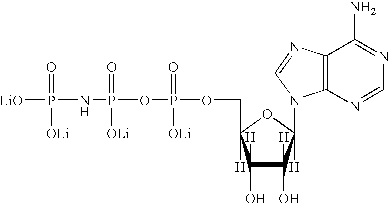

AMP-PNP (adenosine 5′-[β,gamma-imido] triphosphate tetralithium salt hydrate

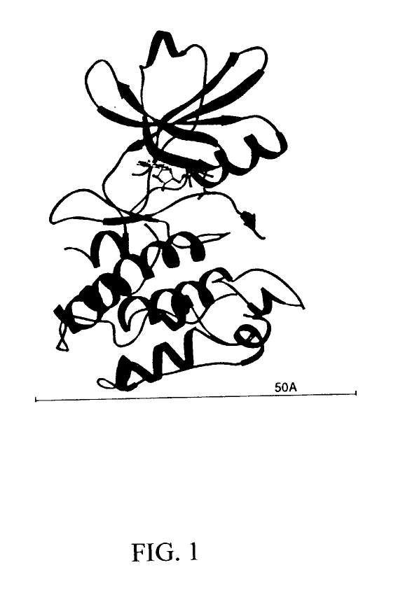

The MK-2 crystal structure that was obtained is shown in FIG. 1. The non-hydrolysable ATP analog (AMP-PNP) can be seen bound at the ATP site of MK-2 in the ribbon drawing in FIG. 1. Although residual electron density is visible at the site that is known to bind peptide substrate in protein kinases, the inhibitor peptide (SC83598) has not been modeled in the current structure.

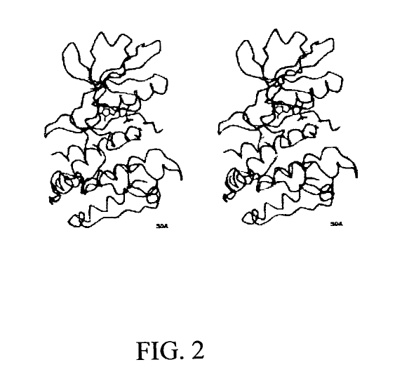

A stereo representation of the Cox rendering of the MK-2 complex is shown in FIG. 2. The AMP-PNP bound at the ATP site of MK-2 is also visible in this Calpha drawing of the MK-2 complex.

This complex of MK-2 was formed using enzyme/peptide/Mg²⁺/AMP-PNP molar ratios of 1:3:5:20, in a manner similar to that used in crystallizing a ternary complex of c-AMP-dependent protein kinase, as described by Zheng et al. in Crystal Structure of the Catalytic Subunit of cAMP-Dependent Protein Kinase Complexed with MgATP and Peptide Inhibitor, Biochemistry, 1993, Vol.32, No. 9, pages 2154-2161. The procedure used to form the ternary complex of c-AMP-dependent protein kinase is described specifically in the second paragraph of the first column of page 2155.

The dimensions of a unit cell of a crystal are defined by six numbers, the lengths of three unique edges, a, b, and c, and three unique angles α, β, and, γ. The type of unit cell that comprises a crystal is dependent on the value of these variables and the various symmetry elements that are present within the unit cell. The MK-2 crystal has a face-centered cubic lattice having the space group of F4₁32, and contain a single copy of the ternary complex in the asymmetic unit. The unit cell dimensions are about 254.8 (+/−2) Angstroms along the three edges. The unit cell contains 96 MK-2 molecules.

Of course, the processes for obtaining crystals of particular proteins are individual to each protein. Also, the presence of ligands can have a profound effect on the crystallization of a given protein. Thus, the processes for crystallizing the MK-2 protein, as with any protein, would change with a concomitant change in the MK-2 protein itself. For example, mutant proteins may crystallize under slightly different crystallization conditions compared to the wild-type protein, or under entirely new crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

X-Ray Diffraction

The diffraction data from X-ray crystallography is generally obtained as follows. When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hk1. The h index gives the number of parts into which the a edge of the unit cell is cut, the k index gives the number of parts into which the b edge of the unit cell is cut, and the 1 index gives the number of parts into which the c edge of the unit cell is cut by the set of hk1 planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hk1 indices, which correspond to the parallel planes from which the beam producing that spot was reflected. If the crystal is rotated about an axis perpendicular to the X-ray beam, a large number of reflections is recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume.

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal must be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising one or more cryoprotectants prior to exposure to liquid nitrogen, or the one or more cryoprotectants may be added to the crystallization solution. Data collection at liquid nitrogen temperatures may allow the collection of an entire dataset from one crystal.

Initial crystals of the MK-2 complex diffracted typically to about 4-5 Angstoms. This diffraction data was acquired at the Advanced Photon Source at Argonne National Laboratory. However, co-crystals grown in the presence of various additives allowed an improved resolution to be achieved. These additives are selected from those additives that improve crystallization generally. These preferred crystallization additives include deoxy-BigChap, n-hexadecyl-beta-D-maltoside, n-tridecyl-beta-D-maltoside, and Yttrium chloride hexahydrate. These preferred additives both facilitate the formation of crystals and allow an improved resolution to be achieved in X-ray diffraction. These preferred additives can be added at a concentration of from between about 0 mM to about 20 mM. Preferably, the concentration of the additives is between about 10 mM to about 20 mM.

Thus, preferred conditions for both crystallization and diffraction include concentrations of deoxy-BigChap, n-hexadecyl-beta-D-maltoside, Yttrium chloride hexahydrate or n-tridecyl-beta-D-maltoside between about 0 mM to about 20 mM, more preferably between about 10 mM to about 20 mM. Co-crystals of MK-2, AMP-PNP, magnesium, and SC-83598 grown in the presence of these additives can diffract to a resolution of better than 4-5 Angstroms. Preferably, the co-crystals of MK-2, AMP-PNP, magnesium, and SC-83598 grown in the presence of these additives can diffract to a resolution of better than 3.5 Angstroms. More preferably, the co-crystals of MK-2, AMP-PNP, magnesium, and SC-83598 grown in the presence of these additives can diffract to a resolution of between about 2.5 to about 3.3 Angstroms. This improved diffraction yielded the diffraction data summarized in Table 2.

TABLE 2
Summary of Diffraction Data From MK-2 Crystals

| Data Set | Crystal (1) | Crystal (2) | Crystal (3) |
|---|---|---|---|
| Resolution (A) | 40.0-3.0 | 40.0-3.3 | 40.0-3.3 |
| Rsymm | 7.7 | 8.6 | 7.6 |
| Completeness | 94.4 | 97.4 | 96.6 |
| Redundancy | 6.0 | 7.1 | 4.8 |
| Cell edge (A) | 254.0 | 253.5 | 253.5 |

The three dimensional (x, y, z) coordinates of MK-2 are shown below in Table 3 in the standard Protein Data Bank (PDB) format. (Bernstein F. C., et al. J. Mol. Biol. (1977) 122, 535).

TABLE 3

| | | | | | | | | | | |
|---|---|---|---|---|---|---|---|---|---|---|
| ATOM | 1 | CB | GLN | 45 | 102.406 | 264.041 | 78.035 | 1.00 | 100.00 | 6 |
| ATOM | 2 | CG | GLN | 45 | 103.925 | 264.125 | 78.221 | 1.00 | 100.00 | 6 |
| ATOM | 3 | CD | GLN | 45 | 104.542 | 265.347 | 77.549 | 1.00 | 100.00 | 6 |
| ATOM | 4 | OE1 | GLN | 45 | 103.923 | 265.981 | 76.687 | 1.00 | 100.00 | 8 |
| ATOM | 5 | NE2 | GLN | 45 | 105.769 | 265.683 | 77.946 | 1.00 | 97.48 | 7 |
| ATOM | 6 | C | GLN | 45 | 102.013 | 263.395 | 80.451 | 1.00 | 99.88 | 6 |
| ATOM | 7 | O | GLN | 45 | 102.125 | 262.501 | 81.293 | 1.00 | 100.00 | 8 |
| ATOM | 8 | N | GLN | 45 | 100.218 | 263.114 | 78.730 | 1.00 | 100.00 | 7 |
| ATOM | 9 | CA | GLN | 45 | 101.684 | 263.066 | 78.985 | 1.00 | 100.00 | 6 |
| ATOM | 10 | N | PHE | 46 | 102.123 | 264.685 | 80.746 | 1.00 | 95.46 | 7 |
| ATOM | 11 | CA | PHE | 46 | 102.439 | 265.167 | 82.082 | 1.00 | 89.46 | 6 |
| ATOM | 12 | CB | PHE | 46 | 103.465 | 266.301 | 81.948 | 1.00 | 87.16 | 6 |
| ATOM | 13 | CG | PHE | 46 | 104.122 | 266.716 | 83.238 | 1.00 | 74.67 | 6 |
| ATOM | 14 | CD1 | PHE | 46 | 105.202 | 266.009 | 83.742 | 1.00 | 67.95 | 6 |
| ATOM | 15 | CD2 | PHE | 46 | 103.722 | 267.873 | 83.895 | 1.00 | 67.67 | 6 |
| ATOM | 16 | CE1 | PHE | 46 | 105.872 | 266.457 | 84.876 | 1.00 | 66.39 | 6 |
| ATOM | 17 | CE2 | PHE | 46 | 104.391 | 268.322 | 85.029 | 1.00 | 59.60 | 6 |
| ATOM | 18 | CZ | PHE | 46 | 105.464 | 267.618 | 85.518 | 1.00 | 57.34 | 6 |
| ATOM | 19 | C | PHE | 46 | 101.180 | 265.661 | 82.790 | 1.00 | 86.96 | 6 |
| ATOM | 20 | O | PHE | 46 | 100.750 | 266.802 | 82.583 | 1.00 | 83.75 | 8 |
| ATOM | 21 | N | HIS | 47 | 100.547 | 264.775 | 83.558 | 1.00 | 86.89 | 7 |
| ATOM | 22 | CA | HIS | 47 | 99.336 | 265.152 | 84.286 | 1.00 | 87.76 | 6 |
| ATOM | 23 | CB | HIS | 47 | 98.081 | 264.344 | 83.878 | 1.00 | 90.07 | 6 |
| ATOM | 24 | CG | HIS | 47 | 98.312 | 262.879 | 83.676 | 1.00 | 92.87 | 6 |
| ATOM | 25 | CD2 | HIS | 47 | 97.520 | 261.927 | 83.126 | 1.00 | 94.34 | 6 |
| ATOM | 26 | ND1 | HIS | 47 | 99.474 | 262.238 | 84.051 | 1.00 | 99.02 | 7 |
| ATOM | 27 | CEl | HIS | 47 | 99.389 | 260.957 | 83.740 | 1.00 | 100.00 | 6 |
| ATOM | 28 | NE2 | HIS | 47 | 98.213 | 260.742 | 83.177 | 1.00 | 98.84 | 7 |
| ATOM | 29 | C | HIS | 47 | 99.535 | 265.156 | 85.791 | 1.00 | 78.29 | 6 |
| ATOM | 30 | O | HIS | 47 | 99.327 | 264.152 | 86.474 | 1.00 | 79.69 | 8 |
| ATOM | 31 | N | VAL | 48 | 99.998 | 266.305 | 86.274 | 1.00 | 66.71 | 7 |
| ATOM | 32 | CA | VAL | 48 | 100.257 | 266.532 | 87.680 | 1.00 | 55.57 | 6 |
| ATOM | 33 | CB | VAL | 48 | 101.402 | 267.540 | 87.896 | 1.00 | 55.61 | 6 |
| ATOM | 34 | CG1 | VAL | 48 | 102.703 | 266.958 | 87.434 | 1.00 | 63.49 | 6 |
| ATOM | 35 | CG2 | VAL | 48 | 101.120 | 268.829 | 87.152 | 1.00 | 52.37 | 6 |
| ATOM | 36 | C | VAL | 48 | 99.018 | 267.134 | 88.296 | 1.00 | 52.63 | 6 |
| ATOM | 37 | O | VAL | 48 | 98.098 | 267.558 | 87.604 | 1.00 | 49.40 | 8 |
| ATOM | 38 | N | LYS | 49 | 99.016 | 267.195 | 89.613 | 1.00 | 47.32 | 7 |
| ATOM | 39 | CA | LYS | 49 | 97.903 | 267.750 | 90.339 | 1.00 | 46.17 | 6 |
| ATOM | 40 | CB | LYS | 49 | 97.024 | 266.633 | 90.872 | 1.00 | 41.03 | 6 |
| ATOM | 41 | CG | LYS | 49 | 96.542 | 265.715 | 89.795 | 1.00 | 38.47 | 6 |
| ATOM | 42 | CD | LYS | 49 | 95.346 | 264.941 | 90.240 | 1.00 | 52.41 | 6 |
| ATOM | 43 | CE | LYS | 49 | 94.601 | 264.454 | 89.019 | 1.00 | 67.61 | 6 |
| ATOM | 44 | NZ | LYS | 49 | 94.186 | 265.601 | 88.160 | 1.00 | 75.30 | 7 |
| ATOM | 45 | C | LYS | 49 | 98.475 | 268.576 | 91.470 | 1.00 | 49.70 | 6 |
| ATOM | 46 | O | LYS | 49 | 99.553 | 268.289 | 91.971 | 1.00 | 53.57 | 8 |
| ATOM | 47 | N | SER | 50 | 97.738 | 269.591 | 91.885 | 1.00 | 51.51 | 7 |
| ATOM | 48 | CA | SER | 50 | 98.168 | 270.475 | 92.956 | 1.00 | 52.26 | 6 |
| ATOM | 49 | CB | SER | 50 | 97.079 | 271.519 | 93.208 | 1.00 | 56.64 | 6 |
| ATOM | 50 | OG | SER | 50 | 95.897 | 270.905 | 93.693 | 1.00 | 70.83 | 8 |
| ATOM | 51 | C | SER | 50 | 98.526 | 269.797 | 94.272 | 1.00 | 50.52 | 6 |
| ATOM | 52 | O | SER | 50 | 98.013 | 268.731 | 94.606 | 1.00 | 52.72 | 8 |
| ATOM | 53 | N | GLY | 51 | 99.423 | 270.430 | 95.013 | 1.00 | 43.31 | 7 |
| ATOM | 54 | CA | GLY | 51 | 99.808 | 269.910 | 96.306 | 1.00 | 46.43 | 6 |
| ATOM | 55 | C | GLY | 51 | 98.828 | 270.392 | 97.358 | 1.00 | 44.12 | 6 |
| ATOM | 56 | O | GLY | 51 | 97.862 | 271.083 | 97.031 | 1.00 | 46.55 | 8 |
| ATOM | 57 | N | LEU | 52 | 99.080 | 270.055 | 98.620 | 1.00 | 46.67 | 7 |
| ATOM | 58 | CA | LEU | 52 | 98.184 | 270.464 | 99.688 | 1.00 | 47.87 | 6 |
| ATOM | 59 | CB | LEU | 52 | 98.407 | 269.615 | 100.936 | 1.00 | 47.72 | 6 |
| ATOM | 60 | CG | LEU | 52 | 97.329 | 269.600 | 102.026 | 1.00 | 48.14 | 6 |
| ATOM | 61 | CD1 | LEU | 52 | 97.805 | 270.396 | 103.182 | 1.00 | 46.08 | 6 |
| ATOM | 62 | CD2 | LEU | 52 | 95.987 | 270.114 | 101.531 | 1.00 | 50.92 | 6 |
| ATOM | 63 | C | LEU | 52 | 98.296 | 271.952 | 100.003 | 1.00 | 51.25 | 6 |
| ATOM | 64 | O | LEU | 52 | 99.373 | 272.471 | 100.277 | 1.00 | 52.03 | 8 |
| ATOM | 65 | N | GLN | 53 | 97.150 | 272.621 | 99.937 | 1.00 | 52.39 | 7 |
| ATOM | 66 | CA | GLN | 53 | 97.028 | 274.050 | 100.198 | 1.00 | 55.58 | 6 |
| ATOM | 67 | CB | GLN | 53 | 96.029 | 274.673 | 99.201 | 1.00 | 73.54 | 6 |
| ATOM | 68 | CG | GLN | 53 | 95.030 | 273.678 | 98.466 | 1.00 | 86.15 | 6 |
| ATOM | 69 | CD | GLN | 53 | 94.026 | 272.909 | 99.373 | 1.00 | 86.10 | 6 |
| ATOM | 70 | OE1 | GLN | 53 | 93.195 | 273.506 | 100.080 | 1.00 | 82.19 | 8 |
| ATOM | 71 | NE2 | GLN | 53 | 94.075 | 271.579 | 99.299 | 1.00 | 76.86 | 7 |
| ATOM | 72 | C | GLN | 53 | 96.545 | 274.341 | 101.612 | 1.00 | 50.45 | 6 |
| ATOM | 73 | O | GLN | 53 | 95.340 | 274.300 | 101.876 | 1.00 | 52.43 | 8 |
| ATOM | 74 | N | ILE | 54 | 97.456 | 274.645 | 102.528 | 1.00 | 41.32 | 7 |
| ATOM | 75 | CA | ILE | 54 | 97.010 | 274.931 | 103.886 | 1.00 | 37.32 | 6 |
| ATOM | 76 | CB | ILE | 54 | 98.045 | 274.544 | 104.948 | 1.00 | 35.94 | 6 |
| ATOM | 77 | CG2 | ILE | 54 | 97.562 | 274.953 | 106.326 | 1.00 | 40.25 | 6 |
| ATOM | 78 | CG1 | ILE | 54 | 98.205 | 273.026 | 104.951 | 1.00 | 38.37 | 6 |
| ATOM | 79 | CD1 | ILE | 54 | 99.236 | 272.482 | 105.925 | 1.00 | 40.44 | 6 |
| ATOM | 80 | C | ILE | 54 | 96.590 | 276.378 | 104.035 | 1.00 | 34.42 | 6 |
| ATOM | 81 | O | ILE | 54 | 97.386 | 277.232 | 104.376 | 1.00 | 37.32 | 8 |
| ATOM | 82 | N | LYS | 55 | 95.316 | 276.627 | 103.765 | 1.00 | 35.43 | 7 |
| ATOM | 83 | CA | LYS | 55 | 94.714 | 277.955 | 103.850 | 1.00 | 38.05 | 6 |
| ATOM | 84 | CB | LYS | 55 | 93.238 | 277.872 | 103.420 | 1.00 | 40.98 | 6 |
| ATOM | 85 | CG | LYS | 55 | 92.991 | 277.575 | 101.941 | 1.00 | 47.96 | 6 |
| ATOM | 86 | CD | LYS | 55 | 91.513 | 277.259 | 101.681 | 1.00 | 55.91 | 6 |
| ATOM | 87 | CE | LYS | 55 | 91.084 | 277.555 | 100.236 | 1.00 | 61.30 | 6 |
| ATOM | 88 | NZ | LYS | 55 | 91.809 | 276.754 | 99.214 | 1.00 | 66.90 | 7 |
| ATOM | 89 | C | LYS | 55 | 94.786 | 278.503 | 105.283 | 1.00 | 37.65 | 6 |
| ATOM | 90 | O | LYS | 55 | 94.303 | 277.856 | 106.219 | 1.00 | 34.81 | 8 |
| ATOM | 91 | N | LYS | 56 | 95.385 | 279.683 | 105.456 | 1.00 | 34.35 | 7 |
| ATOM | 92 | CA | LYS | 56 | 95.487 | 280.266 | 106.789 | 1.00 | 31.71 | 6 |
| ATOM | 93 | CB | LYS | 56 | 96.803 | 281.023 | 107.004 | 1.00 | 32.85 | 6 |
| ATOM | 94 | CG | LYS | 56 | 98.060 | 280.388 | 106.414 | 1.00 | 46.33 | 6 |
| ATOM | 95 | CD | LYS | 56 | 98.249 | 278.923 | 106.766 | 1.00 | 46.90 | 6 |
| ATOM | 96 | CE | LYS | 56 | 98.461 | 278.723 | 108.238 | 1.00 | 58.39 | 6 |
| ATOM | 97 | NZ | LYS | 56 | 99.749 | 279.306 | 108.684 | 1.00 | 77.25 | 7 |
| ATOM | 98 | C | LYS | 56 | 94.316 | 281.181 | 107.149 | 1.00 | 31.86 | 6 |
| ATOM | 99 | O | LYS | 56 | 94.136 | 281.501 | 108.317 | 1.00 | 39.82 | 8 |
| ATOM | 100 | N | ASN | 57 | 93.501 | 281.584 | 106.177 | 1.00 | 25.32 | 7 |
| ATOM | 101 | CA | ASN | 57 | 92.358 | 282.462 | 106.473 | 1.00 | 29.61 | 6 |
| ATOM | 102 | CB | ASN | 57 | 91.746 | 282.991 | 105.191 | 1.00 | 30.38 | 6 |
| ATOM | 103 | CG | ASN | 57 | 91.062 | 281.921 | 104.396 | 1.00 | 30.96 | 6 |
| ATOM | 104 | OD1 | ASN | 57 | 89.852 | 281.729 | 104.524 | 1.00 | 36.24 | 8 |
| ATOM | 105 | ND2 | ASN | 57 | 91.821 | 281.226 | 103.549 | 1.00 | 29.49 | 7 |
| ATOM | 106 | C | ASN | 57 | 91.275 | 281.774 | 107.317 | 1.00 | 30.61 | 6 |
| ATOM | 107 | O | ASN | 57 | 91.218 | 280.547 | 107.378 | 1.00 | 33.98 | 8 |
| ATOM | 108 | N | ALA | 58 | 90.404 | 282.550 | 107.956 | 1.00 | 32.03 | 7 |
| ATOM | 109 | CA | ALA | 58 | 89.351 | 281.955 | 108.785 | 1.00 | 30.01 | 6 |
| ATOM | 110 | CB | ALA | 58 | 88.490 | 283.007 | 109.431 | 1.00 | 26.97 | 6 |
| ATOM | 111 | C | ALA | 58 | 88.516 | 281.086 | 107.913 | 1.00 | 32.48 | 6 |
| ATOM | 112 | O | ALA | 58 | 88.212 | 281.465 | 106.801 | 1.00 | 36.61 | 8 |
| ATOM | 113 | N | ILE | 59 | 88.153 | 279.915 | 108.410 | 1.00 | 35.25 | 7 |
| ATOM | 114 | CA | ILE | 59 | 87.345 | 278.997 | 107.633 | 1.00 | 36.12 | 6 |
| ATOM | 115 | CB | ILE | 59 | 87.232 | 277.658 | 108.349 | 1.00 | 33.16 | 6 |
| ATOM | 116 | CG2 | ILE | 59 | 86.502 | 277.850 | 109.647 | 1.00 | 29.32 | 6 |
| ATOM | 117 | CG1 | ILE | 59 | 86.528 | 276.639 | 107.448 | 1.00 | 34.35 | 6 |
| ATOM | 118 | CD1 | ILE | 59 | 86.239 | 275.315 | 108.122 | 1.00 | 40.00 | 6 |
| ATOM | 119 | C | ILE | 59 | 85.957 | 279.595 | 107.430 | 1.00 | 39.31 | 6 |
| ATOM | 120 | O | ILE | 59 | 85.263 | 279.312 | 106.450 | 1.00 | 43.48 | 8 |
| ATOM | 121 | N | ILE | 60 | 85.607 | 280.486 | 108.344 | 1.00 | 37.34 | 7 |
| ATOM | 122 | CA | ILE | 60 | 84.335 | 281.173 | 108.349 | 1.00 | 34.72 | 6 |
| ATOM | 123 | CB | ILE | 60 | 84.182 | 281.920 | 109.703 | 1.00 | 34.16 | 6 |
| ATOM | 124 | CG2 | ILE | 60 | 84.411 | 283.431 | 109.592 | 1.00 | 23.79 | 6 |
| ATOM | 125 | CG1 | ILE | 60 | 82.860 | 281.544 | 110.314 | 1.00 | 36.06 | 6 |
| ATOM | 126 | CD1 | ILE | 60 | 82.728 | 280.073 | 110.483 | 1.00 | 48.15 | 6 |
| ATOM | 127 | C | ILE | 60 | 84.088 | 282.072 | 107.139 | 1.00 | 41.20 | 6 |
| ATOM | 128 | O | ILE | 60 | 82.940 | 282.341 | 106.777 | 1.00 | 46.55 | 8 |
| ATOM | 129 | N | ASP | 61 | 85.165 | 282.515 | 106.501 | 1.00 | 37.84 | 7 |
| ATOM | 130 | CA | ASP | 61 | 85.069 | 283.376 | 105.332 | 1.00 | 35.17 | 6 |
| ATOM | 131 | CB | ASP | 61 | 86.431 | 284.007 | 105.010 | 1.00 | 32.16 | 6 |
| ATOM | 132 | CG | ASP | 61 | 86.998 | 284.841 | 106.175 | 1.00 | 57.04 | 6 |
| ATOM | 133 | OD1 | ASP | 61 | 86.321 | 284.987 | 107.228 | 1.00 | 61.36 | 8 |
| ATOM | 134 | OD2 | ASP | 61 | 88.139 | 285.352 | 106.037 | 1.00 | 58.09 | 8 |
| ATOM | 135 | C | ASP | 61 | 84.560 | 282.589 | 104.125 | 1.00 | 42.27 | 6 |
| ATOM | 136 | O | ASP | 61 | 83.848 | 283.135 | 103.283 | 1.00 | 50.87 | 8 |
| ATOM | 137 | N | ASP | 62 | 84.875 | 281.294 | 104.083 | 1.00 | 44.74 | 7 |
| ATOM | 138 | CA | ASP | 62 | 84.469 | 280.406 | 102.989 | 1.00 | 42.04 | 6 |
| ATOM | 139 | CB | ASP | 62 | 85.677 | 279.622 | 102.482 | 1.00 | 41.94 | 6 |
| ATOM | 140 | CG | ASP | 62 | 86.821 | 280.512 | 102.085 | 1.00 | 49.48 | 6 |
| ATOM | 141 | OD1 | ASP | 62 | 86.573 | 281.672 | 101.695 | 1.00 | 56.91 | 8 |
| ATOM | 142 | OD2 | ASP | 62 | 87.977 | 280.050 | 102.164 | 1.00 | 51.91 | 8 |
| ATOM | 143 | C | ASP | 62 | 83.311 | 279.422 | 103.181 | 1.00 | 43.99 | 6 |
| ATOM | 144 | O | ASP | 62 | 82.648 | 279.080 | 102.204 | 1.00 | 45.86 | 8 |
| ATOM | 145 | N | TYR | 63 | 83.063 | 278.968 | 104.408 | 1.00 | 39.85 | 7 |
| ATOM | 146 | CA | TYR | 63 | 81.976 | 278.017 | 104.666 | 1.00 | 40.47 | 6 |
| ATOM | 147 | CB | TYR | 63 | 82.563 | 276.684 | 105.131 | 1.00 | 35.58 | 6 |
| ATOM | 148 | CG | TYR | 63 | 83.251 | 275.868 | 104.068 | 1.00 | 30.21 | 6 |
| ATOM | 149 | CD1 | TYR | 63 | 84.588 | 276.115 | 103.701 | 1.00 | 21.95 | 6 |
| ATOM | 150 | CE1 | TYR | 63 | 85.199 | 275.380 | 102.696 | 1.00 | 14.53 | 6 |
| ATOM | 151 | CD2 | TYR | 63 | 82.558 | 274.866 | 103.408 | 1.00 | 24.00 | 6 |
| ATOM | 152 | CE2 | TYR | 63 | 83.153 | 274.126 | 102.408 | 1.00 | 30.67 | 6 |
| ATOM | 153 | CZ | TYR | 63 | 84.466 | 274.385 | 102.050 | 1.00 | 29.59 | 6 |
| ATOM | 154 | OH | TYR | 63 | 85.008 | 273.656 | 101.020 | 1.00 | 41.33 | 8 |
| ATOM | 155 | C | TYR | 63 | 81.024 | 278.491 | 105.744 | 1.00 | 43.02 | 6 |
| ATOM | 156 | O | TYR | 63 | 81.200 | 279.567 | 106.308 | 1.00 | 57.74 | 8 |
| ATOM | 157 | N | LYS | 64 | 80.000 | 277.692 | 106.019 | 1.00 | 43.49 | 7 |
| ATOM | 158 | CA | LYS | 64 | 79.032 | 278.037 | 107.055 | 1.00 | 45.33 | 6 |
| ATOM | 159 | CB | LYS | 64 | 77.632 | 278.333 | 106.506 | 1.00 | 48.55 | 6 |
| ATOM | 160 | CG | LYS | 64 | 76.730 | 278.937 | 107.587 | 1.00 | 58.11 | 6 |
| ATOM | 161 | CD | LYS | 64 | 75.253 | 279.082 | 107.204 | 1.00 | 68.53 | 6 |
| ATOM | 162 | CE | LYS | 64 | 74.421 | 279.540 | 108.437 | 1.00 | 79.27 | 6 |
| ATOM | 163 | NZ | LYS | 64 | 72.924 | 279.604 | 108.263 | 1.00 | 78.52 | 7 |
| ATOM | 164 | C | LYS | 64 | 78.997 | 276.838 | 108.002 | 1.00 | 48.19 | 6 |
| ATOM | 165 | O | LYS | 64 | 78.559 | 275.741 | 107.632 | 1.00 | 49.68 | 8 |
| ATOM | 166 | N | VAL | 65 | 79.532 | 277.031 | 109.202 | 1.00 | 42.62 | 7 |
| ATOM | 167 | CA | VAL | 65 | 79.561 | 275.965 | 110.182 | 1.00 | 43.03 | 6 |
| ATOM | 168 | CB | VAL | 65 | 80.686 | 276.179 | 111.189 | 1.00 | 45.24 | 6 |
| ATOM | 169 | CG1 | VAL | 65 | 80.776 | 274.992 | 112.150 | 1.00 | 47.57 | 6 |
| ATOM | 170 | CG2 | VAL | 65 | 81.998 | 276.386 | 110.449 | 1.00 | 43.02 | 6 |
| ATOM | 171 | C | VAL | 65 | 78.234 | 275.875 | 110.904 | 1.00 | 45.71 | 6 |
| ATOM | 172 | O | VAL | 65 | 77.753 | 276.857 | 111.439 | 1.00 | 52.95 | 8 |
| ATOM | 173 | N | THR | 66 | 77.629 | 274.695 | 110.865 | 1.00 | 49.19 | 7 |
| ATOM | 174 | CA | THR | 66 | 76.343 | 274.448 | 111.512 | 1.00 | 51.62 | 6 |
| ATOM | 175 | CB | THR | 66 | 75.368 | 273.706 | 110.559 | 1.00 | 48.24 | 6 |
| ATOM | 176 | OG1 | THR | 66 | 75.634 | 272.300 | 110.576 | 1.00 | 55.53 | 8 |
| ATOM | 177 | CG2 | THR | 66 | 75.547 | 274.197 | 109.138 | 1.00 | 44.65 | 6 |
| ATOM | 178 | C | THR | 66 | 76.569 | 273.607 | 112.765 | 1.00 | 52.90 | 6 |
| ATOM | 179 | O | THR | 66 | 77.543 | 272.851 | 112.834 | 1.00 | 52.91 | 8 |
| ATOM | 180 | N | SER | 67 | 75.661 | 273.708 | 113.734 | 1.00 | 60.50 | 7 |
| ATOM | 181 | CA | SER | 67 | 75.792 | 272.941 | 114.980 | 1.00 | 69.76 | 6 |
| ATOM | 182 | CB | SER | 67 | 75.119 | 273.666 | 116.154 | 1.00 | 72.74 | 6 |
| ATOM | 183 | OG | SER | 67 | 73.716 | 273.774 | 115.971 | 1.00 | 84.73 | 8 |
| ATOM | 184 | C | SER | 67 | 75.284 | 271.505 | 114.901 | 1.00 | 68.09 | 6 |
| ATOM | 185 | O | SER | 67 | 75.117 | 270.845 | 115.927 | 1.00 | 67.25 | 8 |
| ATOM | 186 | N | GLN | 68 | 75.012 | 271.041 | 113.685 | 1.00 | 69.13 | 7 |
| ATOM | 187 | CA | GLN | 68 | 74.526 | 269.685 | 113.462 | 1.00 | 69.93 | 6 |
| ATOM | 188 | CB | GLN | 68 | 73.831 | 269.575 | 112.106 | 1.00 | 75.31 | 6 |
| ATOM | 189 | CG | GLN | 68 | 73.348 | 268.162 | 111.786 | 1.00 | 85.40 | 6 |
| ATOM | 190 | CD | GLN | 68 | 72.759 | 268.046 | 110.381 | 1.00 | 93.11 | 6 |
| ATOM | 191 | OE1 | GLN | 68 | 71.723 | 268.645 | 110.098 | 1.00 | 98.35 | 8 |
| ATOM | 192 | NE2 | GLN | 68 | 73.365 | 267.307 | 109.471 | 1.00 | 95.21 | 7 |
| ATOM | 193 | C | GLN | 68 | 75.738 | 268.761 | 113.480 | 1.00 | 66.46 | 6 |
| ATOM | 194 | O | GLN | 68 | 76.820 | 269.117 | 112.995 | 1.00 | 68.83 | 8 |
| ATOM | 195 | N | VAL | 69 | 75.549 | 267.594 | 114.044 | 1.00 | 60.50 | 7 |
| ATOM | 196 | CA | VAL | 69 | 76.632 | 266.614 | 114.147 | 1.00 | 58.43 | 6 |
| ATOM | 197 | CB | VAL | 69 | 76.736 | 266.100 | 115.580 | 1.00 | 53.45 | 6 |
| ATOM | 198 | CG1 | VAL | 69 | 77.751 | 264.967 | 115.731 | 1.00 | 57.68 | 6 |
| ATOM | 199 | CG2 | VAL | 69 | 77.169 | 267.183 | 116.569 | 1.00 | 52.23 | 6 |
| ATOM | 200 | C | VAL | 69 | 76.376 | 265.442 | 113.206 | 1.00 | 58.19 | 6 |
| ATOM | 201 | O | VAL | 69 | 75.232 | 264.994 | 113.041 | 1.00 | 62.83 | 8 |
| ATOM | 202 | N | LEU | 70 | 77.463 | 264.983 | 112.617 | 1.00 | 59.48 | 7 |
| ATOM | 203 | CA | LEU | 70 | 77.439 | 263.854 | 111.680 | 1.00 | 55.76 | 6 |
| ATOM | 204 | CB | LEU | 70 | 78.297 | 264.148 | 110.446 | 1.00 | 53.10 | 6 |
| ATOM | 205 | CG | LEU | 70 | 77.685 | 265.176 | 109.489 | 1.00 | 48.51 | 6 |
| ATOM | 206 | CD1 | LEU | 70 | 78.745 | 265.926 | 108.674 | 1.00 | 50.87 | 6 |
| ATOM | 207 | CD2 | LEU | 70 | 76.739 | 264.556 | 108.457 | 1.00 | 50.63 | 6 |
| ATOM | 208 | C | LEU | 70 | 78.003 | 262.588 | 112.343 | 1.00 | 55.32 | 6 |
| ATOM | 209 | O | LEU | 70 | 77.786 | 261.469 | 111.864 | 1.00 | 55.85 | 8 |
| ATOM | 210 | N | GLY | 71 | 78.731 | 262.787 | 113.438 | 1.00 | 50.81 | 7 |
| ATOM | 211 | CA | GLY | 71 | 79.348 | 261.666 | 114.186 | 1.00 | 46.72 | 6 |
| ATOM | 212 | C | GLY | 71 | 80.312 | 262.184 | 115.266 | 1.00 | 47.78 | 6 |
| ATOM | 213 | O | GLY | 71 | 80.638 | 263.378 | 115.313 | 1.00 | 47.24 | 8 |
| ATOM | 214 | N | LEU | 72 | 80.750 | 261.254 | 116.112 | 1.00 | 47.63 | 7 |
| ATOM | 215 | CA | LEU | 72 | 81.669 | 261.566 | 117.226 | 1.00 | 50.20 | 6 |
| ATOM | 216 | CB | LEU | 72 | 80.993 | 261.319 | 118.579 | 1.00 | 49.87 | 6 |
| ATOM | 217 | CG | LEU | 72 | 79.689 | 262.096 | 118.760 | 1.00 | 54.65 | 6 |
| ATOM | 218 | CD1 | LEU | 72 | 78.458 | 261.284 | 118.354 | 1.00 | 54.07 | 6 |
| ATOM | 219 | CD2 | LEU | 72 | 79.443 | 262.526 | 120.209 | 1.00 | 58.26 | 6 |
| ATOM | 220 | C | LEU | 72 | 82.925 | 260.697 | 117.193 | 1.00 | 50.62 | 6 |
| ATOM | 221 | O | LEU | 72 | 82.939 | 259.650 | 116.548 | 1.00 | 50.74 | 8 |
| ATOM | 222 | N | GLY | 73 | 83.971 | 261.124 | 117.895 | 1.00 | 47.58 | 7 |
| ATOM | 223 | CA | GLY | 73 | 85.202 | 260.362 | 117.889 | 1.00 | 51.22 | 6 |
| ATOM | 224 | C | GLY | 73 | 86.359 | 260.959 | 118.672 | 1.00 | 60.34 | 6 |
| ATOM | 225 | O | GLY | 73 | 86.221 | 261.975 | 119.365 | 1.00 | 58.42 | 8 |
| ATOM | 226 | N | ILE | 74 | 87.522 | 260.326 | 118.514 | 1.00 | 65.56 | 7 |
| ATOM | 227 | CA | ILE | 74 | 88.759 | 260.703 | 119.199 | 1.00 | 66.96 | 6 |
| ATOM | 228 | CB | ILE | 74 | 89.969 | 259.885 | 118.684 | 1.00 | 70.55 | 6 |
| ATOM | 229 | CG2 | ILE | 74 | 89.881 | 258.435 | 119.159 | 1.00 | 67.80 | 6 |
| ATOM | 230 | CG1 | ILE | 74 | 90.060 | 260.001 | 117.159 | 1.00 | 74.63 | 6 |
| ATOM | 231 | CD1 | ILE | 74 | 91.332 | 259.424 | 116.560 | 1.00 | 83.87 | 6 |
| ATOM | 232 | C | ILE | 74 | 89.148 | 262.169 | 119.154 | 1.00 | 66.26 | 6 |
| ATOM | 233 | O | ILE | 74 | 90.018 | 262.598 | 119.909 | 1.00 | 68.57 | 8 |
| ATOM | 234 | N | ASN | 75 | 88.566 | 262.930 | 118.237 | 1.00 | 64.57 | 7 |
| ATOM | 235 | CA | ASN | 75 | 88.907 | 264.342 | 118.159 | 1.00 | 66.27 | 6 |
| ATOM | 236 | CB | ASN | 75 | 89.597 | 264.648 | 116.838 | 1.00 | 66.39 | 6 |
| ATOM | 237 | CG | ASN | 75 | 90.846 | 263.818 | 116.649 | 1.00 | 71.37 | 6 |
| ATOM | 238 | OD1 | ASN | 75 | 91.866 | 264.049 | 117.309 | 1.00 | 72.25 | 8 |
| ATOM | 239 | ND2 | ASN | 75 | 90.759 | 262.804 | 115.789 | 1.00 | 73.86 | 7 |
| ATOM | 240 | C | ASN | 75 | 87.695 | 265.233 | 118.384 | 1.00 | 66.72 | 6 |
| ATOM | 241 | O | ASN | 75 | 87.791 | 266.466 | 118.334 | 1.00 | 69.85 | 8 |
| ATOM | 242 | N | GLY | 76 | 86.567 | 264.592 | 118.680 | 1.00 | 60.95 | 7 |
| ATOM | 243 | CA | GLY | 76 | 85.346 | 265.319 | 118.927 | 1.00 | 56.44 | 6 |
| ATOM | 244 | C | GLY | 76 | 84.287 | 265.061 | 117.887 | 1.00 | 56.27 | 6 |
| ATOM | 245 | O | GLY | 76 | 84.266 | 264.018 | 117.238 | 1.00 | 55.10 | 8 |
| ATOM | 246 | N | LYS | 77 | 83.430 | 266.057 | 117.701 | 1.00 | 57.39 | 7 |
| ATOM | 247 | CA | LYS | 77 | 82.328 | 265.969 | 116.750 | 1.00 | 54.08 | 6 |
| ATOM | 248 | CB | LYS | 77 | 81.200 | 266.909 | 117.185 | 1.00 | 52.09 | 6 |
| ATOM | 249 | CG | LYS | 77 | 80.884 | 266.889 | 118.661 | 1.00 | 53.19 | 6 |
| ATOM | 250 | CD | LYS | 77 | 79.714 | 267.791 | 118.960 | 1.00 | 57.46 | 6 |
| ATOM | 251 | CE | LYS | 77 | 79.555 | 268.003 | 120.451 | 1.00 | 64.77 | 6 |
| ATOM | 252 | NZ | LYS | 77 | 78.465 | 268.963 | 120.763 | 1.00 | 70.29 | 7 |
| ATOM | 253 | C | LYS | 77 | 82.738 | 266.353 | 115.335 | 1.00 | 50.72 | 6 |
| ATOM | 254 | O | LYS | 77 | 83.542 | 267.257 | 115.149 | 1.00 | 54.53 | 8 |
| ATOM | 255 | N | VAL | 78 | 82.258 | 265.604 | 114.351 | 1.00 | 43.80 | 7 |
| ATOM | 256 | CA | VAL | 78 | 82.567 | 265.898 | 112.963 | 1.00 | 38.13 | 6 |
| ATOM | 257 | CB | VAL | 78 | 82.569 | 264.647 | 112.058 | 1.00 | 34.24 | 6 |
| ATOM | 258 | CG1 | VAL | 78 | 82.678 | 265.074 | 110.625 | 1.00 | 40.36 | 6 |
| ATOM | 259 | CG2 | VAL | 78 | 83.720 | 263.738 | 112.380 | 1.00 | 29.95 | 6 |
| ATOM | 260 | C | VAL | 78 | 81.330 | 266.711 | 112.631 | 1.00 | 41.66 | 6 |
| ATOM | 261 | O | VAL | 78 | 80.220 | 266.184 | 112.635 | 1.00 | 44.45 | 8 |
| ATOM | 262 | N | LEU | 79 | 81.511 | 267.998 | 112.379 | 1.00 | 40.18 | 7 |
| ATOM | 263 | CA | LEU | 79 | 80.391 | 268.881 | 112.059 | 1.00 | 41.65 | 6 |
| ATOM | 264 | CB | LEU | 79 | 80.707 | 270.262 | 112.637 | 1.00 | 38.15 | 6 |
| ATOM | 265 | CG | LEU | 79 | 80.110 | 270.716 | 113.976 | 1.00 | 31.75 | 6 |
| ATOM | 266 | CD1 | LEU | 79 | 79.719 | 269.572 | 114.881 | 1.00 | 27.67 | 6 |
| ATOM | 267 | CD2 | LEU | 79 | 81.103 | 271.625 | 114.637 | 1.00 | 30.45 | 6 |
| ATOM | 268 | C | LEU | 79 | 79.966 | 269.029 | 110.586 | 1.00 | 42.10 | 6 |
| ATOM | 269 | O | LEU | 79 | 80.795 | 268.928 | 109.695 | 1.00 | 43.31 | 8 |
| ATOM | 270 | N | GLN | 80 | 78.668 | 269.220 | 110.326 | 1.00 | 45.40 | 7 |
| ATOM | 271 | CA | GLN | 80 | 78.214 | 269.391 | 108.93 | 1.00 | 47.63 | 6 |
| ATOM | 272 | CB | GLN | 80 | 76.734 | 269.129 | 108.719 | 1.00 | 48.74 | 6 |
| ATOM | 273 | CG | GLN | 80 | 76.361 | 269.379 | 107.269 | 1.00 | 54.02 | 6 |
| ATOM | 274 | CD | GLN | 80 | 74.945 | 268.977 | 106.940 | 1.00 | 63.80 | 6 |
| ATOM | 275 | OE1 | GLN | 80 | 73.990 | 269.467 | 107.557 | 1.00 | 68.91 | 8 |
| ATOM | 276 | NE2 | GLN | 80 | 74.792 | 268.094 | 105.945 | 1.00 | 63.65 | 7 |
| ATOM | 277 | C | GLN | 80 | 78.513 | 270.847 | 108.645 | 1.00 | 47.13 | 6 |
| ATOM | 278 | O | GLN | 80 | 78.339 | 271.694 | 109.513 | 1.00 | 50.46 | 8 |
| ATOM | 279 | N | ILE | 81 | 78.816 | 271.162 | 107.399 | 1.00 | 42.64 | 7 |
| ATOM | 280 | CA | ILE | 81 | 79.145 | 272.524 | 107.064 | 1.00 | 40.48 | 6 |
| ATOM | 281 | CB | ILE | 81 | 80.693 | 272.635 | 107.273 | 1.00 | 38.74 | 6 |
| ATOM | 282 | CG2 | ILE | 81 | 81.474 | 272.717 | 105.983 | 1.00 | 31.42 | 6 |
| ATOM | 283 | CG1 | ILE | 81 | 81.012 | 273.723 | 108.273 | 1.00 | 48.96 | 6 |
| ATOM | 284 | CD1 | ILE | 81 | 82.474 | 273.835 | 108.567 | 1.00 | 55.74 | 6 |
| ATOM | 285 | C | ILE | 81 | 78.685 | 272.844 | 105.644 | 1.00 | 44.62 | 6 |
| ATOM | 286 | O | ILE | 81 | 78.404 | 271.925 | 104.885 | 1.00 | 46.52 | 8 |
| ATOM | 287 | N | PHE | 82 | 78.513 | 274.127 | 105.306 | 1.00 | 44.59 | 7 |
| ATOM | 288 | CA | PHE | 82 | 78.077 | 274.477 | 103.945 | 1.00 | 41.12 | 6 |
| ATOM | 289 | CB | PHE | 82 | 76.668 | 275.054 | 103.948 | 1.00 | 33.54 | 6 |
| ATOM | 290 | CG | PHE | 82 | 75.645 | 274.110 | 104.450 | 1.00 | 32.56 | 6 |
| ATOM | 291 | CD1 | PHE | 82 | 75.262 | 274.127 | 105.783 | 1.00 | 34.08 | 6 |
| ATOM | 292 | CD2 | PHE | 82 | 75.092 | 273.159 | 103.606 | 1.00 | 37.31 | 6 |
| ATOM | 293 | CE1 | PHE | 82 | 74.340 | 273.191 | 106.275 | 1.00 | 38.32 | 6 |
| ATOM | 294 | CE2 | PHE | 82 | 74.169 | 272.222 | 104.090 | 1.00 | 38.08 | 6 |
| ATOM | 295 | CZ | PHE | 82 | 73.796 | 272.242 | 105.427 | 1.00 | 34.81 | 6 |
| ATOM | 296 | C | PHE | 82 | 78.999 | 275.448 | 103.215 | 1.00 | 43.16 | 6 |
| ATOM | 297 | O | PHE | 82 | 79.437 | 276.445 | 103.782 | 1.00 | 46.44 | 8 |
| ATOM | 298 | N | ASN | 83 | 79.336 | 275.128 | 101.972 | 1.00 | 41.27 | 7 |
| ATOM | 299 | CA | ASN | 83 | 80.200 | 275.990 | 101.182 | 1.00 | 42.24 | 6 |
| ATOM | 300 | CB | ASN | 83 | 80.743 | 275.208 | 99.986 | 1.00 | 43.94 | 6 |
| ATOM | 301 | CG | ASN | 83 | 81.437 | 276.093 | 98.952 | 1.00 | 41.93 | 6 |
| ATOM | 302 | OD1 | ASN | 83 | 80.787 | 276.658 | 98.081 | 1.00 | 41.79 | 8 |
| ATOM | 303 | ND2 | ASN | 83 | 82.757 | 276.167 | 99.012 | 1.00 | 35.30 | 7 |
| ATOM | 304 | C | ASN | 83 | 79.336 | 277.153 | 100.702 | 1.00 | 43.66 | 6 |
| ATOM | 305 | O | ASN | 83 | 78.489 | 276.971 | 99.847 | 1.00 | 48.05 | 8 |
| ATOM | 306 | N | LYS | 84 | 79.546 | 278.344 | 101.249 | 1.00 | 42.83 | 7 |
| ATOM | 307 | CA | LYS | 84 | 78.770 | 279.532 | 100.863 | 1.00 | 38.88 | 6 |
| ATOM | 308 | CB | LYS | 84 | 79.491 | 280.783 | 101.353 | 1.00 | 32.85 | 6 |
| ATOM | 309 | CG | LYS | 84 | 79.523 | 280.909 | 102.865 | 1.00 | 27.55 | 6 |
| ATOM | 310 | CD | LYS | 84 | 80.393 | 282.038 | 103.280 | 1.00 | 32.58 | 6 |
| ATOM | 311 | CE | LYS | 84 | 80.051 | 282.486 | 104.670 | 1.00 | 36.92 | 6 |
| ATOM | 312 | NZ | LYS | 84 | 81.018 | 283.559 | 105.010 | 1.00 | 51.72 | 7 |
| ATOM | 313 | C | LYS | 84 | 78.513 | 279.672 | 99.365 | 1.00 | 39.20 | 6 |
| ATOM | 314 | O | LYS | 84 | 77.379 | 279.709 | 98.908 | 1.00 | 42.34 | 8 |
| ATOM | 315 | N | ARG | 85 | 79.597 | 279.713 | 98.612 | 1.00 | 41.62 | 7 |
| ATOM | 316 | CA | ARG | 85 | 79.579 | 279.847 | 97.162 | 1.00 | 44.84 | 6 |
| ATOM | 317 | CB | ARG | 85 | 81.011 | 279.668 | 96.681 | 1.00 | 52.98 | 6 |
| ATOM | 318 | CG | ARG | 85 | 81.339 | 279.950 | 95.236 | 1.00 | 57.19 | 6 |
| ATOM | 319 | CD | ARG | 85 | 82.801 | 279.486 | 94.986 | 1.00 | 72.19 | 6 |
| ATOM | 320 | NE | ARG | 85 | 83.740 | 279.926 | 96.047 | 1.00 | 82.00 | 7 |
| ATOM | 321 | CZ | ARG | 85 | 84.383 | 279.114 | 96.900 | 1.00 | 81.32 | 6 |
| ATOM | 322 | NH1 | ARG | 85 | 84.219 | 277.794 | 96.835 | 1.00 | 85.38 | 7 |
| ATOM | 323 | NH2 | ARG | 85 | 85.151 | 279.626 | 97.859 | 1.00 | 75.16 | 7 |
| ATOM | 324 | C | ARG | 85 | 78.648 | 278.909 | 96.383 | 1.00 | 46.34 | 6 |
| ATOM | 325 | O | ARG | 85 | 77.944 | 279.363 | 95.490 | 1.00 | 54.59 | 8 |
| ATOM | 326 | N | THR | 86 | 78.594 | 277.628 | 96.742 | 1.00 | 40.87 | 7 |
| ATOM | 327 | CA | THR | 86 | 77.737 | 276.683 | 96.023 | 1.00 | 38.65 | 6 |
| ATOM | 328 | CB | THR | 86 | 78.548 | 275.502 | 95.500 | 1.00 | 35.15 | 6 |
| ATOM | 329 | OG1 | THR | 86 | 78.997 | 274.724 | 96.611 | 1.00 | 44.88 | 8 |
| ATOM | 330 | CG2 | THR | 86 | 79.752 | 275.966 | 94.751 | 1.00 | 33.53 | 6 |
| ATOM | 331 | C | THR | 86 | 76.612 | 276.078 | 96.838 | 1.00 | 40.46 | 6 |
| ATOM | 332 | O | THR | 86 | 75.726 | 275.428 | 96.291 | 1.00 | 44.60 | 8 |
| ATOM | 333 | N | GLN | 87 | 76.680 | 276.266 | 98.145 | 1.00 | 38.56 | 7 |
| ATOM | 334 | CA | GLN | 87 | 75.694 | 275.747 | 99.076 | 1.00 | 39.20 | 6 |
| ATOM | 335 | CB | GLN | 87 | 74.318 | 276.303 | 98.773 | 1.00 | 32.20 | 6 |
| ATOM | 336 | CG | GLN | 87 | 74.199 | 277.741 | 99.114 | 1.00 | 42.79 | 6 |
| ATOM | 337 | CD | GLN | 87 | 74.108 | 277.971 | 100.591 | 1.00 | 50.66 | 6 |
| ATOM | 338 | OE1 | GLN | 87 | 73.171 | 277.493 | 101.227 | 1.00 | 55.20 | 8 |
| ATOM | 339 | NE2 | GLN | 87 | 75.071 | 278.713 | 101.156 | 1.00 | 51.42 | 7 |
| ATOM | 340 | C | GLN | 87 | 75.653 | 274.223 | 99.207 | 1.00 | 45.59 | 6 |
| ATOM | 341 | O | GLN | 87 | 74.636 | 273.661 | 99.630 | 1.00 | 49.90 | 8 |
| ATOM | 342 | N | GLU | 88 | 76.751 | 273.555 | 98.844 | 1.00 | 48.14 | 7 |
| ATOM | 343 | CA | GLU | 88 | 76.826 | 272.096 | 98.942 | 1.00 | 52.41 | 6 |
| ATOM | 344 | CB | GLU | 88 | 77.826 | 271.536 | 97.940 | 1.00 | 56.92 | 6 |
| ATOM | 345 | CG | GLU | 88 | 77.384 | 271.716 | 96.500 | 1.00 | 72.33 | 6 |
| ATOM | 346 | CD | GLU | 88 | 78.506 | 271.494 | 95.495 | 1.00 | 78.20 | 6 |
| ATOM | 347 | OE1 | GLU | 88 | 78.279 | 270.775 | 94.491 | 1.00 | 78.58 | 8 |
| ATOM | 348 | OE2 | GLU | 88 | 79.611 | 272.057 | 95.702 | 1.00 | 80.85 | 8 |
| ATOM | 349 | C | GLU | 88 | 77.222 | 271.707 | 100.372 | 1.00 | 52.55 | 6 |
| ATOM | 350 | O | GLU | 88 | 77.835 | 272.500 | 101.095 | 1.00 | 53.86 | 8 |
| ATOM | 351 | N | LYS | 89 | 76.862 | 270.493 | 100.779 | 1.00 | 49.27 | 7 |
| ATOM | 352 | CA | LYS | 89 | 77.174 | 270.010 | 102.123 | 1.00 | 48.29 | 6 |
| ATOM | 353 | CB | LYS | 89 | 76.097 | 269.036 | 102.638 | 1.00 | 52.03 | 6 |
| ATOM | 354 | CG | LYS | 89 | 75.125 | 268.494 | 101.564 | 1.00 | 72.16 | 6 |
| ATOM | 355 | CD | LYS | 89 | 74.065 | 269.559 | 101.138 | 1.00 | 83.35 | 6 |
| ATOM | 356 | CE | LYS | 89 | 73.454 | 269.295 | 99.754 | 1.00 | 80.24 | 6 |
| ATOM | 357 | NZ | LYS | 89 | 74.505 | 269.274 | 98.688 | 1.00 | 83.18 | 7 |
| ATOM | 358 | C | LYS | 89 | 78.544 | 269.368 | 102.220 | 1.00 | 47.72 | 6 |
| ATOM | 359 | O | LYS | 89 | 78.964 | 268.640 | 101.326 | 1.00 | 54.20 | 8 |
| ATOM | 360 | N | PHE | 90 | 79.246 | 269.660 | 103.309 | 1.00 | 45.18 | 7 |
| ATOM | 361 | CA | PHE | 90 | 80.572 | 269.122 | 103.554 | 1.00 | 41.92 | 6 |
| ATOM | 362 | CB | PHE | 90 | 81.586 | 270.139 | 103.133 | 1.00 | 36.66 | 6 |
| ATOM | 363 | CG | PHE | 90 | 81.730 | 270.257 | 101.655 | 1.00 | 39.45 | 6 |
| ATOM | 364 | CD1 | PHE | 90 | 82.324 | 269.244 | 100.926 | 1.00 | 40.56 | 6 |
| ATOM | 365 | CD2 | PHE | 90 | 81.379 | 271.422 | 101.001 | 1.00 | 40.83 | 6 |
| ATOM | 366 | CE1 | PHE | 90 | 82.571 | 269.402 | 99.571 | 1.00 | 43.06 | 6 |
| ATOM | 367 | CE2 | PHE | 90 | 81.629 | 271.582 | 99.644 | 1.00 | 38.17 | 6 |
| ATOM | 368 | CZ | PHE | 90 | 82.226 | 270.580 | 98.933 | 1.00 | 35.41 | 6 |
| ATOM | 369 | C | PHE | 90 | 80.755 | 268.764 | 105.024 | 1.00 | 44.84 | 6 |
| ATOM | 370 | O | PHE | 90 | 79.888 | 269.073 | 105.831 | 1.00 | 51.86 | 8 |
| ATOM | 371 | N | ALA | 91 | 81.842 | 268.072 | 105.373 | 1.00 | 39.95 | 7 |
| ATOM | 372 | CA | ALA | 91 | 82.076 | 267.699 | 106.771 | 1.00 | 31.48 | 6 |
| ATOM | 373 | CB | ALA | 91 | 82.148 | 266.222 | 106.931 | 1.00 | 34.87 | 6 |
| ATOM | 374 | C | ALA | 91 | 83.348 | 268.326 | 107.261 | 1.00 | 33.54 | 6 |
| ATOM | 375 | O | ALA | 91 | 84.350 | 268.344 | 106.559 | 1.00 | 42.48 | 8 |
| ATOM | 376 | N | LEU | 92 | 83.293 | 268.840 | 108.479 | 1.00 | 33.04 | 7 |
| ATOM | 377 | CA | LEU | 92 | 84.407 | 269.498 | 109.141 | 1.00 | 28.82 | 6 |
| ATOM | 378 | CB | LEU | 92 | 83.920 | 270.805 | 109.723 | 1.00 | 23.23 | 6 |
| ATOM | 379 | CG | LEU | 92 | 84.962 | 271.643 | 110.423 | 1.00 | 24.07 | 6 |
| ATOM | 380 | CD1 | LEU | 92 | 85.909 | 272.134 | 109.369 | 1.00 | 33.26 | 6 |
| ATOM | 381 | CD2 | LEU | 92 | 84.338 | 272.814 | 111.171 | 1.00 | 25.16 | 6 |
| ATOM | 382 | C | LEU | 92 | 84.921 | 268.649 | 110.289 | 1.00 | 40.27 | 6 |
| ATOM | 383 | O | LEU | 92 | 84.157 | 268.182 | 111.145 | 1.00 | 44.45 | 8 |
| ATOM | 384 | N | LYS | 93 | 86.235 | 268.482 | 110.311 | 1.00 | 45.27 | 7 |
| ATOM | 385 | CA | LYS | 93 | 86.930 | 267.707 | 111.332 | 1.00 | 41.65 | 6 |
| ATOM | 386 | CB | LYS | 93 | 87.596 | 266.501 | 110.675 | 1.00 | 37.36 | 6 |
| ATOM | 387 | CG | LYS | 93 | 88.303 | 265.598 | 111.620 | 1.00 | 33.77 | 6 |
| ATOM | 388 | CD | LYS | 93 | 88.724 | 264.365 | 110.885 | 1.00 | 36.49 | 6 |
| ATOM | 389 | CE | LYS | 93 | 88.861 | 263.201 | 111.850 | 1.00 | 42.11 | 6 |
| ATOM | 390 | NZ | LYS | 93 | 88.934 | 261.912 | 111.132 | 1.00 | 38.11 | 7 |
| ATOM | 391 | C | LYS | 93 | 87.974 | 268.654 | 111.939 | 1.00 | 43.64 | 6 |
| ATOM | 392 | O | LYS | 93 | 88.759 | 269.267 | 111.217 | 1.00 | 45.73 | 8 |
| ATOM | 393 | N | MET | 94 | 87.951 | 268.814 | 113.253 | 1.00 | 41.61 | 7 |
| ATOM | 394 | CA | MET | 94 | 88.899 | 269.695 | 113.905 | 1.00 | 47.32 | 6 |
| ATOM | 395 | CB | MET | 94 | 88.136 | 270.627 | 114.831 | 1.00 | 56.18 | 6 |
| ATOM | 396 | CG | MET | 94 | 86.914 | 271.277 | 114.187 | 1.00 | 69.87 | 6 |
| ATOM | 397 | SD | MET | 94 | 86.015 | 272.356 | 115.325 | 1.00 | 81.88 | 16 |
| ATOM | 398 | CE | MET | 94 | 87.465 | 273.270 | 116.131 | 1.00 | 71.12 | 6 |
| ATOM | 399 | C | MET | 94 | 89.948 | 268.945 | 114.710 | 1.00 | 50.04 | 6 |
| ATOM | 400 | O | MET | 94 | 89.618 | 267.997 | 115.415 | 1.00 | 57.12 | 8 |
| ATOM | 401 | N | LEU | 95 | 91.213 | 269.350 | 114.603 | 1.00 | 50.63 | 7 |
| ATOM | 402 | CA | LEU | 95 | 92.294 | 268.688 | 115.354 | 1.00 | 52.04 | 6 |
| ATOM | 403 | CB | LEU | 95 | 93.269 | 267.930 | 114.446 | 1.00 | 46.71 | 6 |
| ATOM | 404 | CG | LEU | 95 | 92.911 | 266.734 | 113.573 | 1.00 | 36.39 | 6 |
| ATOM | 405 | CD1 | LEU | 95 | 91.531 | 266.159 | 113.868 | 1.00 | 32.69 | 6 |
| ATOM | 406 | CD2 | LEU | 95 | 93.007 | 267.222 | 112.158 | 1.00 | 40.02 | 6 |
| ATOM | 407 | C | LEU | 95 | 93.084 | 269.726 | 116.132 | 1.00 | 56.47 | 6 |
| ATOM | 408 | O | LEU | 95 | 92.912 | 270.919 | 115.914 | 1.00 | 60.28 | 8 |
| ATOM | 409 | N | GLN | 96 | 93.986 | 269.269 | 116.996 | 1.00 | 58.01 | 7 |
| ATOM | 410 | CA | GLN | 96 | 94.798 | 270.177 | 117.798 | 1.00 | 62.01 | 6 |
| ATOM | 411 | CB | GLN | 96 | 95.359 | 269.456 | 119.037 | 1.00 | 71.46 | 6 |
| ATOM | 412 | CG | GLN | 96 | 96.483 | 270.193 | 119.815 | 1.00 | 79.76 | 6 |
| ATOM | 413 | CD | GLN | 96 | 95.982 | 271.072 | 120.957 | 1.00 | 86.55 | 6 |
| ATOM | 414 | OE1 | GLN | 96 | 95.459 | 272.170 | 120.739 | 1.00 | 88.23 | 8 |
| ATOM | 415 | NE2 | GLN | 96 | 96.170 | 270.600 | 122.187 | 1.00 | 86.54 | 7 |
| ATOM | 416 | C | GLN | 96 | 95.927 | 270.809 | 117.002 | 1.00 | 60.46 | 6 |
| ATOM | 417 | O | GLN | 96 | 96.461 | 271.839 | 117.400 | 1.00 | 70.38 | 8 |
| ATOM | 418 | N | ASP | 97 | 96.269 | 270.222 | 115.866 | 1.00 | 52.27 | 7 |
| ATOM | 419 | CA | ASP | 97 | 97.348 | 270.746 | 115.024 | 1.00 | 50.91 | 6 |
| ATOM | 420 | CB | ASP | 97 | 97.085 | 272.173 | 114.543 | 1.00 | 49.86 | 6 |
| ATOM | 421 | CG | ASP | 97 | 98.188 | 272.699 | 113.619 | 1.00 | 57.50 | 6 |
| ATOM | 422 | OD1 | ASP | 97 | 98.852 | 271.899 | 112.929 | 1.00 | 56.02 | 8 |
| ATOM | 423 | OD2 | ASP | 97 | 98.391 | 273.929 | 113.576 | 1.00 | 63.44 | 8 |
| ATOM | 424 | C | ASP | 97 | 98.709 | 270.687 | 115.687 | 1.00 | 50.91 | 6 |
| ATOM | 425 | O | ASP | 97 | 99.100 | 271.562 | 116.452 | 1.00 | 48.95 | 8 |
| ATOM | 426 | N | CYS | 98 | 99.408 | 269.609 | 115.385 | 1.00 | 53.53 | 7 |
| ATOM | 427 | CA | CYS | 98 | 100.737 | 269.340 | 115.892 | 1.00 | 53.91 | 6 |
| ATOM | 428 | CB | CYS | 98 | 100.677 | 268.209 | 116.903 | 1.00 | 54.20 | 6 |
| ATOM | 429 | SG | CYS | 98 | 99.754 | 266.827 | 116.282 | 1.00 | 65.92 | 16 |
| ATOM | 430 | C | CYS | 98 | 101.411 | 268.866 | 114.626 | 1.00 | 54.51 | 6 |
| ATOM | 431 | O | CYS | 98 | 100.775 | 268.830 | 113.570 | 1.00 | 56.51 | 8 |
| ATOM | 432 | N | PRO | 99 | 102.715 | 268.567 | 114.678 | 1.00 | 53.03 | 7 |
| ATOM | 433 | CD | PRO | 99 | 103.725 | 268.874 | 115.698 | 1.00 | 50.80 | 6 |
| ATOM | 434 | CA | PRO | 99 | 103.345 | 268.104 | 113.439 | 1.00 | 49.34 | 6 |
| ATOM | 435 | CB | PRO | 99 | 104.831 | 268.222 | 113.752 | 1.00 | 47.85 | 6 |
| ATOM | 436 | CG | PRO | 99 | 104.880 | 268.053 | 115.234 | 1.00 | 54.89 | 6 |
| ATOM | 437 | C | PRO | 99 | 102.892 | 266.698 | 112.996 | 1.00 | 46.94 | 6 |
| ATOM | 438 | O | PRO | 99 | 102.973 | 266.369 | 111.819 | 1.00 | 45.51 | 8 |
| ATOM | 439 | N | LYS | 100 | 102.362 | 265.903 | 113.924 | 1.00 | 46.00 | 7 |
| ATOM | 440 | CA | LYS | 100 | 101.891 | 264.556 | 113.600 | 1.00 | 49.79 | 6 |
| ATOM | 441 | CB | LYS | 100 | 101.720 | 263.722 | 114.871 | 1.00 | 55.32 | 6 |
| ATOM | 442 | CG | LYS | 100 | 101.310 | 262.272 | 114.621 | 1.00 | 65.19 | 6 |
| ATOM | 443 | CD | LYS | 100 | 101.091 | 261.476 | 115.923 | 1.00 | 78.43 | 6 |
| ATOM | 444 | CE | LYS | 100 | 99.723 | 261.755 | 116.576 | 1.00 | 86.39 | 6 |
| ATOM | 445 | NZ | LYS | 100 | 99.439 | 260.970 | 117.838 | 1.00 | 81.26 | 7 |
| ATOM | 446 | C | LYS | 100 | 100.553 | 264.625 | 112.849 | 1.00 | 53.43 | 6 |
| ATOM | 447 | O | LYS | 100 | 100.232 | 263.739 | 112.064 | 1.00 | 53.34 | 8 |
| ATOM | 448 | N | ALA | 101 | 99.752 | 265.655 | 113.130 | 1.00 | 57.24 | 7 |
| ATOM | 449 | CA | ALA | 101 | 98.452 | 265.838 | 112.476 | 1.00 | 53.76 | 6 |
| ATOM | 450 | CB | ALA | 101 | 97.602 | 266.806 | 113.251 | 1.00 | 47.38 | 6 |
| ATOM | 451 | C | ALA | 101 | 98.703 | 266.369 | 111.067 | 1.00 | 55.06 | 6 |
| ATOM | 452 | O | ALA | 101 | 98.135 | 265.870 | 110.095 | 1.00 | 55.05 | 8 |
| ATOM | 453 | N | ARG | 102 | 99.578 | 267.367 | 110.964 | 1.00 | 53.35 | 7 |
| ATOM | 454 | CA | ARG | 102 | 99.919 | 267.963 | 109.683 | 1.00 | 57.33 | 6 |
| ATOM | 455 | CB | ARG | 102 | 101.032 | 268.997 | 109.864 | 1.00 | 57.65 | 6 |
| ATOM | 456 | CG | ARG | 102 | 100.601 | 270.370 | 110.374 | 1.00 | 67.20 | 6 |
| ATOM | 457 | CD | ARG | 102 | 101.739 | 271.098 | 111.118 | 1.00 | 76.53 | 6 |
| ATOM | 458 | NE | ARG | 102 | 102.992 | 271.117 | 110.354 | 1.00 | 90.17 | 7 |
| ATOM | 459 | CZ | ARG | 102 | 104.207 | 270.929 | 110.873 | 1.00 | 92.00 | 6 |
| ATOM | 460 | NH1 | ARG | 102 | 104.360 | 270.714 | 112.175 | 1.00 | 88.60 | 7 |
| ATOM | 461 | NH2 | ARG | 102 | 105.271 | 270.896 | 110.074 | 1.00 | 90.41 | 7 |
| ATOM | 462 | C | ARG | 102 | 100.411 | 266.850 | 108.758 | 1.00 | 61.79 | 6 |
| ATOM | 463 | O | ARG | 102 | 100.158 | 266.865 | 107.555 | 1.00 | 66.20 | 8 |
| ATOM | 464 | N | ARG | 103 | 101.057 | 265.847 | 109.345 | 1.00 | 62.64 | 7 |
| ATOM | 465 | CA | ARG | 103 | 101.589 | 264.719 | 108.588 | 1.00 | 60.89 | 6 |
| ATOM | 466 | CB | ARG | 103 | 102.681 | 264.033 | 109.410 | 1.00 | 60.83 | 6 |
| ATOM | 467 | CG | ARG | 103 | 103.504 | 263.002 | 108.657 | 1.00 | 66.42 | 6 |
| ATOM | 468 | CD | ARG | 103 | 104.803 | 262.680 | 109.395 | 1.00 | 69.20 | 6 |
| ATOM | 469 | NE | ARG | 103 | 104.572 | 262.408 | 110.812 | 1.00 | 69.35 | 7 |
| ATOM | 470 | CZ | ARG | 103 | 105.142 | 263.076 | 111.809 | 1.00 | 70.13 | 6 |
| ATOM | 471 | NH1 | ARG | 103 | 105.993 | 264.064 | 111.568 | 1.00 | 71.72 | 7 |
| ATOM | 472 | NH2 | ARG | 103 | 104.830 | 262.779 | 113.056 | 1.00 | 73.81 | 7 |
| ATOM | 473 | C | ARG | 103 | 100.531 | 263.710 | 108.130 | 1.00 | 60.06 | 6 |
| ATOM | 474 | O | ARG | 103 | 100.595 | 263.197 | 107.008 | 1.00 | 60.04 | 8 |
| ATOM | 475 | N | GLU | 104 | 99.527 | 263.475 | 108.967 | 1.00 | 58.04 | 7 |
| ATOM | 476 | CA | GLU | 104 | 98.475 | 262.528 | 108.620 | 1.00 | 56.01 | 6 |
| ATOM | 477 | CB | GLU | 104 | 97.767 | 262.037 | 109.876 | 1.00 | 58.13 | 6 |
| ATOM | 478 | CG | GLU | 104 | 98.753 | 261.438 | 110.856 | 1.00 | 69.69 | 6 |
| ATOM | 479 | CD | GLU | 104 | 98.104 | 260.709 | 112.005 | 1.00 | 76.95 | 6 |
| ATOM | 480 | OE1 | GLU | 104 | 98.323 | 259.485 | 112.113 | 1.00 | 82.32 | 8 |
| ATOM | 481 | OE2 | GLU | 104 | 97.405 | 261.354 | 112.813 | 1.00 | 81.38 | 8 |
| ATOM | 482 | C | GLU | 104 | 97.499 | 263.026 | 107.557 | 1.00 | 53.22 | 6 |
| ATOM | 483 | O | GLU | 104 | 97.165 | 262.276 | 106.641 | 1.00 | 50.63 | 8 |
| ATOM | 484 | N | VAL | 105 | 97.072 | 264.290 | 107.646 | 1.00 | 51.15 | 7 |
| ATOM | 485 | CA | VAL | 105 | 96.140 | 264.837 | 106.652 | 1.00 | 48.46 | 6 |
| ATOM | 486 | CB | VAL | 105 | 95.653 | 266.280 | 106.992 | 1.00 | 44.02 | 6 |
| ATOM | 487 | CG1 | VAL | 105 | 95.000 | 266.310 | 108.339 | 1.00 | 51.39 | 6 |
| ATOM | 488 | CG2 | VAL | 105 | 96.794 | 267.267 | 106.952 | 1.00 | 42.57 | 6 |
| ATOM | 489 | C | VAL | 105 | 96.852 | 264.896 | 105.304 | 1.00 | 49.32 | 6 |
| ATOM | 490 | O | VAL | 105 | 96.255 | 264.684 | 104.246 | 1.00 | 48.10 | 8 |
| ATOM | 491 | N | GLU | 106 | 98.152 | 265.153 | 105.364 | 1.00 | 48.27 | 7 |
| ATOM | 492 | CA | GLU | 106 | 98.959 | 265.247 | 104.172 | 1.00 | 45.34 | 6 |
| ATOM | 493 | CB | GLU | 106 | 100.345 | 265.751 | 104.559 | 1.00 | 47.84 | 6 |
| ATOM | 494 | CG | GLU | 106 | 100.973 | 266.701 | 103.552 | 1.00 | 61.82 | 6 |
| ATOM | 495 | CD | GLU | 106 | 101.917 | 265.972 | 102.601 | 1.00 | 78.09 | 6 |
| ATOM | 496 | OE1 | GLU | 106 | 101.419 | 265.334 | 101.636 | 1.00 | 80.27 | 8 |
| ATOM | 497 | OE2 | GLU | 106 | 103.154 | 266.020 | 102.835 | 1.00 | 78.34 | 8 |
| ATOM | 498 | C | GLU | 106 | 98.991 | 263.895 | 103.475 | 1.00 | 45.77 | 6 |
| ATOM | 499 | O | GLU | 106 | 99.230 | 263.814 | 102.273 | 1.00 | 46.16 | 8 |
| ATOM | 500 | N | LEU | 107 | 98.715 | 262.832 | 104.228 | 1.00 | 44.59 | 7 |
| ATOM | 501 | CA | LEU | 107 | 98.709 | 261.485 | 103.667 | 1.00 | 42.00 | 6 |
| ATOM | 502 | CB | LEU | 107 | 98.979 | 260.433 | 104.740 | 1.00 | 44.06 | 6 |
| ATOM | 503 | CG | LEU | 107 | 100.439 | 260.131 | 105.103 | 1.00 | 39.64 | 6 |
| ATOM | 504 | CD1 | LEU | 107 | 100.485 | 259.244 | 106.312 | 1.00 | 36.64 | 6 |
| ATOM | 505 | CD2 | LEU | 107 | 101.114 | 259.462 | 103.938 | 1.00 | 37.18 | 6 |
| ATOM | 506 | C | LEU | 107 | 97.368 | 261.260 | 103.024 | 1.00 | 43.35 | 6 |
| ATOM | 507 | O | LEU | 107 | 97.299 | 260.738 | 101.930 | 1.00 | 46.96 | 8 |
| ATOM | 508 | N | HIS | 108 | 96.307 | 261.686 | 103.710 | 1.00 | 45.35 | 7 |
| ATOM | 509 | CA | HIS | 108 | 94.920 | 261.562 | 103.234 | 1.00 | 44.94 | 6 |
| ATOM | 510 | CB | HIS | 108 | 93.995 | 262.190 | 104.270 | 1.00 | 40.43 | 6 |
| ATOM | 511 | CG | HIS | 108 | 92.545 | 261.909 | 104.054 | 1.00 | 39.88 | 6 |
| ATOM | 512 | CD2 | HIS | 108 | 91.885 | 261.345 | 103.014 | 1.00 | 38.35 | 6 |
| ATOM | 513 | ND1 | HIS | 108 | 91.589 | 262.212 | 105.000 | 1.00 | 40.72 | 7 |
| ATOM | 514 | CE1 | HIS | 108 | 90.400 | 261.843 | 104.553 | 1.00 | 41.23 | 6 |
| ATOM | 515 | NE2 | HIS | 108 | 90.553 | 261.313 | 103.352 | 1.00 | 35.89 | 7 |
| ATOM | 516 | C | HIS | 108 | 94.801 | 262.302 | 101.883 | 1.00 | 49.84 | 6 |
| ATOM | 517 | O | HIS | 108 | 94.172 | 261.813 | 100.953 | 1.00 | 51.83 | 8 |
| ATOM | 518 | N | TRP | 109 | 95.398 | 263.491 | 101.805 | 1.00 | 51.27 | 7 |
| ATOM | 519 | CA | TRP | 109 | 95.400 | 264.317 | 100.597 | 1.00 | 49.60 | 6 |
| ATOM | 520 | CB | TRP | 109 | 95.727 | 265.774 | 100.963 | 1.00 | 49.30 | 6 |
| ATOM | 521 | CG | TRP | 109 | 95.997 | 266.662 | 99.760 | 1.00 | 48.30 | 6 |
| ATOM | 522 | CD2 | TRP | 109 | 95.051 | 267.506 | 99.112 | 1.00 | 50.26 | 6 |
| ATOM | 523 | CE2 | TRP | 109 | 95.693 | 268.072 | 97.993 | 1.00 | 55.71 | 6 |
| ATOM | 524 | CE3 | TRP | 109 | 93.711 | 267.836 | 99.364 | 1.00 | 51.64 | 6 |
| ATOM | 525 | CD1 | TRP | 109 | 97.164 | 266.762 | 99.034 | 1.00 | 46.93 | 6 |
| ATOM | 526 | NE1 | TRP | 109 | 96.982 | 267.600 | 97.969 | 1.00 | 52.62 | 7 |
| ATOM | 527 | CZ2 | TRP | 109 | 95.032 | 268.955 | 97.124 | 1.00 | 62.74 | 6 |
| ATOM | 528 | CZ3 | TRP | 109 | 93.056 | 268.711 | 98.500 | 1.00 | 51.35 | 6 |
| ATOM | 529 | CH2 | TRP | 109 | 93.715 | 269.258 | 97.397 | 1.00 | 58.44 | 6 |
| ATOM | 530 | C | TRP | 109 | 96.595 | 263.747 | 99.860 | 1.00 | 52.52 | 6 |
| ATOM | 531 | O | TRP | 109 | 97.712 | 264.189 | 100.070 | 1.00 | 67.99 | 8 |
| ATOM | 532 | N | ARG | 110 | 96.391 | 262.733 | 99.049 | 1.00 | 45.97 | 7 |
| ATOM | 533 | CA | ARG | 110 | 97.492 | 262.107 | 98.301 | 1.00 | 41.85 | 6 |
| ATOM | 534 | CB | ARG | 110 | 98.669 | 261.778 | 99.220 | 1.00 | 35.57 | 6 |
| ATOM | 535 | CG | ARG | 110 | 100.009 | 262.248 | 98.697 | 1.00 | 44.63 | 6 |
| ATOM | 536 | CD | ARG | 110 | 101.092 | 262.412 | 99.797 | 1.00 | 51.06 | 6 |
| ATOM | 537 | NE | ARG | 110 | 101.614 | 261.134 | 100.285 | 1.00 | 54.76 | 7 |
| ATOM | 538 | CZ | ARG | 110 | 102.437 | 260.341 | 99.604 | 1.00 | 54.90 | 6 |
| ATOM | 539 | NH1 | ARG | 110 | 102.859 | 260.687 | 98.401 | 1.00 | 56.96 | 7 |
| ATOM | 540 | NH2 | ARG | 110 | 102.787 | 259.165 | 100.101 | 1.00 | 54.78 | 7 |
| ATOM | 541 | C | ARG | 110 | 96.796 | 260.840 | 97.903 | 1.00 | 45.91 | 6 |
| ATOM | 542 | O | ARG | 110 | 96.825 | 260.422 | 96.746 | 1.00 | 53.36 | 8 |
| ATOM | 543 | N | ALA | 111 | 96.072 | 260.316 | 98.886 | 1.00 | 40.86 | 7 |
| ATOM | 544 | CA | ALA | 111 | 95.297 | 259.115 | 98.766 | 1.00 | 41.11 | 6 |
| ATOM | 545 | CB | ALA | 111 | 95.167 | 258.469 | 100.125 | 1.00 | 43.86 | 6 |
| ATOM | 546 | C | ALA | 111 | 93.931 | 259.552 | 98.283 | 1.00 | 43.14 | 6 |
| ATOM | 547 | O | ALA | 111 | 93.100 | 258.713 | 97.978 | 1.00 | 49.96 | 8 |
| ATOM | 548 | N | SER | 112 | 93.715 | 260.868 | 98.210 | 1.00 | 49.04 | 7 |
| ATOM | 549 | CA | SER | 112 | 92.444 | 261.467 | 97.774 | 1.00 | 52.26 | 6 |
| ATOM | 550 | CB | SER | 112 | 92.385 | 262.935 | 98.169 | 1.00 | 55.06 | 6 |
| ATOM | 551 | OG | SER | 112 | 92.121 | 263.079 | 99.555 | 1.00 | 65.98 | 8 |
| ATOM | 552 | C | SER | 112 | 92.006 | 261.329 | 96.329 | 1.00 | 52.46 | 6 |
| ATOM | 553 | O | SER | 112 | 90.837 | 261.541 | 96.032 | 1.00 | 49.95 | 8 |
| ATOM | 554 | N | GLN | 113 | 92.939 | 261.016 | 95.432 | 1.00 | 56.12 | 7 |
| ATOM | 555 | CA | GLN | 113 | 92.616 | 260.853 | 94.018 | 1.00 | 59.35 | 6 |
| ATOM | 556 | CB | GLN | 113 | 93.806 | 261.230 | 93.115 | 1.00 | 66.37 | 6 |
| ATOM | 557 | CG | GLN | 113 | 94.518 | 262.528 | 93.483 | 1.00 | 78.46 | 6 |
| ATOM | 558 | CD | GLN | 113 | 93.565 | 263.688 | 93.749 | 1.00 | 85.18 | 6 |
| ATOM | 559 | OE1 | GLN | 113 | 93.538 | 264.244 | 94.855 | 1.00 | 84.58 | 8 |
| ATOM | 560 | NE2 | GLN | 113 | 92.779 | 264.061 | 92.737 | 1.00 | 87.90 | 7 |
| ATOM | 561 | C | GLN | 113 | 92.256 | 259.390 | 93.808 | 1.00 | 58.14 | 6 |
| ATOM | 562 | O | GLN | 113 | 92.648 | 258.764 | 92.828 | 1.00 | 63.76 | 8 |
| ATOM | 563 | N | CYS | 114 | 91.548 | 258.826 | 94.767 | 1.00 | 56.79 | 7 |
| ATOM | 564 | CA | CYS | 114 | 91.147 | 257.445 | 94.677 | 1.00 | 59.72 | 6 |
| ATOM | 565 | CB | CYS | 114 | 91.916 | 256.602 | 95.676 | 1.00 | 60.66 | 6 |
| ATOM | 566 | SG | CYS | 114 | 91.109 | 255.057 | 95.975 | 1.00 | 71.13 | 16 |
| ATOM | 567 | C | CYS | 114 | 89.672 | 257.404 | 94.996 | 1.00 | 62.75 | 6 |
| ATOM | 568 | O | CYS | 114 | 89.265 | 257.704 | 96.122 | 1.00 | 66.87 | 8 |
| ATOM | 569 | N | PRO | 115 | 88.849 | 257.010 | 94.013 | 1.00 | 62.36 | 7 |
| ATOM | 570 | CD | PRO | 115 | 89.294 | 256.445 | 92.733 | 1.00 | 60.83 | 6 |
| ATOM | 571 | CA | PRO | 115 | 87.390 | 256.914 | 94.130 | 1.00 | 60.74 | 6 |
| ATOM | 572 | CB | PRO | 115 | 86.992 | 256.124 | 92.880 | 1.00 | 58.69 | 6 |
| ATOM | 573 | CG | PRO | 115 | 88.255 | 255.392 | 92.512 | 1.00 | 65.07 | 6 |
| ATOM | 574 | C | PRO | 115 | 86.809 | 256.305 | 95.408 | 1.00 | 59.83 | 6 |
| ATOM | 575 | O | PRO | 115 | 85.714 | 256.692 | 95.820 | 1.00 | 62.08 | 8 |
| ATOM | 576 | N | HIS | 116 | 87.527 | 255.388 | 96.056 | 1.00 | 56.30 | 7 |
| ATOM | 577 | CA | HIS | 116 | 86.990 | 254.793 | 97.272 | 1.00 | 52.90 | 6 |
| ATOM | 578 | CB | HIS | 116 | 87.027 | 253.274 | 97.210 | 1.00 | 51.70 | 6 |
| ATOM | 579 | CG | HIS | 116 | 85.999 | 252.690 | 96.298 | 1.00 | 52.79 | 6 |
| ATOM | 580 | CD2 | HIS | 116 | 84.745 | 252.245 | 96.536 | 1.00 | 52.48 | 6 |
| ATOM | 581 | ND1 | HIS | 116 | 86.213 | 252.520 | 94.946 | 1.00 | 56.60 | 7 |
| ATOM | 582 | CE1 | HIS | 116 | 85.135 | 251.996 | 94.392 | 1.00 | 52.78 | 6 |
| ATOM | 583 | NE2 | HIS | 116 | 84.231 | 251.817 | 95.336 | 1.00 | 50.42 | 7 |
| ATOM | 584 | C | HIS | 116 | 87.517 | 255.283 | 98.599 | 1.00 | 53.01 | 6 |
| ATOM | 585 | O | HIS | 116 | 87.283 | 254.648 | 99.622 | 1.00 | 58.06 | 8 |
| ATOM | 586 | N | ILE | 117 | 88.204 | 256.417 | 98.599 | 1.00 | 47.98 | 7 |
| ATOM | 587 | CA | ILE | 117 | 88.742 | 256.975 | 99.832 | 1.00 | 43.43 | 6 |
| ATOM | 588 | CB | ILE | 117 | 90.269 | 257.013 | 99.811 | 1.00 | 42.89 | 6 |
| ATOM | 589 | CG2 | ILE | 117 | 90.801 | 257.841 | 100.963 | 1.00 | 44.01 | 6 |
| ATOM | 590 | CG1 | ILE | 117 | 90.820 | 255.596 | 99.926 | 1.00 | 44.44 | 6 |
| ATOM | 591 | CD1 | ILE | 117 | 92.303 | 255.531 | 99.826 | 1.00 | 45.46 | 6 |
| ATOM | 592 | C | ILE | 117 | 88.164 | 258.378 | 99.900 | 1.00 | 44.72 | 6 |
| ATOM | 593 | O | ILE | 117 | 88.309 | 259.134 | 98.947 | 1.00 | 49.50 | 8 |
| ATOM | 594 | N | VAL | 118 | 87.465 | 258.690 | 100.999 | 1.00 | 43.07 | 7 |
| ATOM | 595 | CA | VAL | 118 | 86.835 | 259.996 | 101.221 | 1.00 | 38.87 | 6 |
| ATOM | 596 | CB | VAL | 118 | 86.306 | 260.121 | 102.655 | 1.00 | 40.23 | 6 |
| ATOM | 597 | CG1 | VAL | 118 | 87.414 | 260.478 | 103.635 | 1.00 | 42.59 | 6 |
| ATOM | 598 | CG2 | VAL | 118 | 85.206 | 261.133 | 102.691 | 1.00 | 46.81 | 6 |
| ATOM | 599 | C | VAL | 118 | 87.764 | 261.153 | 100.868 | 1.00 | 38.89 | 6 |
| ATOM | 600 | O | VAL | 118 | 88.879 | 261.243 | 101.379 | 1.00 | 39.66 | 8 |
| ATOM | 601 | N | ARG | 119 | 87.284 | 262.050 | 100.011 | 1.00 | 38.95 | 7 |
| ATOM | 602 | CA | ARG | 119 | 88.062 | 263.195 | 99.558 | 1.00 | 37.81 | 6 |
| ATOM | 603 | CB | ARG | 119 | 87.553 | 263.634 | 98.197 | 1.00 | 37.92 | 6 |
| ATOM | 604 | CG | ARG | 119 | 87.959 | 265.039 | 97.819 | 1.00 | 53.56 | 6 |
| ATOM | 605 | CD | ARG | 119 | 87.549 | 265.412 | 96.402 | 1.00 | 65.48 | 6 |
| ATOM | 606 | NE | ARG | 119 | 88.604 | 265.153 | 95.425 | 1.00 | 66.50 | 7 |
| ATOM | 607 | CZ | ARG | 119 | 88.571 | 264.158 | 94.548 | 1.00 | 68.27 | 6 |
| ATOM | 608 | NH1 | ARG | 119 | 87.533 | 263.327 | 94.528 | 1.00 | 71.30 | 7 |
| ATOM | 609 | NH2 | ARG | 119 | 89.567 | 264.002 | 93.682 | 1.00 | 66.97 | 7 |
| ATOM | 610 | C | ARG | 119 | 88.240 | 264.425 | 100.414 | 1.00 | 38.22 | 6 |
| ATOM | 611 | O | ARG | 119 | 87.255 | 265.030 | 100.820 | 1.00 | 41.60 | 8 |
| ATOM | 612 | N | ILE | 120 | 89.502 | 264.792 | 100.677 | 1.00 | 37.19 | 7 |
| ATOM | 613 | CA | ILE | 120 | 89.797 | 265.980 | 101.477 | 1.00 | 33.79 | 6 |
| ATOM | 614 | CB | ILE | 120 | 91.218 | 266.009 | 102.067 | 1.00 | 35.24 | 6 |
| ATOM | 615 | CG2 | ILE | 120 | 91.706 | 267.466 | 102.234 | 1.00 | 29.10 | 6 |
| ATOM | 616 | CG1 | ILE | 120 | 91.213 | 265.274 | 103.410 | 1.00 | 37.06 | 6 |
| ATOM | 617 | CD1 | ILE | 120 | 92.479 | 265.428 | 104.262 | 1.00 | 35.77 | 6 |
| ATOM | 618 | C | ILE | 120 | 89.667 | 267.057 | 100.435 | 1.00 | 38.15 | 6 |
| ATOM | 619 | O | ILE | 120 | 90.312 | 267.009 | 99.381 | 1.00 | 42.06 | 8 |
| ATOM | 620 | N | VAL | 121 | 88.796 | 268.006 | 100.727 | 1.00 | 39.84 | 7 |
| ATOM | 621 | CA | VAL | 121 | 88.521 | 269.120 | 99.835 | 1.00 | 36.51 | 6 |
| ATOM | 622 | CB | VAL | 121 | 87.007 | 269.164 | 99.597 | 1.00 | 29.88 | 6 |
| ATOM | 623 | CG1 | VAL | 121 | 86.464 | 270.529 | 99.714 | 1.00 | 33.08 | 6 |
| ATOM | 624 | CG2 | VAL | 121 | 86.699 | 268.542 | 98.281 | 1.00 | 29.00 | 6 |
| ATOM | 625 | C | VAL | 121 | 89.091 | 270.485 | 100.239 | 1.00 | 39.45 | 6 |
| ATOM | 626 | O | VAL | 121 | 89.252 | 271.359 | 99.394 | 1.00 | 40.76 | 8 |
| ATOM | 627 | N | ASP | 122 | 89.459 | 270.640 | 101.507 | 1.00 | 41.18 | 7 |
| ATOM | 628 | CA | ASP | 122 | 90.010 | 271.900 | 101.995 | 1.00 | 40.56 | 6 |
| ATOM | 629 | CB | ASP | 122 | 88.876 | 272.923 | 102.138 | 1.00 | 47.35 | 6 |
| ATOM | 630 | CG | ASP | 122 | 88.872 | 273.973 | 101.045 | 1.00 | 46.11 | 6 |
| ATOM | 631 | OD1 | ASP | 122 | 89.847 | 274.058 | 100.264 | 1.00 | 47.72 | 8 |
| ATOM | 632 | OD2 | ASP | 122 | 87.878 | 274.721 | 100.987 | 1.00 | 45.35 | 8 |
| ATOM | 633 | C | ASP | 122 | 90.632 | 271.711 | 103.366 | 1.00 | 40.26 | 6 |
| ATOM | 634 | O | ASP | 122 | 90.055 | 271.029 | 104.205 | 1.00 | 46.40 | 8 |
| ATOM | 635 | N | VAL | 123 | 91.787 | 272.324 | 103.610 | 1.00 | 36.94 | 7 |
| ATOM | 636 | CA | VAL | 123 | 92.441 | 272.196 | 104.913 | 1.00 | 34.79 | 6 |
| ATOM | 637 | CB | VAL | 123 | 93.750 | 271.349 | 104.886 | 1.00 | 34.97 | 6 |
| ATOM | 638 | CG1 | VAL | 123 | 94.197 | 271.065 | 106.287 | 1.00 | 36.05 | 6 |
| ATOM | 639 | CG2 | VAL | 123 | 93.585 | 270.052 | 104.109 | 1.00 | 33.40 | 6 |
| ATOM | 640 | C | VAL | 123 | 92.811 | 273.588 | 105.418 | 1.00 | 37.15 | 6 |
| ATOM | 641 | O | VAL | 123 | 93.600 | 274.309 | 104.800 | 1.00 | 40.68 | 8 |
| ATOM | 642 | N | TYR | 124 | 92.247 | 273.955 | 106.558 | 1.00 | 39.65 | 7 |
| ATOM | 643 | CA | TYR | 124 | 92.508 | 275.251 | 107.164 | 1.00 | 40.57 | 6 |
| ATOM | 644 | CB | TYR | 124 | 91.192 | 275.944 | 107.534 | 1.00 | 39.39 | 6 |
| ATOM | 645 | CG | TYR | 124 | 90.394 | 276.371 | 106.341 | 1.00 | 36.94 | 6 |
| ATOM | 646 | CD1 | TYR | 124 | 90.547 | 277.654 | 105.814 | 1.00 | 39.91 | 6 |
| ATOM | 647 | CE1 | TYR | 124 | 89.921 | 278.032 | 104.648 | 1.00 | 37.05 | 6 |
| ATOM | 648 | CD2 | TYR | 124 | 89.574 | 275.475 | 105.672 | 1.00 | 36.08 | 6 |
| ATOM | 649 | CE2 | TYR | 124 | 88.944 | 275.844 | 104.500 | 1.00 | 46.34 | 6 |
| ATOM | 650 | CZ | TYR | 124 | 89.126 | 277.126 | 103.991 | 1.00 | 42.79 | 6 |
| ATOM | 651 | OH | TYR | 124 | 88.539 | 277.482 | 102.803 | 1.00 | 45.73 | 8 |
| ATOM | 652 | C | TYR | 124 | 93.339 | 275.130 | 108.429 | 1.00 | 45.50 | 6 |
| ATOM | 653 | O | TYR | 124 | 93.305 | 274.110 | 109.135 | 1.00 | 46.67 | 8 |
| ATOM | 654 | N | GLU | 125 | 94.038 | 276.218 | 108.727 | 1.00 | 45.98 | 7 |
| ATOM | 655 | CA | GLU | 125 | 94.880 | 276.315 | 109.896 | 1.00 | 45.17 | 6 |
| ATOM | 656 | CB | GLU | 125 | 96.346 | 276.312 | 109.513 | 1.00 | 49.65 | 6 |
| ATOM | 657 | CG | GLU | 125 | 97.225 | 276.399 | 110.742 | 1.00 | 74.22 | 6 |
| ATOM | 658 | CD | GLU | 125 | 98.649 | 275.961 | 110.497 | 1.00 | 86.81 | 6 |
| ATOM | 659 | OE1 | GLU | 125 | 98.876 | 274.762 | 110.157 | 1.00 | 87.23 | 8 |
| ATOM | 660 | OE2 | GLU | 125 | 99.539 | 276.829 | 110.671 | 1.00 | 95.16 | 8 |
| ATOM | 661 | C | GLU | 125 | 94.510 | 277.631 | 110.571 | 1.00 | 45.43 | 6 |
| ATOM | 662 | O | GLU | 125 | 95.118 | 278.666 | 110.302 | 1.00 | 47.22 | 8 |
| ATOM | 663 | N | ASN | 126 | 93.494 | 277.576 | 111.430 | 1.00 | 43.90 | 7 |
| ATOM | 664 | CA | ASN | 126 | 92.997 | 278.742 | 112.162 | 1.00 | 46.65 | 6 |
| ATOM | 665 | CB | ASN | 126 | 91.514 | 278.966 | 111.838 | 1.00 | 43.85 | 6 |
| ATOM | 666 | CG | ASN | 126 | 91.254 | 279.129 | 110.359 | 1.00 | 46.86 | 6 |
| ATOM | 667 | OD1 | ASN | 126 | 90.141 | 278.940 | 109.905 | 1.00 | 48.51 | 8 |
| ATOM | 668 | ND2 | ASN | 126 | 92.276 | 279.498 | 109.606 | 1.00 | 46.98 | 7 |
| ATOM | 669 | C | ASN | 126 | 93.134 | 278.675 | 113.674 | 1.00 | 46.10 | 6 |
| ATOM | 670 | O | ASN | 126 | 93.118 | 277.604 | 114.254 | 1.00 | 51.28 | 8 |
| ATOM | 671 | N | LEU | 127 | 93.226 | 279.843 | 114.298 | 1.00 | 46.48 | 7 |
| ATOM | 672 | CA | LEU | 127 | 93.352 | 279.981 | 115.745 | 1.00 | 46.37 | 6 |
| ATOM | 673 | CB | LEU | 127 | 93.945 | 281.334 | 116.087 | 1.00 | 45.87 | 6 |
| ATOM | 674 | CG | LEU | 127 | 95.417 | 281.436 | 116.400 | 1.00 | 49.01 | 6 |
| ATOM | 675 | CD1 | LEU | 127 | 95.665 | 282.803 | 116.954 | 1.00 | 51.50 | 6 |
| ATOM | 676 | CD2 | LEU | 127 | 95.784 | 280.385 | 117.418 | 1.00 | 53.61 | 6 |
| ATOM | 677 | C | LEU | 127 | 92.002 | 279.938 | 116.437 | 1.00 | 50.25 | 6 |
| ATOM | 678 | O | LEU | 127 | 91.115 | 280.707 | 116.098 | 1.00 | 51.90 | 8 |
| ATOM | 679 | N | TYR | 128 | 91.849 | 279.084 | 117.438 | 1.00 | 57.66 | 7 |
| ATOM | 680 | CA | TYR | 128 | 90.581 | 279.017 | 118.135 | 1.00 | 67.26 | 6 |
| ATOM | 681 | CB | TYR | 128 | 90.171 | 277.571 | 118.409 | 1.00 | 72.99 | 6 |
| ATOM | 682 | CG | TYR | 128 | 88.675 | 277.376 | 118.601 | 1.00 | 80.33 | 6 |
| ATOM | 683 | CD1 | TYR | 128 | 88.060 | 276.184 | 118.213 | 1.00 | 85.27 | 6 |
| ATOM | 684 | CE1 | TYR | 128 | 86.687 | 275.983 | 118.402 | 1.00 | 91.31 | 6 |
| ATOM | 685 | CD2 | TYR | 128 | 87.877 | 278.373 | 119.185 | 1.00 | 80.56 | 6 |
| ATOM | 686 | CE2 | TYR | 128 | 86.507 | 278.191 | 119.382 | 1.00 | 84.39 | 6 |
| ATOM | 687 | CZ | TYR | 128 | 85.912 | 276.988 | 118.989 | 1.00 | 92.94 | 6 |
| ATOM | 688 | OH | TYR | 128 | 84.554 | 276.773 | 119.183 | 1.00 | 94.57 | 8 |
| ATOM | 689 | C | TYR | 128 | 90.728 | 279.841 | 119.429 | 1.00 | 72.05 | 6 |
| ATOM | 690 | O | TYR | 128 | 90.868 | 281.074 | 119.367 | 1.00 | 75.92 | 8 |
| ATOM | 691 | N | ALA | 129 | 90.718 | 279.195 | 120.592 | 1.00 | 69.32 | 7 |
| ATOM | 692 | CA | ALA | 129 | 90.854 | 279.946 | 121.834 | 1.00 | 69.96 | 6 |
| ATOM | 693 | CB | ALA | 129 | 90.210 | 279.185 | 122.974 | 1.00 | 71.00 | 6 |
| ATOM | 694 | C | ALA | 129 | 92.336 | 280.143 | 122.101 | 1.00 | 72.34 | 6 |
| ATOM | 695 | O | ALA | 129 | 92.862 | 279.634 | 123.088 | 1.00 | 77.83 | 8 |
| ATOM | 696 | N | GLY | 130 | 93.017 | 280.877 | 121.223 | 1.00 | 70.46 | 7 |
| ATOM | 697 | CA | GLY | 130 | 94.449 | 281.068 | 121.382 | 1.00 | 65.45 | 6 |
| ATOM | 698 | C | GLY | 130 | 95.251 | 279.840 | 120.953 | 1.00 | 62.00 | 6 |
| ATOM | 699 | O | GLY | 130 | 96.447 | 279.941 | 120.711 | 1.00 | 59.82 | 8 |
| ATOM | 700 | N | ARG | 131 | 94.596 | 278.681 | 120.884 | 1.00 | 61.34 | 7 |
| ATOM | 701 | CA | ARG | 131 | 95.224 | 277.426 | 120.470 | 1.00 | 62.10 | 6 |
| ATOM | 702 | CB | ARG | 131 | 94.569 | 276.234 | 121.188 | 1.00 | 72.05 | 6 |
| ATOM | 703 | CG | ARG | 131 | 94.807 | 276.052 | 122.698 | 1.00 | 83.50 | 6 |
| ATOM | 704 | CD | ARG | 131 | 94.232 | 274.670 | 123.112 | 1.00 | 93.50 | 6 |
| ATOM | 705 | NE | ARG | 131 | 94.244 | 274.361 | 124.550 | 1.00 | 98.15 | 7 |
| ATOM | 706 | CZ | ARG | 131 | 93.539 | 273.369 | 125.107 | 1.00 | 99.21 | 6 |
| ATOM | 707 | NH1 | ARG | 131 | 92.762 | 272.589 | 124.363 | 1.00 | 94.93 | 7 |
| ATOM | 708 | NH2 | ARG | 131 | 93.620 | 273.135 | 126.411 | 1.00 | 98.95 | 7 |
| ATOM | 709 | C | ARG | 131 | 95.046 | 277.200 | 118.958 | 1.00 | 57.49 | 6 |
| ATOM | 710 | O | ARG | 131 | 93.940 | 277.307 | 118.450 | 1.00 | 57.51 | 8 |
| ATOM | 711 | N | LYS | 132 | 96.110 | 276.811 | 118.264 | 1.00 | 51.73 | 7 |
| ATOM | 712 | CA | LYS | 132 | 96.050 | 276.562 | 116.822 | 1.00 | 48.76 | 6 |
| ATOM | 713 | CB | LYS | 132 | 97.452 | 276.483 | 116.235 | 1.00 | 45.36 | 6 |
| ATOM | 714 | CG | LYS | 132 | 98.309 | 277.684 | 116.375 | 1.00 | 52.39 | 6 |
| ATOM | 715 | CD | LYS | 132 | 99.696 | 277.343 | 115.847 | 1.00 | 55.25 | 6 |
| ATOM | 716 | CE | LYS | 132 | 99.660 | 276.845 | 114.401 | 1.00 | 57.49 | 6 |
| ATOM | 717 | NZ | LYS | 132 | 101.037 | 276.596 | 113.850 | 1.00 | 56.75 | 7 |
| ATOM | 718 | C | LYS | 132 | 95.359 | 275.261 | 116.362 | 1.00 | 52.12 | 6 |
| ATOM | 719 | O | LYS | 132 | 96.011 | 274.225 | 116.266 | 1.00 | 55.37 | 8 |
| ATOM | 720 | N | CYS | 133 | 94.075 | 275.302 | 116.031 | 1.00 | 52.86 | 7 |
| ATOM | 721 | CA | CYS | 133 | 93.398 | 274.093 | 115.571 | 1.00 | 55.23 | 6 |
| ATOM | 722 | CB | CYS | 133 | 91.911 | 274.144 | 115.921 | 1.00 | 66.98 | 6 |
| ATOM | 723 | SG | CYS | 133 | 91.479 | 273.424 | 117.557 | 1.00 | 93.79 | 16 |
| ATOM | 724 | C | CYS | 133 | 93.580 | 273.923 | 114.045 | 1.00 | 50.94 | 6 |
| ATOM | 725 | O | CYS | 133 | 93.731 | 274.899 | 113.318 | 1.00 | 45.59 | 8 |
| ATOM | 726 | N | LEU | 134 | 93.613 | 272.676 | 113.577 | 1.00 | 48.19 | 7 |
| ATOM | 727 | CA | LEU | 134 | 93.779 | 272.352 | 112.149 | 1.00 | 43.79 | 6 |
| ATOM | 728 | CB | LEU | 134 | 94.890 | 271.318 | 111.982 | 1.00 | 36.69 | 6 |
| ATOM | 729 | CG | LEU | 134 | 95.073 | 270.665 | 110.625 | 1.00 | 34.12 | 6 |
| ATOM | 730 | CD1 | LEU | 134 | 96.107 | 271.405 | 109.813 | 1.00 | 29.08 | 6 |
| ATOM | 731 | CD2 | LEU | 134 | 95.511 | 269.247 | 110.855 | 1.00 | 40.95 | 6 |
| ATOM | 732 | C | LEU | 134 | 92.473 | 271.758 | 111.638 | 1.00 | 42.98 | 6 |
| ATOM | 733 | O | LEU | 134 | 92.190 | 270.601 | 111.918 | 1.00 | 43.44 | 8 |
| ATOM | 734 | N | LEU | 135 | 91.711 | 272.534 | 110.864 | 1.00 | 40.09 | 7 |
| ATOM | 735 | CA | LEU | 135 | 90.421 | 272.088 | 110.317 | 1.00 | 35.41 | 6 |
| ATOM | 736 | CB | LEU | 135 | 89.432 | 273.265 | 110.279 | 1.00 | 32.99 | 6 |
| ATOM | 737 | CG | LEU | 135 | 89.161 | 274.032 | 111.582 | 1.00 | 34.85 | 6 |
| ATOM | 738 | CD1 | LEU | 135 | 90.052 | 275.216 | 111.673 | 1.00 | 36.09 | 6 |
| ATOM | 739 | CD2 | LEU | 135 | 87.742 | 274.511 | 111.652 | 1.00 | 38.95 | 6 |
| ATOM | 740 | C | LEU | 135 | 90.486 | 271.411 | 108.949 | 1.00 | 34.00 | 6 |
| ATOM | 741 | O | LEU | 135 | 91.092 | 271.953 | 108.028 | 1.00 | 33.53 | 8 |
| ATOM | 742 | N | ILE | 136 | 89.902 | 270.210 | 108.833 | 1.00 | 31.34 | 7 |
| ATOM | 743 | CA | ILE | 136 | 89.902 | 269.468 | 107.561 | 1.00 | 30.34 | 6 |
| ATOM | 744 | CB | ILE | 136 | 90.757 | 268.077 | 107.604 | 1.00 | 32.50 | 6 |
| ATOM | 745 | CG2 | ILE | 136 | 91.589 | 267.922 | 108.872 | 1.00 | 20.30 | 6 |
| ATOM | 746 | CG1 | ILE | 136 | 89.865 | 266.844 | 107.496 | 1.00 | 41.24 | 6 |
| ATOM | 747 | CD1 | ILE | 136 | 89.726 | 266.280 | 106.088 | 1.00 | 44.23 | 6 |
| ATOM | 748 | C | ILE | 136 | 88.468 | 269.305 | 107.007 | 1.00 | 28.31 | 6 |
| ATOM | 749 | O | ILE | 136 | 87.570 | 268.814 | 107.683 | 1.00 | 30.67 | 8 |
| ATOM | 750 | N | VAL | 137 | 88.247 | 269.819 | 105.804 | 1.00 | 28.22 | 7 |
| ATOM | 751 | CA | VAL | 137 | 86.938 | 269.747 | 105.153 | 1.00 | 32.67 | 6 |
| ATOM | 752 | CB | VAL | 137 | 86.570 | 271.096 | 104.463 | 1.00 | 31.29 | 6 |
| ATOM | 753 | CG1 | VAL | 137 | 85.133 | 271.052 | 103.943 | 1.00 | 32.08 | 6 |
| ATOM | 754 | CG2 | VAL | 137 | 86.755 | 272.264 | 105.429 | 1.00 | 32.62 | 6 |
| ATOM | 755 | C | VAL | 137 | 86.904 | 268.646 | 104.103 | 1.00 | 34.59 | 6 |
| ATOM | 756 | O | VAL | 137 | 87.710 | 268.660 | 103.181 | 1.00 | 37.66 | 8 |
| ATOM | 757 | N | MET | 138 | 85.943 | 267.730 | 104.206 | 1.00 | 33.28 | 7 |
| ATOM | 758 | CA | MET | 138 | 85.847 | 266.635 | 103.238 | 1.00 | 35.76 | 6 |
| ATOM | 759 | CB | MET | 138 | 86.464 | 265.370 | 103.824 | 1.00 | 37.44 | 6 |
| ATOM | 760 | CG | MET | 138 | 85.736 | 264.826 | 105.023 | 1.00 | 41.23 | 6 |
| ATOM | 761 | SD | MET | 138 | 86.916 | 264.123 | 106.117 | 1.00 | 39.58 | 16 |
| ATOM | 762 | CE | MET | 138 | 86.250 | 264.575 | 107.677 | 1.00 | 45.86 | 6 |
| ATOM | 763 | C | MET | 138 | 84.442 | 266.331 | 102.737 | 1.00 | 35.00 | 6 |
| ATOM | 764 | O | MET | 138 | 83.460 | 266.769 | 103.334 | 1.00 | 41.33 | 8 |
| ATOM | 765 | N | GLU | 139 | 84.347 | 265.540 | 101.670 | 1.00 | 30.10 | 7 |
| ATOM | 766 | CA | GLU | 139 | 83.046 | 265.193 | 101.112 | 1.00 | 39.75 | 6 |
| ATOM | 767 | CB | GLU | 139 | 83.156 | 264.290 | 99.874 | 1.00 | 42.80 | 6 |
| ATOM | 768 | CG | GLU | 139 | 83.858 | 262.953 | 100.032 | 1.00 | 48.42 | 6 |
| ATOM | 769 | CD | GLU | 139 | 83.817 | 262.138 | 98.734 | 1.00 | 54.91 | 6 |
| ATOM | 770 | OE1 | GLU | 139 | 84.549 | 262.471 | 97.770 | 1.00 | 56.45 | 8 |
| ATOM | 771 | OE2 | GLU | 139 | 83.028 | 261.176 | 98.666 | 1.00 | 60.18 | 8 |
| ATOM | 772 | C | GLU | 139 | 82.105 | 264.587 | 102.127 | 1.00 | 45.08 | 6 |
| ATOM | 773 | O | GLU | 139 | 82.426 | 263.589 | 102.761 | 1.00 | 47.19 | 8 |
| ATOM | 774 | N | CYS | 140 | 80.959 | 265.233 | 102.318 | 1.00 | 50.05 | 7 |
| ATOM | 775 | CA | CYS | 140 | 79.984 | 264.739 | 103.271 | 1.00 | 52.82 | 6 |
| ATOM | 776 | CB | CYS | 140 | 78.900 | 265.773 | 103.536 | 1.00 | 47.54 | 6 |
| ATOM | 777 | SG | CYS | 140 | 77.619 | 265.125 | 104.606 | 1.00 | 50.11 | 16 |
| ATOM | 778 | C | CYS | 140 | 79.337 | 263.422 | 102.852 | 1.00 | 55.80 | 6 |
| ATOM | 779 | O | CYS | 140 | 78.439 | 263.398 | 102.011 | 1.00 | 60.82 | 8 |
| ATOM | 780 | N | LEU | 141 | 79.807 | 262.328 | 103.439 | 1.00 | 55.40 | 7 |
| ATOM | 781 | CA | LEU | 141 | 79.269 | 261.020 | 103.133 | 1.00 | 57.23 | 6 |
| ATOM | 782 | CB | LEU | 141 | 80.332 | 259.945 | 103.322 | 1.00 | 52.66 | 6 |
| ATOM | 783 | CG | LEU | 141 | 81.637 | 260.014 | 102.538 | 1.00 | 52.11 | 6 |
| ATOM | 784 | CD1 | LEU | 141 | 82.624 | 259.085 | 103.180 | 1.00 | 53.91 | 6 |
| ATOM | 785 | CD2 | LEU | 141 | 81.432 | 259.646 | 101.096 | 1.00 | 46.92 | 6 |
| ATOM | 786 | C | LEU | 141 | 78.141 | 260.768 | 104.127 | 1.00 | 63.81 | 6 |
| ATOM | 787 | O | LEU | 141 | 78.392 | 260.546 | 105.311 | 1.00 | 63.90 | 8 |
| ATOM | 788 | N | ASP | 142 | 76.899 | 260.897 | 103.673 | 1.00 | 67.57 | 7 |
| ATOM | 789 | CA | ASP | 142 | 75.760 | 260.665 | 104.547 | 1.00 | 71.27 | 6 |
| ATOM | 790 | CB | ASP | 142 | 74.977 | 261.957 | 104.865 | 1.00 | 72.82 | 6 |
| ATOM | 791 | CG | ASP | 142 | 74.569 | 262.750 | 103.622 | 1.00 | 79.35 | 6 |
| ATOM | 792 | OD1 | ASP | 142 | 74.091 | 263.896 | 103.796 | 1.00 | 80.77 | 8 |
| ATOM | 793 | OD2 | ASP | 142 | 74.710 | 262.249 | 102.484 | 1.00 | 81.62 | 8 |
| ATOM | 794 | C | ASP | 142 | 74.884 | 259.565 | 103.957 | 1.00 | 72.26 | 6 |
| ATOM | 795 | O | ASP | 142 | 73.885 | 259.820 | 103.298 | 1.00 | 75.60 | 8 |
| ATOM | 796 | N | GLY | 143 | 75.346 | 258.333 | 104.133 | 1.00 | 72.50 | 7 |
| ATOM | 797 | CA | GLY | 143 | 74.636 | 257.169 | 103.648 | 1.00 | 70.03 | 6 |
| ATOM | 798 | C | GLY | 143 | 74.837 | 256.015 | 104.614 | 1.00 | 72.94 | 6 |
| ATOM | 799 | O | GLY | 143 | 74.685 | 254.854 | 104.236 | 1.00 | 75.97 | 8 |
| ATOM | 800 | N | GLY | 144 | 75.237 | 256.336 | 105.846 | 1.00 | 71.12 | 7 |
| ATOM | 801 | CA | GLY | 144 | 75.438 | 255.330 | 106.878 | 1.00 | 68.70 | 6 |
| ATOM | 802 | C | GLY | 144 | 76.639 | 254.410 | 106.747 | 1.00 | 68.54 | 6 |
| ATOM | 803 | O | GLY | 144 | 77.202 | 254.253 | 105.656 | 1.00 | 68.34 | 8 |
| ATOM | 804 | N | GLU | 145 | 77.021 | 253.797 | 107.870 | 1.00 | 64.95 | 7 |
| ATOM | 805 | CA | GLU | 145 | 78.147 | 252.866 | 107.912 | 1.00 | 65.01 | 6 |
| ATOM | 806 | CB | GLU | 145 | 78.490 | 252.519 | 109.355 | 1.00 | 66.21 | 6 |
| ATOM | 807 | CG | GLU | 145 | 78.773 | 253.764 | 110.201 | 1.00 | 73.19 | 6 |
| ATOM | 808 | CD | GLU | 145 | 79.216 | 253.440 | 111.628 | 1.00 | 82.81 | 6 |
| ATOM | 809 | OE1 | GLU | 145 | 79.225 | 252.218 | 112.039 | 1.00 | 83.16 | 8 |
| ATOM | 810 | OE2 | GLU | 145 | 79.581 | 254.391 | 112.420 | 1.00 | 83.36 | 8 |
| ATOM | 811 | C | GLU | 145 | 77.748 | 251.594 | 107.129 | 1.00 | 66.76 | 6 |
| ATOM | 812 | O | GLU | 145 | 76.561 | 251.245 | 107.038 | 1.00 | 68.33 | 8 |
| ATOM | 813 | N | LEU | 146 | 78.759 | 250.940 | 106.582 | 1.00 | 68.74 | 7 |
| ATOM | 814 | CA | LEU | 146 | 78.606 | 249.725 | 105.743 | 1.00 | 66.25 | 6 |
| ATOM | 815 | CB | LEU | 146 | 79.902 | 248.909 | 105.752 | 1.00 | 57.44 | 6 |
| ATOM | 816 | CG | LEU | 146 | 79.773 | 247.563 | 105.032 | 1.00 | 47.88 | 6 |
| ATOM | 817 | CD1 | LEU | 146 | 79.265 | 247.693 | 103.594 | 1.00 | 46.14 | 6 |
| ATOM | 818 | CD2 | LEU | 146 | 81.101 | 246.807 | 104.933 | 1.00 | 50.82 | 6 |
| ATOM | 819 | C | LEU | 146 | 77.465 | 248.809 | 106.229 | 1.00 | 67.39 | 6 |
| ATOM | 820 | O | LEU | 146 | 76.381 | 248.759 | 105.631 | 1.00 | 67.26 | 8 |
| ATOM | 821 | N | PHE | 147 | 77.740 | 248.090 | 107.303 | 1.00 | 70.22 | 7 |
| ATOM | 822 | CA | PHE | 147 | 76.792 | 247.116 | 107.885 | 1.00 | 72.92 | 6 |
| ATOM | 823 | CB | PHE | 147 | 77.421 | 246.452 | 109.106 | 1.00 | 65.49 | 6 |
| ATOM | 824 | CG | PHE | 147 | 78.681 | 245.679 | 108.738 | 1.00 | 66.56 | 6 |
| ATOM | 825 | CD1 | PHE | 147 | 79.865 | 245.888 | 109.451 | 1.00 | 63.92 | 6 |
| ATOM | 826 | CD2 | PHE | 147 | 78.645 | 244.768 | 107.678 | 1.00 | 62.65 | 6 |
| ATOM | 827 | CE1 | PHE | 147 | 81.022 | 245.185 | 109.099 | 1.00 | 55.36 | 6 |
| ATOM | 828 | CE2 | PHE | 147 | 79.802 | 244.066 | 107.324 | 1.00 | 60.25 | 6 |
| ATOM | 829 | CZ | PHE | 147 | 80.991 | 244.275 | 108.034 | 1.00 | 60.76 | 6 |
| ATOM | 830 | C | PHE | 147 | 75.479 | 247.787 | 108.298 | 1.00 | 74.88 | 6 |
| ATOM | 831 | O | PHE | 147 | 74.384 | 247.306 | 107.965 | 1.00 | 76.29 | 8 |
| ATOM | 832 | N | SER | 148 | 75.630 | 248.880 | 109.016 | 1.00 | 83.68 | 7 |
| ATOM | 833 | CA | SER | 148 | 74.496 | 249.666 | 109.527 | 1.00 | 84.38 | 6 |
| ATOM | 834 | CB | SER | 148 | 75.004 | 250.936 | 110.213 | 1.00 | 86.90 | 6 |
| ATOM | 835 | OG | SER | 148 | 75.821 | 250.595 | 111.322 | 1.00 | 93.14 | 8 |
| ATOM | 836 | C | SER | 148 | 73.564 | 250.083 | 108.385 | 1.00 | 83.98 | 6 |
| ATOM | 837 | O | SER | 148 | 72.724 | 250.981 | 108.543 | 1.00 | 86.96 | 8 |
| ATOM | 838 | N | ARG | 149 | 73.732 | 249.418 | 107.258 | 1.00 | 84.50 | 7 |
| ATOM | 839 | CA | ARG | 149 | 72.934 | 249.702 | 106.056 | 1.00 | 88.99 | 6 |
| ATOM | 840 | CB | ARG | 149 | 73.710 | 250.629 | 105.124 | 1.00 | 90.89 | 6 |
| ATOM | 841 | CG | ARG | 149 | 72.815 | 251.332 | 104.107 | 1.00 | 94.32 | 6 |
| ATOM | 842 | CD | ARG | 149 | 73.539 | 252.436 | 103.340 | 1.00 | 99.43 | 6 |
| ATOM | 843 | NE | ARG | 149 | 72.701 | 253.056 | 102.307 | 1.00 | 95.52 | 7 |
| ATOM | 844 | CZ | ARG | 149 | 72.814 | 252.799 | 100.999 | 1.00 | 91.72 | 6 |
| ATOM | 845 | NH1 | ARG | 149 | 73.729 | 251.934 | 100.542 | 1.00 | 93.06 | 7 |
| ATOM | 846 | NH2 | ARG | 149 | 72.048 | 253.365 | 100.057 | 1.00 | 90.70 | 7 |
| ATOM | 847 | C | ARG | 149 | 72.609 | 248.412 | 105.294 | 1.00 | 88.60 | 6 |
| ATOM | 848 | O | ARG | 149 | 71.889 | 248.432 | 104.297 | 1.00 | 89.68 | 8 |
| ATOM | 849 | N | ILE | 150 | 73.186 | 247.298 | 105.734 | 1.00 | 90.04 | 7 |
| ATOM | 850 | CA | ILE | 150 | 72.937 | 246.012 | 105.085 | 1.00 | 88.63 | 6 |
| ATOM | 851 | CB | ILE | 150 | 74.035 | 244.954 | 105.439 | 1.00 | 87.45 | 6 |
| ATOM | 852 | CG2 | ILE | 150 | 73.618 | 243.569 | 104.969 | 1.00 | 84.81 | 6 |
| ATOM | 853 | CG1 | ILE | 150 | 75.380 | 245.326 | 104.793 | 1.00 | 83.66 | 6 |
| ATOM | 854 | CD1 | ILE | 150 | 75.414 | 245.214 | 103.273 | 1.00 | 71.70 | 6 |
| ATOM | 855 | C | ILE | 150 | 71.583 | 245.566 | 105.619 | 1.00 | 87.90 | 6 |
| ATOM | 856 | O | ILE | 150 | 70.727 | 245.083 | 104.869 | 1.00 | 85.06 | 8 |
| ATOM | 857 | N | GLN | 151 | 71.389 | 245.807 | 106.914 | 1.00 | 87.71 | 7 |
| ATOM | 858 | CA | GLN | 151 | 70.155 | 245.456 | 107.609 | 1.00 | 94.70 | 6 |
| ATOM | 859 | CB | GLN | 151 | 70.393 | 245.437 | 109.123 | 1.00 | 89.92 | 6 |
| ATOM | 860 | CG | GLN | 151 | 71.027 | 246.693 | 109.681 | 1.00 | 92.02 | 6 |
| ATOM | 861 | CD | GLN | 151 | 71.391 | 246.558 | 111.147 | 1.00 | 92.48 | 6 |
| ATOM | 862 | OE1 | GLN | 151 | 70.539 | 246.254 | 111.981 | 1.00 | 91.88 | 8 |
| ATOM | 863 | NE2 | GLN | 151 | 72.663 | 246.784 | 111.468 | 1.00 | 90.51 | 7 |
| ATOM | 864 | C | GLN | 151 | 68.994 | 246.397 | 107.261 | 1.00 | 97.00 | 6 |
| ATOM | 865 | O | GLN | 151 | 67.826 | 246.067 | 107.481 | 1.00 | 99.89 | 8 |
| ATOM | 866 | N | ASP | 152 | 69.319 | 247.561 | 106.708 | 1.00 | 98.27 | 7 |
| ATOM | 867 | CA | ASP | 152 | 68.305 | 248.537 | 106.331 | 1.00 | 97.52 | 6 |
| ATOM | 868 | CB | ASP | 152 | 68.895 | 249.949 | 106.341 | 1.00 | 100.00 | 6 |
| ATOM | 869 | CG | ASP | 152 | 69.240 | 250.441 | 107.744 | 1.00 | 100.00 | 6 |
| ATOM | 870 | OD1 | ASP | 152 | 69.085 | 249.678 | 108.727 | 1.00 | 99.03 | 8 |
| ATOM | 871 | OD2 | ASP | 152 | 69.667 | 251.611 | 107.858 | 1.00 | 100.00 | 8 |
| ATOM | 872 | C | ASP | 152 | 67.706 | 248.246 | 104.959 | 1.00 | 100.00 | 6 |
| ATOM | 873 | O | ASP | 152 | 66.737 | 248.895 | 104.557 | 1.00 | 100.00 | 8 |
| ATOM | 874 | N | ALA | 153 | 68.276 | 247.265 | 104.257 | 1.00 | 100.00 | 7 |
| ATOM | 875 | CA | ALA | 153 | 67.818 | 246.870 | 102.920 | 1.00 | 100.00 | 6 |
| ATOM | 876 | CB | ALA | 153 | 68.836 | 245.920 | 102.268 | 1.00 | 100.00 | 6 |
| ATOM | 877 | C | ALA | 153 | 66.421 | 246.245 | 102.907 | 1.00 | 99.95 | 6 |
| ATOM | 878 | O | ALA | 153 | 65.495 | 246.798 | 102.312 | 1.00 | 98.31 | 8 |
| ATOM | 879 | N | GLY | 154 | 66.284 | 245.086 | 103.545 | 1.00 | 100.00 | 7 |
| ATOM | 880 | CA | GLY | 154 | 64.999 | 244.405 | 103.596 | 1.00 | 100.00 | 6 |
| ATOM | 881 | C | GLY | 154 | 64.691 | 243.532 | 102.388 | 1.00 | 100.00 | 6 |
| ATOM | 882 | O | GLY | 154 | 64.387 | 242.340 | 102.534 | 1.00 | 100.00 | 8 |
| ATOM | 883 | N | ALA | 155 | 64.737 | 244.129 | 101.199 | 1.00 | 100.00 | 7 |
| ATOM | 884 | CA | ALA | 155 | 64.466 | 243.405 | 99.960 | 1.00 | 100.00 | 6 |
| ATOM | 885 | CB | ALA | 155 | 63.096 | 243.806 | 99.401 | 1.00 | 97.77 | 6 |
| ATOM | 886 | C | ALA | 155 | 65.567 | 243.673 | 98.929 | 1.00 | 100.00 | 6 |
| ATOM | 887 | O | ALA | 155 | 65.898 | 244.864 | 98.714 | 1.00 | 100.00 | 8 |
| ATOM | 888 | OT | ALA | 155 | 66.096 | 242.688 | 98.360 | 1.00 | 100.00 | 8 |
| ATOM | 889 | CB | PHE | 158 | 72.866 | 241.609 | 100.293 | 1.00 | 100.00 | 6 |
| ATOM | 890 | CG | PHE | 158 | 74.263 | 241.800 | 100.805 | 1.00 | 98.97 | 6 |
| ATOM | 891 | CD1 | PHE | 158 | 75.327 | 241.911 | 99.926 | 1.00 | 96.55 | 6 |
| ATOM | 892 | CD2 | PHE | 158 | 74.513 | 241.866 | 102.176 | 1.00 | 98.95 | 6 |
| ATOM | 893 | CE1 | PHE | 158 | 76.619 | 242.084 | 100.399 | 1.00 | 99.47 | 6 |
| ATOM | 894 | CE2 | PHE | 158 | 75.803 | 72.954 | 243.096 | 1.00 | 100.00 | 6 |
| ATOM | 895 | CZ | PHE | 158 | 118.737 | 74.087 | 244.013 | 1.00 | 100.00 | 6 |
| ATOM | 896 | C | PHE | 158 | 118.645 | 74.381 | 244.373 | 1.00 | 99.29 | 6 |
| ATOM | 897 | O | PHE | 158 | 117.180 | 73.737 | 245.267 | 1.00 | 100.00 | 8 |
| ATOM | 898 | N | PHE | 158 | 119.440 | 72.846 | 246.032 | 1.00 | 98.27 | 7 |
| ATOM | 899 | CA | PHE | 158 | 119.059 | 74.441 | 245.460 | 1.00 | 100.00 | 6 |
| ATOM | 900 | N | THR | 159 | 120.552 | 74.237 | 246.608 | 1.00 | 99.84 | 7 |
| ATOM | 901 | CA | THR | 159 | 121.436 | 75.351 | 246.659 | 1.00 | 99.67 | 6 |
| ATOM | 902 | CB | THR | 159 | 122.482 | 74.180 | 247.927 | 1.00 | 98.54 | 6 |
| ATOM | 903 | OG1 | THR | 159 | 120.669 | 73.540 | 248.887 | 1.00 | 98.40 | 8 |
| ATOM | 904 | CG2 | THR | 159 | 121.100 | 74.853 | 247.955 | 1.00 | 98.27 | 6 |
| ATOM | 905 | C | THR | 159 | 119.528 | 74.875 | 249.138 | 1.00 | 100.00 | 6 |
| ATOM | 906 | O | THR | 159 | 118.683 | 73.475 | 249.507 | 1.00 | 100.00 | 8 |
| ATOM | 907 | N | GLU | 160 | 118.210 | 72.926 | 248.880 | 1.00 | 99.64 | 7 |
| ATOM | 908 | CA | GLU | 160 | 117.293 | 75.797 | 248.923 | 1.00 | 99.65 | 6 |
| ATOM | 909 | CB | GLU | 160 | 117.495 | 72.873 | 250.470 | 1.00 | 98.97 | 6 |
| ATOM | 910 | CG | GLU | 160 | 118.905 | 71.524 | 250.954 | 1.00 | 100.00 | 6 |
| ATOM | 911 | CD | GLU | 160 | 118.606 | 70.497 | 250.299 | 1.00 | 100.00 | 6 |
| ATOM | 912 | OE1 | GLU | 160 | 119.534 | 71.472 | 252.478 | 1.00 | 99.95 | 8 |
| ATOM | 913 | OE2 | GLU | 160 | 118.744 | 71.050 | 253.128 | 1.00 | 100.00 | 8 |
| ATOM | 914 | C | GLU | 160 | 117.767 | 71.875 | 253.007 | 1.00 | 99.48 | 6 |
| ATOM | 915 | O | GLU | 160 | 119.807 | 237.806 | 96.720 | 1.00 | 98.63 | 8 |
| ATOM | 916 | N | ARG | 161 | 77.252 | 237.544 | 95.320 | 1.00 | 100.00 | 7 |
| ATOM | 917 | CA | ARG | 161 | 77.774 | 238.422 | 94.268 | 1.00 | 99.32 | 6 |
| ATOM | 918 | CB | ARG | 161 | 76.840 | 238.487 | 93.051 | 1.00 | 99.62 | 6 |
| ATOM | 919 | CG | ARG | 161 | 76.776 | 237.243 | 92.184 | 1.00 | 99.61 | 6 |
| ATOM | 920 | CD | ARG | 161 | 76.106 | 237.519 | 90.821 | 1.00 | 100.00 | 6 |
| ATOM | 921 | NE | ARG | 161 | 74.793 | 238.165 | 90.946 | 1.00 | 100.00 | 7 |
| ATOM | 922 | CZ | ARG | 161 | 73.665 | 237.717 | 90.397 | 1.00 | 97.68 | 6 |
| ATOM | 923 | NH1 | ARG | 161 | 73.664 | 236.604 | 89.670 | 1.00 | 96.75 | 7 |
| ATOM | 924 | NH2 | ARG | 161 | 72.528 | 238.376 | 90.594 | 1.00 | 88.21 | 7 |
| ATOM | 925 | C | ARG | 161 | 77.937 | 239.835 | 94.790 | 1.00 | 99.67 | 6 |
| ATOM | 926 | O | ARG | 161 | 78.942 | 240.491 | 94.517 | 1.00 | 100.00 | 8 |
| ATOM | 927 | N | GLU | 162 | 76.923 | 240.317 | 95.502 | 1.00 | 97.59 | 7 |
| ATOM | 928 | CA | GLU | 162 | 76.963 | 241.660 | 96.056 | 1.00 | 97.16 | 6 |
| ATOM | 929 | CB | GLU | 162 | 75.601 | 242.039 | 96.626 | 1.00 | 97.68 | 6 |
| ATOM | 930 | CG | GLU | 162 | 74.510 | 242.165 | 95.578 | 1.00 | 100.00 | 6 |
| ATOM | 931 | CD | GLU | 162 | 73.129 | 242.417 | 96.180 | 1.00 | 100.00 | 6 |
| ATOM | 932 | OE1 | GLU | 162 | 73.017 | 242.543 | 97.424 | 1.00 | 100.00 | 8 |
| ATOM | 933 | OE2 | GLU | 162 | 72.149 | 242.481 | 95.399 | 1.00 | 100.00 | 8 |
| ATOM | 934 | C | GLU | 162 | 78.037 | 241.784 | 97.131 | 1.00 | 95.58 | 6 |
| ATOM | 935 | O | GLU | 162 | 78.765 | 242.782 | 97.181 | 1.00 | 97.66 | 8 |
| ATOM | 936 | N | ALA | 163 | 78.159 | 240.753 | 97.964 | 1.00 | 89.28 | 7 |
| ATOM | 937 | CA | ALA | 163 | 79.145 | 240.750 | 99.031 | 1.00 | 85.28 | 6 |
| ATOM | 938 | CB | ALA | 163 | 79.094 | 239.445 | 99.779 | 1.00 | 83.94 | 6 |
| ATOM | 939 | C | ALA | 163 | 80.523 | 240.972 | 98.435 | 1.00 | 85.18 | 6 |
| ATOM | 940 | O | ALA | 163 | 81.292 | 241.798 | 98.920 | 1.00 | 88.15 | 8 |
| ATOM | 941 | N | SER | 164 | 80.802 | 240.270 | 97.343 | 1.00 | 84.25 | 7 |
| ATOM | 942 | CA | SER | 164 | 82.082 | 240.376 | 96.655 | 1.00 | 84.10 | 6 |
| ATOM | 943 | CB | SER | 164 | 82.106 | 239.436 | 95.446 | 1.00 | 82.73 | 6 |
| ATOM | 944 | OG | SER | 164 | 83.274 | 239.620 | 94.669 | 1.00 | 81.87 | 8 |
| ATOM | 945 | C | SER | 164 | 82.365 | 241.808 | 96.203 | 1.00 | 84.15 | 6 |
| ATOM | 946 | O | SER | 164 | 83.448 | 242.342 | 96.444 | 1.00 | 86.16 | 8 |
| ATOM | 947 | N | GLU | 165 | 81.377 | 242.439 | 95.581 | 1.00 | 82.16 | 7 |
| ATOM | 948 | CA | GLU | 165 | 81.537 | 243.807 | 95.103 | 1.00 | 81.81 | 6 |
| ATOM | 949 | CB | GLU | 165 | 80.242 | 244.284 | 94.442 | 1.00 | 85.50 | 6 |
| ATOM | 950 | CG | GLU | 165 | 79.761 | 243.359 | 93.330 | 1.00 | 91.21 | 6 |
| ATOM | 951 | CD | GLU | 165 | 78.367 | 243.695 | 92.831 | 1.00 | 96.12 | 6 |
| ATOM | 952 | OE1 | GLU | 165 | 77.451 | 243.893 | 93.667 | 1.00 | 97.13 | 8 |
| ATOM | 953 | OE2 | GLU | 165 | 78.192 | 243.749 | 91.595 | 1.00 | 93.84 | 8 |
| ATOM | 954 | C | GLU | 165 | 81.968 | 244.755 | 96.225 | 1.00 | 77.89 | 6 |
| ATOM | 955 | O | GLU | 165 | 82.830 | 245.614 | 96.028 | 1.00 | 75.81 | 8 |
| ATOM | 956 | N | ILE | 166 | 81.392 | 244.571 | 97.409 | 1.00 | 73.64 | 7 |
| ATOM | 957 | CA | ILE | 166 | 81.731 | 245.414 | 98.546 | 1.00 | 70.16 | 6 |
| ATOM | 958 | CB | ILE | 166 | 80.845 | 245.092 | 99.782 | 1.00 | 71.64 | 6 |
| ATOM | 959 | CG2 | ILE | 166 | 81.350 | 245.828 | 101.032 | 1.00 | 69.95 | 6 |
| ATOM | 960 | CG1 | ILE | 166 | 79.392 | 245.478 | 99.495 | 1.00 | 71.59 | 6 |
| ATOM | 961 | CD1 | ILE | 166 | 78.467 | 245.322 | 100.691 | 1.00 | 73.87 | 6 |
| ATOM | 962 | C | ILE | 166 | 83.199 | 245.188 | 98.871 | 1.00 | 67.47 | 6 |
| ATOM | 963 | O | ILE | 166 | 83.969 | 246.134 | 99.008 | 1.00 | 65.49 | 8 |
| ATOM | 964 | N | MET | 167 | 83.599 | 243.927 | 98.931 | 1.00 | 66.41 | 7 |
| ATOM | 965 | CA | MET | 167 | 84.983 | 243.615 | 99.235 | 1.00 | 66.97 | 6 |
| ATOM | 966 | CB | MET | 167 | 85.186 | 242.114 | 99.409 | 1.00 | 64.61 | 6 |
| ATOM | 967 | CG | MET | 167 | 84.631 | 241.592 | 100.701 | 1.00 | 62.83 | 6 |
| ATOM | 968 | SD | MET | 167 | 85.047 | 242.672 | 102.095 | 1.00 | 65.77 | 16 |
| ATOM | 969 | CE | MET | 167 | 86.840 | 242.791 | 101.993 | 1.00 | 55.22 | 6 |
| ATOM | 970 | C | MET | 167 | 85.939 | 244.152 | 98.192 | 1.00 | 67.52 | 6 |
| ATOM | 971 | O | MET | 167 | 87.018 | 244.630 | 98.532 | 1.00 | 69.09 | 8 |
| ATOM | 972 | N | LYS | 168 | 85.526 | 244.121 | 96.930 | 1.00 | 66.31 | 7 |
| ATOM | 973 | CA | LYS | 168 | 86.371 | 244.611 | 95.854 | 1.00 | 67.69 | 6 |
| ATOM | 974 | CB | LYS | 168 | 85.726 | 244.329 | 94.507 | 1.00 | 64.03 | 6 |
| ATOM | 975 | CG | LYS | 168 | 86.525 | 244.820 | 93.342 | 1.00 | 59.62 | 6 |
| ATOM | 976 | CD | LYS | 168 | 85.811 | 244.534 | 92.055 | 1.00 | 62.43 | 6 |
| ATOM | 977 | CE | LYS | 168 | 86.667 | 244.936 | 90.884 | 1.00 | 65.62 | 6 |
| ATOM | 978 | NZ | LYS | 168 | 87.995 | 244.269 | 90.962 | 1.00 | 74.64 | 7 |
| ATOM | 979 | C | LYS | 168 | 86.646 | 246.101 | 96.005 | 1.00 | 72.08 | 6 |
| ATOM | 980 | O | LYS | 168 | 87.760 | 246.558 | 95.760 | 1.00 | 74.89 | 8 |
| ATOM | 981 | N | SER | 169 | 85.632 | 246.845 | 96.441 | 1.00 | 74.39 | 7 |
| ATOM | 982 | CA | SER | 169 | 85.749 | 248.289 | 96.635 | 1.00 | 74.74 | 6 |
| ATOM | 983 | CB | SER | 169 | 84.371 | 248.892 | 96.887 | 1.00 | 76.12 | 6 |
| ATOM | 984 | OG | SER | 169 | 83.470 | 248.522 | 95.859 | 1.00 | 83.04 | 8 |
| ATOM | 985 | C | SER | 169 | 86.687 | 248.606 | 97.800 | 1.00 | 73.95 | 6 |
| ATOM | 986 | O | SER | 169 | 87.665 | 249.335 | 97.625 | 1.00 | 75.56 | 8 |
| ATOM | 987 | N | ILE | 170 | 86.393 | 248.042 | 98.978 | 1.00 | 67.40 | 7 |
| ATOM | 988 | CA | ILE | 170 | 87.204 | 248.250 | 100.177 | 1.00 | 57.14 | 6 |
| ATOM | 989 | CB | ILE | 170 | 86.681 | 247.425 | 101.361 | 1.00 | 54.17 | 6 |
| ATOM | 990 | CG2 | ILE | 170 | 87.443 | 247.789 | 102.636 | 1.00 | 51.19 | 6 |
| ATOM | 991 | CG1 | ILE | 170 | 85.186 | 247.665 | 101.561 | 1.00 | 54.65 | 6 |
| ATOM | 992 | CD1 | ILE | 170 | 84.543 | 246.774 | 102.627 | 1.00 | 53.52 | 6 |
| ATOM | 993 | C | ILE | 170 | 88.618 | 247.778 | 99.867 | 1.00 | 57.07 | 6 |
| ATOM | 994 | O | ILE | 170 | 89.590 | 248.277 | 100.421 | 1.00 | 58.79 | 8 |
| ATOM | 995 | N | GLY | 171 | 88.714 | 246.796 | 98.978 | 1.00 | 55.40 | 7 |
| ATOM | 996 | CA | GLY | 171 | 90.002 | 246.275 | 98.575 | 1.00 | 56.58 | 6 |
| ATOM | 997 | C | GLY | 171 | 90.755 | 247.342 | 97.810 | 1.00 | 58.83 | 6 |
| ATOM | 998 | O | GLY | 171 | 91.868 | 247.700 | 98.189 | 1.00 | 60.19 | 8 |
| ATOM | 999 | N | GLU | 172 | 90.120 | 247.880 | 96.764 | 1.00 | 60.90 | 7 |
| ATOM | 1000 | CA | GLU | 172 | 90.696 | 248.926 | 95.910 | 1.00 | 60.95 | 6 |
| ATOM | 1001 | CB | GLU | 172 | 89.673 | 249.370 | 94.858 | 1.00 | 63.76 | 6 |
| ATOM | 1002 | CG | GLU | 172 | 89.439 | 248.329 | 93.764 | 1.00 | 74.49 | 6 |
| ATOM | 1003 | CD | GLU | 172 | 88.244 | 248.622 | 92.864 | 1.00 | 80.58 | 6 |
| ATOM | 1004 | OE1 | GLU | 172 | 87.975 | 247.791 | 91.968 | 1.00 | 81.43 | 8 |
| ATOM | 1005 | OE2 | GLU | 172 | 87.568 | 249.661 | 93.048 | 1.00 | 84.08 | 8 |
| ATOM | 1006 | C | GLU | 172 | 91.234 | 250.136 | 96.683 | 1.00 | 56.80 | 6 |
| ATOM | 1007 | O | GLU | 172 | 92.255 | 250.708 | 96.312 | 1.00 | 55.99 | 8 |
| ATOM | 1008 | N | ALA | 173 | 90.549 | 250.519 | 97.755 | 1.00 | 49.99 | 7 |
| ATOM | 1009 | CA | ALA | 173 | 90.973 | 251.647 | 98.565 | 1.00 | 46.26 | 6 |
| ATOM | 1010 | CB | ALA | 173 | 89.978 | 251.877 | 99.684 | 1.00 | 47.73 | 6 |
| ATOM | 1011 | C | ALA | 173 | 92.336 | 251.314 | 99.147 | 1.00 | 50.11 | 6 |
| ATOM | 1012 | O | ALA | 173 | 93.246 | 252.136 | 99.131 | 1.00 | 50.34 | 8 |
| ATOM | 1013 | N | ILE | 174 | 92.467 | 250.083 | 99.642 | 1.00 | 54.38 | 7 |
| ATOM | 1014 | CA | ILE | 174 | 93.707 | 249.592 | 100.246 | 1.00 | 56.54 | 6 |
| ATOM | 1015 | CB | ILE | 174 | 93.466 | 248.261 | 100.994 | 1.00 | 54.70 | 6 |
| ATOM | 1016 | CG2 | ILE | 174 | 94.729 | 247.814 | 101.677 | 1.00 | 54.75 | 6 |
| ATOM | 1017 | CG1 | ILE | 174 | 92.388 | 248.439 | 102.060 | 1.00 | 55.56 | 6 |
| ATOM | 1018 | CD1 | ILE | 174 | 92.759 | 249.439 | 103.132 | 1.00 | 61.81 | 6 |
| ATOM | 1019 | C | ILE | 174 | 94.838 | 249.430 | 99.220 | 1.00 | 55.76 | 6 |
| ATOM | 1020 | O | ILE | 174 | 96.004 | 249.721 | 99.503 | 1.00 | 54.57 | 8 |
| ATOM | 1021 | N | GLN | 175 | 94.483 | 248.972 | 98.026 | 1.00 | 50.89 | 7 |
| ATOM | 1022 | CA | GLN | 175 | 95.455 | 248.781 | 96.969 | 1.00 | 48.91 | 6 |
| ATOM | 1023 | CB | GLN | 175 | 94.782 | 248.129 | 95.768 | 1.00 | 49.16 | 6 |
| ATOM | 1024 | CG | GLN | 175 | 95.660 | 248.026 | 94.543 | 1.00 | 57.32 | 6 |
| ATOM | 1025 | CD | GLN | 175 | 95.082 | 247.080 | 93.526 | 1.00 | 67.72 | 6 |
| ATOM | 1026 | OE1 | GLN | 175 | 94.009 | 247.329 | 92.970 | 1.00 | 73.86 | 8 |
| ATOM | 1027 | NE2 | GLN | 175 | 95.771 | 245.964 | 93.296 | 1.00 | 70.28 | 7 |
| ATOM | 1028 | C | GLN | 175 | 96.109 | 250.108 | 96.574 | 1.00 | 48.06 | 6 |
| ATOM | 1029 | O | GLN | 175 | 97.296 | 250.160 | 96.265 | 1.00 | 45.44 | 8 |
| ATOM | 1030 | N | TYR | 176 | 95.326 | 251.180 | 96.590 | 1.00 | 49.18 | 7 |
| ATOM | 1031 | CA | TYR | 176 | 95.840 | 252.495 | 96.233 | 1.00 | 46.76 | 6 |
| ATOM | 1032 | CB | TYR | 176 | 94.693 | 253.492 | 96.022 | 1.00 | 47.48 | 6 |
| ATOM | 1033 | CG | TYR | 176 | 95.122 | 254.810 | 95.409 | 1.00 | 47.49 | 6 |
| ATOM | 1034 | CD1 | TYR | 176 | 94.948 | 255.049 | 94.053 | 1.00 | 47.65 | 6 |
| ATOM | 1035 | CE1 | TYR | 176 | 95.365 | 256.235 | 93.474 | 1.00 | 46.78 | 6 |
| ATOM | 1036 | CD2 | TYR | 176 | 95.728 | 255.802 | 96.179 | 1.00 | 47.35 | 6 |
| ATOM | 1037 | CE2 | TYR | 176 | 96.155 | 256.989 | 95.608 | 1.00 | 47.39 | 6 |
| ATOM | 1038 | CZ | TYR | 176 | 95.970 | 257.199 | 94.254 | 1.00 | 49.35 | 6 |
| ATOM | 1039 | OH | TYR | 176 | 96.403 | 258.366 | 93.666 | 1.00 | 55.02 | 8 |
| ATOM | 1040 | C | TYR | 176 | 96.745 | 252.951 | 97.356 | 1.00 | 44.79 | 6 |
| ATOM | 1041 | O | TYR | 176 | 97.838 | 253.460 | 97.130 | 1.00 | 42.65 | 8 |
| ATOM | 1042 | N | LEU | 177 | 96.298 | 252.714 | 98.577 | 1.00 | 45.97 | 7 |
| ATOM | 1043 | CA | LEU | 177 | 97.072 | 253.102 | 99.735 | 1.00 | 49.57 | 6 |
| ATOM | 1044 | CB | LEU | 177 | 96.256 | 252.882 | 101.004 | 1.00 | 43.02 | 6 |
| ATOM | 1045 | CG | LEU | 177 | 95.211 | 253.961 | 101.230 | 1.00 | 35.55 | 6 |
| ATOM | 1046 | CD1 | LEU | 177 | 94.448 | 253.643 | 102.471 | 1.00 | 41.43 | 6 |
| ATOM | 1047 | CD2 | LEU | 177 | 95.882 | 255.295 | 101.390 | 1.00 | 29.71 | 6 |
| ATOM | 1048 | C | LEU | 177 | 98.426 | 252.388 | 99.798 | 1.00 | 52.23 | 6 |
| ATOM | 1049 | O | LEU | 177 | 99.449 | 253.020 | 100.070 | 1.00 | 51.81 | 8 |
| ATOM | 1050 | N | HIS | 178 | 98.449 | 251.093 | 99.489 | 1.00 | 51.55 | 7 |
| ATOM | 1051 | CA | HIS | 178 | 99.710 | 250.369 | 99.532 | 1.00 | 51.08 | 6 |
| ATOM | 1052 | CB | HIS | 178 | 99.492 | 248.857 | 99.664 | 1.00 | 43.74 | 6 |
| ATOM | 1053 | CG | HIS | 178 | 98.800 | 248.456 | 100.936 | 1.00 | 47.07 | 6 |
| ATOM | 1054 | CD2 | HIS | 178 | 98.460 | 249.172 | 102.036 | 1.00 | 46.50 | 6 |
| ATOM | 1055 | ND1 | HIS | 178 | 98.328 | 247.181 | 101.158 | 1.00 | 48.56 | 7 |
| ATOM | 1056 | CE1 | HIS | 178 | 97.727 | 247.129 | 102.334 | 1.00 | 44.08 | 6 |
| ATOM | 1057 | NE2 | HIS | 178 | 97.792 | 248.324 | 102.886 | 1.00 | 39.34 | 7 |
| ATOM | 1058 | C | HIS | 178 | 100.592 | 250.728 | 98.337 | 1.00 | 54.56 | 6 |
| ATOM | 1059 | O | HIS | 178 | 101.808 | 250.857 | 98.477 | 1.00 | 60.07 | 8 |
| ATOM | 1060 | N | SER | 179 | 99.977 | 251.005 | 97.190 | 1.00 | 53.28 | 7 |
| ATOM | 1061 | CA | SER | 179 | 100.742 | 251.358 | 95.999 | 1.00 | 48.40 | 6 |
| ATOM | 1062 | CB | SER | 179 | 99.839 | 251.443 | 94.792 | 1.00 | 46.50 | 6 |
| ATOM | 1063 | OG | SER | 179 | 98.933 | 252.510 | 94.955 | 1.00 | 58.97 | 8 |
| ATOM | 1064 | C | SER | 179 | 101.491 | 252.669 | 96.173 | 1.00 | 50.14 | 6 |
| ATOM | 1065 | O | SER | 179 | 102.375 | 252.984 | 95.383 | 1.00 | 55.19 | 8 |
| ATOM | 1066 | N | ILE | 180 | 101.088 | 253.476 | 97.147 | 1.00 | 48.95 | 7 |
| ATOM | 1067 | CA | ILE | 180 | 101.776 | 254.740 | 97.372 | 1.00 | 49.17 | 6 |
| ATOM | 1068 | CB | ILE | 180 | 100.885 | 256.001 | 97.168 | 1.00 | 45.91 | 6 |
| ATOM | 1069 | CG2 | ILE | 180 | 100.692 | 256.258 | 95.696 | 1.00 | 49.21 | 6 |
| ATOM | 1070 | CG1 | ILE | 180 | 99.542 | 255.878 | 97.875 | 1.00 | 46.39 | 6 |
| ATOM | 1071 | CD1 | ILE | 180 | 98.718 | 257.152 | 97.806 | 1.00 | 40.29 | 6 |
| ATOM | 1072 | C | ILE | 180 | 102.478 | 254.744 | 98.726 | 1.00 | 51.29 | 6 |
| ATOM | 1073 | O | ILE | 180 | 102.776 | 255.802 | 99.285 | 1.00 | 51.35 | 8 |
| ATOM | 1074 | N | ASN | 181 | 102.737 | 253.532 | 99.226 | 1.00 | 52.87 | 7 |
| ATOM | 1075 | CA | ASN | 181 | 103.412 | 253.285 | 100.502 | 1.00 | 57.36 | 6 |
| ATOM | 1076 | CB | ASN | 181 | 104.861 | 253.769 | 100.424 | 1.00 | 63.31 | 6 |
| ATOM | 1077 | CG | ASN | 181 | 105.620 | 253.120 | 99.295 | 1.00 | 75.30 | 6 |
| ATOM | 1078 | OD1 | ASN | 181 | 105.802 | 251.901 | 99.276 | 1.00 | 83.57 | 8 |
| ATOM | 1079 | ND2 | ASN | 181 | 106.040 | 253.924 | 98.324 | 1.00 | 79.92 | 7 |
| ATOM | 1080 | C | ASN | 181 | 102.743 | 253.872 | 101.735 | 1.00 | 57.83 | 6 |
| ATOM | 1081 | O | ASN | 181 | 103.406 | 254.484 | 102.582 | 1.00 | 61.51 | 8 |
| ATOM | 1082 | N | ILE | 182 | 101.437 | 253.654 | 101.849 | 1.00 | 51.41 | 7 |
| ATOM | 1083 | CA | ILE | 182 | 100.667 | 254.159 | 102.974 | 1.00 | 46.59 | 6 |
| ATOM | 1084 | CB | ILE | 182 | 99.690 | 255.300 | 102.533 | 1.00 | 44.22 | 6 |
| ATOM | 1085 | CG2 | ILE | 182 | 98.816 | 255.745 | 103.693 | 1.00 | 40.74 | 6 |
| ATOM | 1086 | CG1 | ILE | 182 | 100.458 | 256.506 | 101.992 | 1.00 | 40.60 | 6 |
| ATOM | 1087 | CD1 | ILE | 182 | 99.577 | 257.562 | 101.394 | 1.00 | 29.78 | 6 |
| ATOM | 1088 | C | ILE | 182 | 99.831 | 253.034 | 103.551 | 1.00 | 48.39 | 6 |
| ATOM | 1089 | O | ILE | 182 | 99.079 | 252.379 | 102.832 | 1.00 | 49.44 | 8 |
| ATOM | 1090 | N | ALA | 183 | 100.015 | 252.764 | 104.834 | 1.00 | 46.67 | 7 |
| ATOM | 1091 | CA | ALA | 183 | 99.255 | 251.719 | 105.497 | 1.00 | 47.68 | 6 |
| ATOM | 1092 | CB | ALA | 183 | 100.129 | 251.001 | 106.454 | 1.00 | 54.24 | 6 |
| ATOM | 1093 | C | ALA | 183 | 98.143 | 252.462 | 106.240 | 1.00 | 49.64 | 6 |
| ATOM | 1094 | O | ALA | 183 | 98.410 | 253.427 | 106.960 | 1.00 | 52.17 | 8 |
| ATOM | 1095 | N | HIS | 184 | 96.901 | 252.036 | 106.075 | 1.00 | 47.11 | 7 |
| ATOM | 1096 | CA | HIS | 184 | 95.812 | 252.724 | 106.757 | 1.00 | 50.78 | 6 |
| ATOM | 1097 | CB | HIS | 184 | 94.443 | 252.293 | 106.216 | 1.00 | 55.29 | 6 |
| ATOM | 1098 | CG | HIS | 184 | 93.301 | 253.092 | 106.765 | 1.00 | 58.16 | 6 |
| ATOM | 1099 | CD2 | HIS | 184 | 92.997 | 254.406 | 106.647 | 1.00 | 60.55 | 6 |
| ATOM | 1100 | ND1 | HIS | 184 | 92.312 | 252.541 | 107.553 | 1.00 | 61.00 | 7 |
| ATOM | 1101 | CE1 | HIS | 184 | 91.447 | 253.478 | 107.895 | 1.00 | 57.28 | 6 |
| ATOM | 1102 | NE2 | HIS | 184 | 91.839 | 254.618 | 107.358 | 1.00 | 61.01 | 7 |
| ATOM | 1103 | C | HIS | 184 | 95.859 | 252.567 | 108.264 | 1.00 | 49.24 | 6 |
| ATOM | 1104 | O | HIS | 184 | 95.921 | 253.559 | 108.978 | 1.00 | 51.74 | 8 |
| ATOM | 1105 | N | ARG | 185 | 95.806 | 251.320 | 108.728 | 1.00 | 48.51 | 7 |
| ATOM | 1106 | CA | ARG | 185 | 95.841 | 250.980 | 110.147 | 1.00 | 47.17 | 6 |
| ATOM | 1107 | CB | ARG | 185 | 97.055 | 251.629 | 110.815 | 1.00 | 46.44 | 6 |
| ATOM | 1108 | CG | ARG | 185 | 98.341 | 251.272 | 110.142 | 1.00 | 51.52 | 6 |
| ATOM | 1109 | CD | ARG | 185 | 99.449 | 251.153 | 111.127 | 1.00 | 53.05 | 6 |
| ATOM | 1110 | NE | ARG | 185 | 100.278 | 252.341 | 111.191 | 1.00 | 53.65 | 7 |
| ATOM | 1111 | CZ | ARG | 185 | 100.571 | 252.958 | 112.326 | 1.00 | 60.18 | 6 |
| ATOM | 1112 | NH1 | ARG | 185 | 100.075 | 252.487 | 113.462 | 1.00 | 61.35 | 7 |
| ATOM | 1113 | NH2 | ARG | 185 | 101.414 | 253.988 | 112.339 | 1.00 | 61.75 | 7 |
| ATOM | 1114 | C | ARG | 185 | 94.565 | 251.273 | 110.957 | 1.00 | 51.20 | 6 |
| ATOM | 1115 | O | ARG | 185 | 94.628 | 251.567 | 112.155 | 1.00 | 56.09 | 8 |
| ATOM | 1116 | N | ASP | 186 | 93.404 | 251.212 | 110.318 | 1.00 | 48.72 | 7 |
| ATOM | 1117 | CA | ASP | 186 | 92.172 | 251.467 | 111.034 | 1.00 | 40.68 | 6 |
| ATOM | 1118 | CB | ASP | 186 | 92.085 | 252.915 | 111.479 | 1.00 | 44.16 | 6 |
| ATOM | 1119 | CG | ASP | 186 | 91.420 | 253.059 | 112.834 | 1.00 | 51.99 | 6 |
| ATOM | 1120 | OD1 | ASP | 186 | 90.682 | 254.043 | 113.061 | 1.00 | 53.20 | 8 |
| ATOM | 1121 | OD2 | ASP | 186 | 91.652 | 252.178 | 113.686 | 1.00 | 58.97 | 8 |
| ATOM | 1122 | C | ASP | 186 | 90.960 | 251.100 | 110.226 | 1.00 | 41.38 | 6 |
| ATOM | 1123 | O | ASP | 186 | 89.914 | 251.711 | 110.363 | 1.00 | 43.75 | 8 |
| ATOM | 1124 | N | VAL | 187 | 91.085 | 250.064 | 109.411 | 1.00 | 39.20 | 7 |
| ATOM | 1125 | CA | VAL | 187 | 89.976 | 249.623 | 108.583 | 1.00 | 39.69 | 6 |
| ATOM | 1126 | CB | VAL | 187 | 90.487 | 248.745 | 107.433 | 1.00 | 36.87 | 6 |
| ATOM | 1127 | CG1 | VAL | 187 | 89.342 | 248.258 | 106.588 | 1.00 | 32.54 | 6 |
| ATOM | 1128 | CG2 | VAL | 187 | 91.477 | 249.519 | 106.592 | 1.00 | 30.39 | 6 |
| ATOM | 1129 | C | VAL | 187 | 88.881 | 248.899 | 109.377 | 1.00 | 43.45 | 6 |
| ATOM | 1130 | O | VAL | 187 | 88.689 | 247.695 | 109.229 | 1.00 | 45.72 | 8 |
| ATOM | 1131 | N | LYS | 188 | 88.183 | 249.646 | 110.232 | 1.00 | 47.30 | 7 |
| ATOM | 1132 | CA | LYS | 188 | 87.100 | 249.107 | 111.057 | 1.00 | 51.33 | 6 |
| ATOM | 1133 | CB | LYS | 188 | 86.867 | 249.957 | 112.307 | 1.00 | 49.13 | 6 |
| ATOM | 1134 | CG | LYS | 188 | 88.086 | 250.443 | 113.038 | 1.00 | 55.63 | 6 |
| ATOM | 1135 | CD | LYS | 188 | 87.711 | 251.686 | 113.831 | 1.00 | 58.48 | 6 |
| ATOM | 1136 | CE | LYS | 188 | 88.841 | 252.183 | 114.711 | 1.00 | 57.13 | 6 |
| ATOM | 1137 | NZ | LYS | 188 | 88.737 | 253.666 | 114.966 | 1.00 | 58.82 | 7 |
| ATOM | 1138 | C | LYS | 188 | 85.830 | 249.244 | 110.237 | 1.00 | 56.93 | 6 |
| ATOM | 1139 | O | LYS | 188 | 85.808 | 249.931 | 109.210 | 1.00 | 58.62 | 8 |
| ATOM | 1140 | N | PRO | 189 | 84.750 | 248.585 | 110.674 | 1.00 | 61.62 | 7 |
| ATOM | 1141 | CD | PRO | 189 | 84.717 | 247.481 | 111.647 | 1.00 | 65.55 | 6 |
| ATOM | 1142 | CA | PRO | 189 | 83.481 | 248.678 | 109.949 | 1.00 | 62.65 | 6 |
| ATOM | 1143 | CB | PRO | 189 | 82.573 | 247.754 | 110.751 | 1.00 | 62.24 | 6 |
| ATOM | 1144 | CG | PRO | 189 | 83.513 | 246.685 | 111.188 | 1.00 | 67.01 | 6 |
| ATOM | 1145 | C | PRO | 189 | 82.998 | 250.116 | 110.031 | 1.00 | 62.95 | 6 |
| ATOM | 1146 | O | PRO | 189 | 82.466 | 250.665 | 109.072 | 1.00 | 65.70 | 8 |
| ATOM | 1147 | N | GLU | 190 | 83.270 | 250.740 | 111.171 | 1.00 | 65.71 | 7 |
| ATOM | 1148 | CA | GLU | 190 | 82.876 | 252.123 | 111.421 | 1.00 | 67.33 | 6 |
| ATOM | 1149 | CB | GLU | 190 | 83.208 | 252.521 | 112.873 | 1.00 | 71.23 | 6 |
| ATOM | 1150 | CG | GLU | 190 | 82.547 | 251.611 | 113.955 | 1.00 | 84.17 | 6 |
| ATOM | 1151 | CD | GLU | 190 | 83.380 | 250.358 | 114.350 | 1.00 | 88.04 | 6 |
| ATOM | 1152 | OE1 | GLU | 190 | 84.187 | 250.431 | 115.310 | 1.00 | 86.76 | 8 |
| ATOM | 1153 | OE2 | GLU | 190 | 83.207 | 249.288 | 113.727 | 1.00 | 87.85 | 8 |
| ATOM | 1154 | C | GLU | 190 | 83.471 | 253.127 | 110.422 | 1.00 | 64.28 | 6 |
| ATOM | 1155 | O | GLU | 190 | 82.783 | 254.047 | 109.982 | 1.00 | 66.82 | 8 |
| ATOM | 1156 | N | ASN | 191 | 84.715 | 252.906 | 110.003 | 1.00 | 56.39 | 7 |
| ATOM | 1157 | CA | ASN | 191 | 85.357 | 253.812 | 109.060 | 1.00 | 45.18 | 6 |
| ATOM | 1158 | CB | ASN | 191 | 86.867 | 253.793 | 109.221 | 1.00 | 42.98 | 6 |
| ATOM | 1159 | CG | ASN | 191 | 87.309 | 254.354 | 110.536 | 1.00 | 49.63 | 6 |
| ATOM | 1160 | OD1 | ASN | 191 | 86.626 | 255.187 | 111.123 | 1.00 | 60.26 | 8 |
| ATOM | 1161 | ND2 | ASN | 191 | 88.460 | 253.908 | 111.012 | 1.00 | 51.71 | 7 |
| ATOM | 1162 | C | ASN | 191 | 85.005 | 253.616 | 107.611 | 1.00 | 44.46 | 6 |
| ATOM | 1163 | O | ASN | 191 | 85.763 | 254.043 | 106.737 | 1.00 | 41.99 | 8 |
| ATOM | 1164 | N | LEU | 192 | 83.888 | 252.942 | 107.349 | 1.00 | 41.14 | 7 |
| ATOM | 1165 | CA | LEU | 192 | 83.453 | 252.708 | 105.969 | 1.00 | 48.59 | 6 |
| ATOM | 1166 | CB | LEU | 192 | 83.408 | 251.218 | 105.646 | 1.00 | 48.59 | 6 |
| ATOM | 1167 | CG | LEU | 192 | 84.743 | 250.479 | 105.724 | 1.00 | 49.22 | 6 |
| ATOM | 1168 | CD1 | LEU | 192 | 84.472 | 249.000 | 105.613 | 1.00 | 52.37 | 6 |
| ATOM | 1169 | CD2 | LEU | 192 | 85.695 | 250.933 | 104.632 | 1.00 | 40.17 | 6 |
| ATOM | 1170 | C | LEU | 192 | 82.075 | 253.349 | 105.800 | 1.00 | 49.36 | 6 |
| ATOM | 1171 | O | LEU | 192 | 81.088 | 252.868 | 106.354 | 1.00 | 55.53 | 8 |
| ATOM | 1172 | N | LEU | 193 | 82.016 | 254.458 | 105.066 | 1.00 | 46.44 | 7 |
| ATOM | 1173 | CA | LEU | 193 | 80.760 | 255.152 | 104.855 | 1.00 | 40.87 | 6 |
| ATOM | 1174 | CB | LEU | 193 | 80.890 | 256.568 | 105.363 | 1.00 | 36.02 | 6 |
| ATOM | 1175 | CG | LEU | 193 | 81.582 | 256.608 | 106.719 | 1.00 | 33.27 | 6 |
| ATOM | 1176 | CD1 | LEU | 193 | 82.047 | 257.996 | 107.073 | 1.00 | 41.39 | 6 |
| ATOM | 1177 | CD2 | LEU | 193 | 80.631 | 256.094 | 107.738 | 1.00 | 39.25 | 6 |
| ATOM | 1178 | C | LEU | 193 | 80.336 | 255.178 | 103.412 | 1.00 | 47.34 | 6 |
| ATOM | 1179 | O | LEU | 193 | 81.156 | 255.270 | 102.506 | 1.00 | 48.69 | 8 |
| ATOM | 1180 | N | TYR | 194 | 79.032 | 255.073 | 103.211 | 1.00 | 52.17 | 7 |
| ATOM | 1181 | CA | TYR | 194 | 78.436 | 255.080 | 101.884 | 1.00 | 57.65 | 6 |
| ATOM | 1182 | CB | TYR | 194 | 77.139 | 254.266 | 101.929 | 1.00 | 60.06 | 6 |
| ATOM | 1183 | CG | TYR | 194 | 77.275 | 252.840 | 101.431 | 1.00 | 59.19 | 6 |
| ATOM | 1184 | CD1 | TYR | 194 | 77.639 | 252.588 | 100.113 | 1.00 | 56.00 | 6 |
| ATOM | 1185 | CE1 | TYR | 194 | 77.733 | 251.308 | 99.626 | 1.00 | 56.23 | 6 |
| ATOM | 1186 | CD2 | TYR | 194 | 77.006 | 251.752 | 102.259 | 1.00 | 55.91 | 6 |
| ATOM | 1187 | CE2 | TYR | 194 | 77.096 | 250.454 | 101.777 | 1.00 | 58.61 | 6 |
| ATOM | 1188 | CZ | TYR | 194 | 77.462 | 250.241 | 100.451 | 1.00 | 62.12 | 6 |
| ATOM | 1189 | OH | TYR | 194 | 77.553 | 248.968 | 99.922 | 1.00 | 67.45 | 8 |
| ATOM | 1190 | C | TYR | 194 | 78.163 | 256.546 | 101.550 | 1.00 | 60.39 | 6 |
| ATOM | 1191 | O | TYR | 194 | 77.766 | 257.310 | 102.435 | 1.00 | 62.81 | 8 |
| ATOM | 1192 | N | THR | 195 | 78.377 | 256.959 | 100.303 | 1.00 | 60.44 | 7 |
| ATOM | 1193 | CA | THR | 195 | 78.134 | 258.361 | 99.954 | 1.00 | 63.96 | 6 |
| ATOM | 1194 | CB | THR | 195 | 78.442 | 258.669 | 98.481 | 1.00 | 58.05 | 6 |
| ATOM | 1195 | OG1 | THR | 195 | 77.790 | 257.713 | 97.647 | 1.00 | 59.81 | 8 |
| ATOM | 1196 | CG2 | THR | 195 | 79.926 | 258.649 | 98.216 | 1.00 | 55.74 | 6 |
| ATOM | 1197 | C | THR | 195 | 76.720 | 258.846 | 100.266 | 1.00 | 71.40 | 6 |
| ATOM | 1198 | O | THR | 195 | 76.510 | 259.594 | 101.221 | 1.00 | 76.25 | 8 |
| ATOM | 1199 | N | SER | 196 | 75.756 | 258.403 | 99.468 | 1.00 | 74.19 | 7 |
| ATOM | 1200 | CA | SER | 196 | 74.363 | 258.782 | 99.639 | 1.00 | 75.97 | 6 |
| ATOM | 1201 | CB | SER | 196 | 73.807 | 259.245 | 98.300 | 1.00 | 78.14 | 6 |
| ATOM | 1202 | OG | SER | 196 | 74.475 | 258.593 | 97.234 | 1.00 | 80.44 | 8 |
| ATOM | 1203 | C | SER | 196 | 73.545 | 257.627 | 100.186 | 1.00 | 79.13 | 6 |
| ATOM | 1204 | O | SER | 196 | 74.105 | 256.592 | 100.543 | 1.00 | 80.00 | 8 |
| ATOM | 1205 | N | ALA | 197 | 72.228 | 257.820 | 100.288 | 1.00 | 83.31 | 7 |
| ATOM | 1206 | CA | ALA | 197 | 71.320 | 256.784 | 100.802 | 1.00 | 84.85 | 6 |
| ATOM | 1207 | CB | ALA | 197 | 70.150 | 257.417 | 101.561 | 1.00 | 75.89 | 6 |
| ATOM | 1208 | C | ALA | 197 | 70.806 | 255.890 | 99.676 | 1.00 | 86.03 | 6 |
| ATOM | 1209 | O | ALA | 197 | 70.322 | 254.788 | 99.922 | 1.00 | 85.29 | 8 |
| ATOM | 1210 | N | ARG | 198 | 70.966 | 256.368 | 98.445 | 1.00 | 89.96 | 7 |
| ATOM | 1211 | CA | ARG | 198 | 70.542 | 255.667 | 97.230 | 1.00 | 95.64 | 6 |
| ATOM | 1212 | CB | ARG | 198 | 70.782 | 256.584 | 96.016 | 1.00 | 98.85 | 6 |
| ATOM | 1213 | CG | ARG | 198 | 72.208 | 257.104 | 95.879 | 1.00 | 100.00 | 6 |
| ATOM | 1214 | CD | ARG | 198 | 72.355 | 258.073 | 94.709 | 1.00 | 100.00 | 6 |
| ATOM | 1215 | NE | ARG | 198 | 72.564 | 257.391 | 93.432 | 1.00 | 100.00 | 7 |
| ATOM | 1216 | CZ | ARG | 198 | 72.521 | 257.987 | 92.241 | 1.00 | 100.00 | 6 |
| ATOM | 1217 | NH1 | ARG | 198 | 72.269 | 259.291 | 92.144 | 1.00 | 100.00 | 7 |
| ATOM | 1218 | NH2 | ARG | 198 | 72.745 | 257.275 | 91.142 | 1.00 | 100.00 | 7 |
| ATOM | 1219 | C | ARG | 198 | 71.222 | 254.298 | 97.024 | 1.00 | 96.37 | 6 |
| ATOM | 1220 | O | ARG | 198 | 72.057 | 253.891 | 97.831 | 1.00 | 96.91 | 8 |
| ATOM | 1221 | N | PRO | 199 | 70.839 | 253.554 | 95.963 | 1.00 | 96.90 | 7 |
| ATOM | 1222 | CD | PRO | 199 | 69.736 | 253.830 | 95.020 | 1.00 | 96.34 | 6 |
| ATOM | 1223 | CA | PRO | 199 | 71.434 | 252.237 | 95.685 | 1.00 | 96.10 | 6 |
| ATOM | 1224 | CB | PRO | 199 | 70.446 | 251.629 | 94.694 | 1.00 | 96.27 | 6 |
| ATOM | 1225 | CG | PRO | 199 | 69.994 | 252.831 | 93.913 | 1.00 | 96.20 | 6 |
| ATOM | 1226 | C | PRO | 199 | 72.841 | 252.319 | 95.076 | 1.00 | 94.70 | 6 |
| ATOM | 1227 | O | PRO | 199 | 73.753 | 251.601 | 95.487 | 1.00 | 94.16 | 8 |
| ATOM | 1228 | N | ASN | 200 | 73.006 | 253.202 | 94.096 | 1.00 | 92.92 | 7 |
| ATOM | 1229 | CA | ASN | 200 | 74.283 | 253.393 | 93.421 | 1.00 | 91.48 | 6 |
| ATOM | 1230 | CB | ASN | 200 | 74.101 | 254.336 | 92.220 | 1.00 | 97.04 | 6 |
| ATOM | 1231 | CG | ASN | 200 | 75.391 | 254.558 | 91.440 | 1.00 | 100.00 | 6 |
| ATOM | 1232 | OD1 | ASN | 200 | 76.085 | 255.561 | 91.632 | 1.00 | 100.00 | 8 |
| ATOM | 1233 | ND2 | ASN | 200 | 75.718 | 253.617 | 90.556 | 1.00 | 100.00 | 7 |
| ATOM | 1234 | C | ASN | 200 | 75.329 | 253.957 | 94.383 | 1.00 | 87.10 | 6 |
| ATOM | 1235 | O | ASN | 200 | 76.527 | 253.908 | 94.104 | 1.00 | 85.57 | 8 |
| ATOM | 1236 | N | ALA | 201 | 74.867 | 254.484 | 95.516 | 1.00 | 82.18 | 7 |
| ATOM | 1237 | CA | ALA | 201 | 75.748 | 255.060 | 96.532 | 1.00 | 78.87 | 6 |
| ATOM | 1238 | CB | ALA | 201 | 75.003 | 255.212 | 97.842 | 1.00 | 79.82 | 6 |
| ATOM | 1239 | C | ALA | 201 | 76.989 | 254.214 | 96.745 | 1.00 | 74.77 | 6 |
| ATOM | 1240 | O | ALA | 201 | 76.894 | 253.056 | 97.142 | 1.00 | 76.13 | 8 |
| ATOM | 1241 | N | ILE | 202 | 78.150 | 254.783 | 96.447 | 1.00 | 69.27 | 7 |
| ATOM | 1242 | CA | ILE | 202 | 79.392 | 254.053 | 96.615 | 1.00 | 67.80 | 6 |
| ATOM | 1243 | CB | ILE | 202 | 80.327 | 254.323 | 95.452 | 1.00 | 67.42 | 6 |
| ATOM | 1244 | CG2 | ILE | 202 | 80.728 | 255.767 | 95.422 | 1.00 | 56.12 | 6 |
| ATOM | 1245 | CG1 | ILE | 202 | 81.502 | 253.340 | 95.510 | 1.00 | 79.53 | 6 |
| ATOM | 1246 | CD1 | ILE | 202 | 81.087 | 251.827 | 95.400 | 1.00 | 74.54 | 6 |
| ATOM | 1247 | C | ILE | 202 | 80.092 | 254.286 | 97.965 | 1.00 | 65.15 | 6 |
| ATOM | 1248 | O | ILE | 202 | 80.070 | 255.390 | 98.498 | 1.00 | 66.53 | 8 |
| ATOM | 1249 | N | LEU | 203 | 80.666 | 253.230 | 98.545 | 1.00 | 61.06 | 7 |
| ATOM | 1250 | CA | LEU | 203 | 81.339 | 253.381 | 99.829 | 1.00 | 56.16 | 6 |
| ATOM | 1251 | CB | LEU | 203 | 81.108 | 252.186 | 100.772 | 1.00 | 58.37 | 6 |
| ATOM | 1252 | CG | LEU | 203 | 81.307 | 250.698 | 100.456 | 1.00 | 56.67 | 6 |
| ATOM | 1253 | CD1 | LEU | 203 | 82.537 | 250.450 | 99.606 | 1.00 | 61.89 | 6 |
| ATOM | 1254 | CD2 | LEU | 203 | 81.390 | 249.932 | 101.774 | 1.00 | 49.61 | 6 |
| ATOM | 1255 | C | LEU | 203 | 82.807 | 253.716 | 99.767 | 1.00 | 51.96 | 6 |
| ATOM | 1256 | O | LEU | 203 | 83.535 | 253.263 | 98.882 | 1.00 | 49.35 | 8 |
| ATOM | 1257 | N | LYS | 204 | 83.234 | 254.509 | 100.740 | 1.00 | 45.12 | 7 |
| ATOM | 1258 | CA | LYS | 204 | 84.611 | 254.922 | 100.819 | 1.00 | 43.06 | 6 |
| ATOM | 1259 | CB | LYS | 204 | 84.790 | 256.344 | 100.284 | 1.00 | 42.02 | 6 |
| ATOM | 1260 | CG | LYS | 204 | 83.511 | 257.097 | 99.994 | 1.00 | 45.94 | 6 |
| ATOM | 1261 | CD | LYS | 204 | 83.421 | 257.543 | 98.535 | 1.00 | 44.24 | 6 |
| ATOM | 1262 | CE | LYS | 204 | 84.528 | 258.498 | 98.155 | 1.00 | 41.72 | 6 |
| ATOM | 1263 | NZ | LYS | 204 | 84.277 | 259.032 | 96.814 | 1.00 | 39.77 | 7 |
| ATOM | 1264 | C | LYS | 204 | 85.142 | 254.815 | 102.228 | 1.00 | 42.06 | 6 |
| ATOM | 1265 | O | LYS | 204 | 84.409 | 254.988 | 103.200 | 1.00 | 40.61 | 8 |
| ATOM | 1266 | N | LEU | 205 | 86.422 | 254.472 | 102.313 | 1.00 | 38.25 | 7 |
| ATOM | 1267 | CA | LEU | 205 | 87.121 | 254.317 | 103.569 | 1.00 | 40.08 | 6 |
| ATOM | 1268 | CB | LEU | 205 | 88.347 | 253.440 | 103.345 | 1.00 | 42.57 | 6 |
| ATOM | 1269 | CG | LEU | 205 | 89.348 | 253.319 | 104.490 | 1.00 | 39.94 | 6 |
| ATOM | 1270 | CD1 | LEU | 205 | 88.971 | 252.120 | 105.338 | 1.00 | 38.09 | 6 |
| ATOM | 1271 | CD2 | LEU | 205 | 90.755 | 253.190 | 103.915 | 1.00 | 39.78 | 6 |
| ATOM | 1272 | C | LEU | 205 | 87.562 | 255.681 | 104.064 | 1.00 | 40.59 | 6 |
| ATOM | 1273 | O | LEU | 205 | 88.048 | 256.502 | 103.281 | 1.00 | 44.38 | 8 |
| ATOM | 1274 | N | THR | 206 | 87.438 | 255.905 | 105.370 | 1.00 | 39.00 | 7 |
| ATOM | 1275 | CA | THR | 206 | 87.830 | 257.180 | 105.959 | 1.00 | 38.13 | 6 |
| ATOM | 1276 | CB | THR | 206 | 86.589 | 257.967 | 106.448 | 1.00 | 35.42 | 6 |
| ATOM | 1277 | OG1 | THR | 206 | 86.148 | 257.423 | 107.695 | 1.00 | 42.69 | 8 |
| ATOM | 1278 | CG2 | THR | 206 | 85.452 | 257.867 | 105.465 | 1.00 | 37.41 | 6 |
| ATOM | 1279 | C | THR | 206 | 88.750 | 257.006 | 107.170 | 1.00 | 37.92 | 6 |
| ATOM | 1280 | O | THR | 206 | 89.062 | 255.897 | 107.572 | 1.00 | 45.69 | 8 |
| ATOM | 1281 | N | ASP | 207 | 89.181 | 258.130 | 107.725 | 1.00 | 36.48 | 7 |
| ATOM | 1282 | CA | ASP | 207 | 90.046 | 258.193 | 108.891 | 1.00 | 35.51 | 6 |
| ATOM | 1283 | CB | ASP | 207 | 89.394 | 257.479 | 110.065 | 1.00 | 38.57 | 6 |
| ATOM | 1284 | CG | ASP | 207 | 89.933 | 257.955 | 111.402 | 1.00 | 47.47 | 6 |
| ATOM | 1285 | OD1 | ASP | 207 | 90.957 | 258.676 | 111.442 | 1.00 | 46.47 | 8 |
| ATOM | 1286 | OD2 | ASP | 207 | 89.322 | 257.619 | 112.424 | 1.00 | 43.26 | 8 |
| ATOM | 1287 | C | ASP | 207 | 91.508 | 257.800 | 108.799 | 1.00 | 36.22 | 6 |
| ATOM | 1288 | O | ASP | 207 | 91.910 | 256.715 | 109.203 | 1.00 | 43.91 | 8 |
| ATOM | 1289 | N | PHE | 208 | 92.326 | 258.761 | 108.420 | 1.00 | 33.81 | 7 |
| ATOM | 1290 | CA | PHE | 208 | 93.747 | 258.525 | 108.294 | 1.00 | 29.95 | 6 |
| ATOM | 1291 | CB | PHE | 208 | 94.287 | 259.273 | 107.066 | 1.00 | 28.44 | 6 |
| ATOM | 1292 | CG | PHE | 208 | 93.917 | 258.626 | 105.768 | 1.00 | 28.85 | 6 |
| ATOM | 1293 | CD1 | PHE | 208 | 92.582 | 258.410 | 105.434 | 1.00 | 27.89 | 6 |
| ATOM | 1294 | CD2 | PHE | 208 | 94.904 | 258.169 | 104.909 | 1.00 | 33.81 | 6 |
| ATOM | 1295 | CE1 | PHE | 208 | 92.239 | 257.745 | 104.260 | 1.00 | 38.94 | 6 |
| ATOM | 1296 | CE2 | PHE | 208 | 94.575 | 257.496 | 103.721 | 1.00 | 38.70 | 6 |
| ATOM | 1297 | CZ | PHE | 208 | 93.243 | 257.281 | 103.397 | 1.00 | 41.48 | 6 |
| ATOM | 1298 | C | PHE | 208 | 94.444 | 258.926 | 109.586 | 1.00 | 29.60 | 6 |
| ATOM | 1299 | O | PHE | 208 | 95.592 | 259.408 | 109.588 | 1.00 | 30.80 | 8 |
| ATOM | 1300 | N | GLY | 209 | 93.753 | 258.666 | 110.691 | 1.00 | 29.35 | 7 |
| ATOM | 1301 | CA | GLY | 209 | 94.285 | 259.017 | 111.991 | 1.00 | 35.84 | 6 |
| ATOM | 1302 | C | GLY | 209 | 95.396 | 258.094 | 112.433 | 1.00 | 40.83 | 6 |
| ATOM | 1303 | O | GLY | 209 | 96.220 | 258.457 | 113.258 | 1.00 | 39.24 | 8 |
| ATOM | 1304 | N | PHE | 210 | 95.420 | 256.886 | 111.893 | 1.00 | 44.71 | 7 |
| ATOM | 1305 | CA | PHE | 210 | 96.457 | 255.954 | 112.268 | 1.00 | 49.18 | 6 |
| ATOM | 1306 | CB | PHE | 210 | 95.828 | 254.681 | 112.816 | 1.00 | 56.40 | 6 |
| ATOM | 1307 | CG | PHE | 210 | 95.223 | 254.854 | 114.177 | 1.00 | 60.12 | 6 |
| ATOM | 1308 | CD1 | PHE | 210 | 95.637 | 255.893 | 115.005 | 1.00 | 61.16 | 6 |
| ATOM | 1309 | CD2 | PHE | 210 | 94.230 | 254.003 | 114.622 | 1.00 | 55.90 | 6 |
| ATOM | 1310 | CE1 | PHE | 210 | 95.064 | 256.079 | 116.247 | 1.00 | 60.53 | 6 |
| ATOM | 1311 | CE2 | PHE | 210 | 93.660 | 254.187 | 115.857 | 1.00 | 58.70 | 6 |
| ATOM | 1312 | CZ | PHE | 210 | 94.077 | 255.226 | 116.670 | 1.00 | 58.39 | 6 |
| ATOM | 1313 | C | PHE | 210 | 97.422 | 255.642 | 111.138 | 1.00 | 50.47 | 6 |
| ATOM | 1314 | O | PHE | 210 | 98.411 | 254.951 | 111.349 | 1.00 | 50.23 | 8 |
| ATOM | 1315 | N | ALA | 211 | 97.140 | 256.194 | 109.958 | 1.00 | 51.05 | 7 |
| ATOM | 1316 | CA | ALA | 211 | 97.946 | 256.009 | 108.751 | 1.00 | 48.01 | 6 |
| ATOM | 1317 | CB | ALA | 211 | 97.361 | 256.800 | 107.609 | 1.00 | 47.95 | 6 |
| ATOM | 1318 | C | ALA | 211 | 99.385 | 256.403 | 108.918 | 1.00 | 50.58 | 6 |
| ATOM | 1319 | O | ALA | 211 | 99.687 | 257.390 | 109.586 | 1.00 | 50.34 | 8 |
| ATOM | 1320 | N | LYS | 212 | 100.265 | 255.627 | 108.291 | 1.00 | 53.63 | 7 |
| ATOM | 1321 | CA | LYS | 212 | 101.696 | 255.883 | 108.351 | 1.00 | 56.44 | 6 |
| ATOM | 1322 | CB | LYS | 212 | 102.353 | 255.148 | 109.539 | 1.00 | 63.40 | 6 |
| ATOM | 1323 | CG | LYS | 212 | 103.008 | 253.788 | 109.258 | 1.00 | 76.41 | 6 |
| ATOM | 1324 | CD | LYS | 212 | 104.079 | 253.462 | 110.333 | 1.00 | 82.06 | 6 |
| ATOM | 1325 | CE | LYS | 212 | 105.002 | 252.277 | 109.953 | 1.00 | 89.38 | 6 |
| ATOM | 1326 | NZ | LYS | 212 | 104.397 | 250.897 | 110.073 | 1.00 | 87.51 | 7 |
| ATOM | 1327 | C | LYS | 212 | 102.329 | 255.514 | 107.014 | 1.00 | 55.76 | 6 |
| ATOM | 1328 | O | LYS | 212 | 101.830 | 254.641 | 106.307 | 1.00 | 50.44 | 8 |
| ATOM | 1329 | N | GLU | 213 | 103.371 | 256.255 | 106.644 | 1.00 | 58.32 | 7 |
| ATOM | 1330 | CA | GLU | 213 | 104.100 | 256.047 | 105.398 | 1.00 | 67.18 | 6 |
| ATOM | 1331 | CB | GLU | 213 | 104.936 | 257.283 | 105.040 | 1.00 | 71.89 | 6 |
| ATOM | 1332 | CG | GLU | 213 | 104.204 | 258.611 | 105.074 | 1.00 | 86.72 | 6 |
| ATOM | 1333 | CD | GLU | 213 | 104.291 | 259.336 | 106.422 | 1.00 | 95.76 | 6 |
| ATOM | 1334 | OE1 | GLU | 213 | 104.964 | 260.393 | 106.477 | 1.00 | 97.93 | 8 |
| ATOM | 1335 | OE2 | GLU | 213 | 103.668 | 258.879 | 107.415 | 1.00 | 98.12 | 8 |
| ATOM | 1336 | C | GLU | 213 | 105.055 | 254.878 | 105.573 | 1.00 | 71.80 | 6 |
| ATOM | 1337 | O | GLU | 213 | 106.019 | 254.980 | 106.331 | 1.00 | 73.99 | 8 |
| ATOM | 1338 | N | THR | 214 | 104.798 | 253.781 | 104.867 | 1.00 | 75.36 | 7 |
| ATOM | 1339 | CA | THR | 214 | 105.640 | 252.593 | 104.948 | 1.00 | 77.86 | 6 |
| ATOM | 1340 | CB | THR | 214 | 104.870 | 251.329 | 104.538 | 1.00 | 76.30 | 6 |
| ATOM | 1341 | OG1 | THR | 214 | 104.327 | 251.499 | 103.226 | 1.00 | 75.42 | 8 |
| ATOM | 1342 | CG2 | THR | 214 | 103.743 | 251.070 | 105.498 | 1.00 | 79.20 | 6 |
| ATOM | 1343 | C | THR | 214 | 106.885 | 252.739 | 104.080 | 1.00 | 82.25 | 6 |
| ATOM | 1344 | O | THR | 214 | 107.031 | 252.089 | 103.042 | 1.00 | 83.65 | 8 |
| ATOM | 1345 | N | THR | 215 | 107.787 | 253.609 | 104.516 | 1.00 | 85.50 | 7 |
| ATOM | 1346 | CA | THR | 215 | 109.018 | 253.846 | 103.790 | 1.00 | 87.69 | 6 |
| ATOM | 1347 | CB | THR | 215 | 108.849 | 254.980 | 102.781 | 1.00 | 83.73 | 6 |
| ATOM | 1348 | OG1 | THR | 215 | 110.033 | 255.083 | 101.989 | 1.00 | 86.26 | 8 |
| ATOM | 1349 | CG2 | THR | 215 | 108.592 | 256.295 | 103.485 | 1.00 | 80.18 | 6 |
| ATOM | 1350 | C | THR | 215 | 110.163 | 254.165 | 104.747 | 1.00 | 92.77 | 6 |
| ATOM | 1351 | O | THR | 215 | 109.901 | 254.745 | 105.823 | 1.00 | 94.28 | 8 |
| ATOM | 1352 | OT | THR | 215 | 111.313 | 253.796 | 104.421 | 1.00 | 100.00 | 8 |
| ATOM | 1353 | CB | PRO | 227 | 89.937 | 244.648 | 120.244 | 1.00 | 83.11 | 6 |
| ATOM | 1354 | CG | PRO | 227 | 89.564 | 243.764 | 121.414 | 1.00 | 84.17 | 6 |
| ATOM | 1355 | C | PRO | 227 | 88.626 | 243.453 | 118.471 | 1.00 | 89.53 | 6 |
| ATOM | 1356 | O | PRO | 227 | 89.667 | 242.869 | 118.169 | 1.00 | 93.85 | 8 |
| ATOM | 1357 | N | PRO | 227 | 87.543 | 244.487 | 120.434 | 1.00 | 84.11 | 7 |
| ATOM | 1358 | CD | PRO | 227 | 88.101 | 244.125 | 121.750 | 1.00 | 81.36 | 6 |
| ATOM | 1359 | CA | PRO | 227 | 88.640 | 244.631 | 119.447 | 1.00 | 86.67 | 6 |
| ATOM | 1360 | N | TYR | 228 | 87.439 | 243.123 | 117.973 | 1.00 | 88.50 | 7 |
| ATOM | 1361 | CA | TYR | 228 | 87.257 | 242.020 | 117.034 | 1.00 | 87.60 | 6 |
| ATOM | 1362 | CB | TYR | 228 | 85.784 | 241.966 | 116.618 | 1.00 | 90.26 | 6 |
| ATOM | 1363 | CG | TYR | 228 | 84.819 | 241.893 | 117.784 | 1.00 | 98.08 | 6 |
| ATOM | 1364 | CD1 | TYR | 228 | 85.216 | 241.341 | 119.010 | 1.00 | 100.00 | 6 |
| ATOM | 1365 | CE1 | TYR | 228 | 84.322 | 241.211 | 120.085 | 1.00 | 100.00 | 6 |
| ATOM | 1366 | CD2 | TYR | 228 | 83.497 | 242.322 | 117.659 | 1.00 | 99.01 | 6 |
| ATOM | 1367 | CE2 | TYR | 228 | 82.586 | 242.191 | 118.732 | 1.00 | 100.00 | 6 |
| ATOM | 1368 | CZ | TYR | 228 | 83.011 | 241.630 | 119.943 | 1.00 | 100.00 | 6 |
| ATOM | 1369 | OH | TYR | 228 | 82.137 | 241.463 | 121.002 | 1.00 | 98.67 | 8 |
| ATOM | 1370 | C | TYR | 228 | 88.123 | 242.178 | 115.790 | 1.00 | 88.14 | 6 |
| ATOM | 1371 | O | TYR | 228 | 88.837 | 241.264 | 115.385 | 1.00 | 88.87 | 8 |
| ATOM | 1372 | N | TYR | 229 | 88.102 | 243.401 | 115.270 | 1.00 | 89.24 | 7 |
| ATOM | 1373 | CA | TYR | 229 | 88.814 | 243.854 | 114.077 | 1.00 | 85.42 | 6 |
| ATOM | 1374 | CB | TYR | 229 | 88.275 | 245.240 | 113.703 | 1.00 | 87.28 | 6 |
| ATOM | 1375 | CG | TYR | 229 | 86.817 | 245.341 | 114.070 | 1.00 | 94.81 | 6 |
| ATOM | 1376 | CD1 | TYR | 229 | 85.864 | 244.543 | 113.423 | 1.00 | 96.81 | 6 |
| ATOM | 1377 | CE1 | TYR | 229 | 84.555 | 244.425 | 113.909 | 1.00 | 95.27 | 6 |
| ATOM | 1378 | CD2 | TYR | 229 | 86.413 | 246.048 | 115.204 | 1.00 | 97.64 | 6 |
| ATOM | 1379 | CE2 | TYR | 229 | 85.100 | 245.934 | 115.696 | 1.00 | 99.46 | 6 |
| ATOM | 1380 | CZ | TYR | 229 | 84.186 | 245.114 | 115.046 | 1.00 | 95.00 | 6 |
| ATOM | 1381 | OH | TYR | 229 | 82.930 | 244.936 | 115.568 | 1.00 | 92.96 | 8 |
| ATOM | 1382 | C | TYR | 229 | 90.329 | 243.873 | 114.142 | 1.00 | 84.18 | 6 |
| ATOM | 1383 | O | TYR | 229 | 90.979 | 243.629 | 113.132 | 1.00 | 87.46 | 8 |
| ATOM | 1384 | N | VAL | 230 | 90.892 | 244.152 | 115.316 | 1.00 | 79.67 | 7 |
| ATOM | 1385 | CA | VAL | 230 | 92.345 | 244.199 | 115.483 | 1.00 | 74.92 | 6 |
| ATOM | 1386 | CB | VAL | 230 | 92.727 | 244.637 | 116.919 | 1.00 | 71.07 | 6 |
| ATOM | 1387 | CG1 | VAL | 230 | 94.220 | 244.579 | 117.120 | 1.00 | 73.09 | 6 |
| ATOM | 1388 | CG2 | VAL | 230 | 92.255 | 246.047 | 117.169 | 1.00 | 67.91 | 6 |
| ATOM | 1389 | C | VAL | 230 | 93.039 | 242.881 | 115.116 | 1.00 | 76.44 | 6 |
| ATOM | 1390 | O | VAL | 230 | 92.495 | 241.801 | 115.334 | 1.00 | 77.17 | 8 |
| ATOM | 1391 | N | ALA | 231 | 94.206 | 242.990 | 114.483 | 1.00 | 77.28 | 7 |
| ATOM | 1392 | CA | ALA | 231 | 94.989 | 241.830 | 114.069 | 1.00 | 76.81 | 6 |
| ATOM | 1393 | CB | ALA | 231 | 95.826 | 242.176 | 112.869 | 1.00 | 76.00 | 6 |
| ATOM | 1394 | C | ALA | 231 | 95.888 | 241.369 | 115.211 | 1.00 | 81.07 | 6 |
| ATOM | 1395 | O | ALA | 231 | 96.348 | 242.182 | 116.015 | 1.00 | 82.90 | 8 |
| ATOM | 1396 | N | PRO | 232 | 96.173 | 240.058 | 115.282 | 1.00 | 82.37 | 7 |
| ATOM | 1397 | CD | PRO | 232 | 95.707 | 239.014 | 114.359 | 1.00 | 81.62 | 6 |
| ATOM | 1398 | CA | PRO | 232 | 97.021 | 239.471 | 116.326 | 1.00 | 82.35 | 6 |
| ATOM | 1399 | CB | PRO | 232 | 97.041 | 237.986 | 115.958 | 1.00 | 81.66 | 6 |
| ATOM | 1400 | CG | PRO | 232 | 96.789 | 237.991 | 114.494 | 1.00 | 83.50 | 6 |
| ATOM | 1401 | C | PRO | 232 | 98.431 | 240.057 | 116.470 | 1.00 | 82.38 | 6 |
| ATOM | 1402 | O | PRO | 232 | 98.884 | 240.291 | 117.592 | 1.00 | 82.76 | 8 |
| ATOM | 1403 | N | GLU | 233 | 99.125 | 240.303 | 115.360 | 1.00 | 80.47 | 7 |
| ATOM | 1404 | CA | GLU | 233 | 100.471 | 240.863 | 115.455 | 1.00 | 82.16 | 6 |
| ATOM | 1405 | CB | GLU | 233 | 101.102 | 241.075 | 114.073 | 1.00 | 77.29 | 6 |
| ATOM | 1406 | CG | GLU | 233 | 100.446 | 242.129 | 113.197 | 1.00 | 68.78 | 6 |
| ATOM | 1407 | CD | GLU | 233 | 99.520 | 241.540 | 112.147 | 1.00 | 68.98 | 6 |
| ATOM | 1408 | OE1 | GLU | 233 | 99.340 | 242.181 | 111.089 | 1.00 | 68.64 | 8 |
| ATOM | 1409 | OE2 | GLU | 233 | 98.970 | 240.443 | 112.372 | 1.00 | 61.80 | 8 |
| ATOM | 1410 | C | GLU | 233 | 100.438 | 242.182 | 116.221 | 1.00 | 88.51 | 6 |
| ATOM | 1411 | O | GLU | 233 | 101.390 | 242.520 | 116.930 | 1.00 | 91.95 | 8 |
| ATOM | 1412 | N | VAL | 234 | 99.302 | 242.878 | 116.121 | 1.00 | 92.16 | 7 |
| ATOM | 1413 | CA | VAL | 234 | 99.079 | 244.167 | 116.781 | 1.00 | 92.42 | 6 |
| ATOM | 1414 | CB | VAL | 234 | 98.095 | 245.040 | 115.963 | 1.00 | 90.31 | 6 |
| ATOM | 1415 | CG1 | VAL | 234 | 98.124 | 246.490 | 116.444 | 1.00 | 88.48 | 6 |
| ATOM | 1416 | CG2 | VAL | 234 | 98.433 | 244.958 | 114.483 | 1.00 | 85.57 | 6 |
| ATOM | 1417 | C | VAL | 234 | 98.570 | 244.003 | 118.225 | 1.00 | 95.05 | 6 |
| ATOM | 1418 | O | VAL | 234 | 98.058 | 244.948 | 118.834 | 1.00 | 93.29 | 8 |
| ATOM | 1419 | N | LEU | 235 | 98.727 | 242.797 | 118.766 | 1.00 | 97.73 | 7 |
| ATOM | 1420 | CA | LEU | 235 | 98.300 | 242.495 | 120.129 | 1.00 | 99.51 | 6 |
| ATOM | 1421 | CB | LEU | 235 | 97.271 | 241.351 | 120.155 | 1.00 | 98.60 | 6 |
| ATOM | 1422 | CG | LEU | 235 | 95.825 | 241.652 | 119.732 | 1.00 | 95.25 | 6 |
| ATOM | 1423 | CD1 | LEU | 235 | 95.007 | 240.377 | 119.769 | 1.00 | 94.91 | 6 |
| ATOM | 1424 | CD2 | LEU | 235 | 95.203 | 242.686 | 120.645 | 1.00 | 91.60 | 6 |
| ATOM | 1425 | C | LEU | 235 | 99.489 | 242.171 | 121.040 | 1.00 | 100.00 | 6 |
| ATOM | 1426 | O | LEU | 235 | 99.302 | 241.684 | 122.159 | 1.00 | 100.00 | 8 |
| ATOM | 1427 | N | GLY | 236 | 100.708 | 242.413 | 120.554 | 1.00 | 99.29 | 7 |
| ATOM | 1428 | CA | GLY | 236 | 101.880 | 242.161 | 121.380 | 1.00 | 100.00 | 6 |
| ATOM | 1429 | C | GLY | 236 | 103.014 | 241.279 | 120.870 | 1.00 | 100.00 | 6 |
| ATOM | 1430 | O | GLY | 236 | 104.180 | 241.605 | 121.111 | 1.00 | 98.58 | 8 |
| ATOM | 1431 | N | PRO | 237 | 102.724 | 240.153 | 120.187 | 1.00 | 100.00 | 7 |
| ATOM | 1432 | CD | PRO | 237 | 101.389 | 239.630 | 119.835 | 1.00 | 100.00 | 6 |
| ATOM | 1433 | CA | PRO | 237 | 103.777 | 239.263 | 119.676 | 1.00 | 100.00 | 6 |
| ATOM | 1434 | CB | PRO | 237 | 102.999 | 238.332 | 118.745 | 1.00 | 100.00 | 6 |
| ATOM | 1435 | CG | PRO | 237 | 101.688 | 238.196 | 119.468 | 1.00 | 99.91 | 6 |
| ATOM | 1436 | C | PRO | 237 | 104.944 | 239.977 | 118.968 | 1.00 | 100.00 | 6 |
| ATOM | 1437 | O | PRO | 237 | 105.808 | 240.568 | 119.632 | 1.00 | 97.78 | 8 |
| ATOM | 1438 | N | GLU | 238 | 104.959 | 239.933 | 117.634 | 1.00 | 100.00 | 7 |
| ATOM | 1439 | CA | GLU | 238 | 106.018 | 240.576 | 116.853 | 1.00 | 100.00 | 6 |
| ATOM | 1440 | CB | GLU | 238 | 105.996 | 240.099 | 115.391 | 1.00 | 100.00 | 6 |
| ATOM | 1441 | CG | GLU | 238 | 105.447 | 238.690 | 115.165 | 1.00 | 100.00 | 6 |
| ATOM | 1442 | CD | GLU | 238 | 104.071 | 238.694 | 114.502 | 1.00 | 100.00 | 6 |
| ATOM | 1443 | OE1 | GLU | 238 | 103.963 | 239.203 | 113.359 | 1.00 | 100.00 | 8 |
| ATOM | 1444 | OE2 | GLU | 238 | 103.103 | 238.190 | 115.122 | 1.00 | 100.00 | 8 |
| ATOM | 1445 | C | GLU | 238 | 105.858 | 242.099 | 116.902 | 1.00 | 100.00 | 6 |
| ATOM | 1446 | O | GLU | 238 | 106.282 | 242.738 | 117.865 | 1.00 | 100.00 | 8 |
| ATOM | 1447 | N | LYS | 239 | 105.201 | 242.659 | 115.886 | 1.00 | 99.84 | 7 |
| ATOM | 1448 | CA | LYS | 239 | 104.971 | 244.098 | 115.790 | 1.00 | 100.00 | 6 |
| ATOM | 1449 | CB | LYS | 239 | 106.252 | 244.795 | 115.323 | 1.00 | 100.00 | 6 |
| ATOM | 1450 | CG | LYS | 239 | 106.650 | 246.005 | 116.158 | 1.00 | 100.00 | 6 |
| ATOM | 1451 | CD | LYS | 239 | 107.284 | 245.587 | 117.478 | 1.00 | 100.00 | 6 |
| ATOM | 1452 | CE | LYS | 239 | 108.548 | 244.762 | 117.253 | 1.00 | 100.00 | 6 |
| ATOM | 1453 | NZ | LYS | 239 | 109.077 | 244.229 | 118.539 | 1.00 | 100.00 | 7 |
| ATOM | 1454 | C | LYS | 239 | 103.845 | 244.365 | 114.783 | 1.00 | 100.00 | 6 |
| ATOM | 1455 | O | LYS | 239 | 102.912 | 243.567 | 114.665 | 1.00 | 100.00 | 8 |
| ATOM | 1456 | N | TYR | 240 | 103.907 | 245.513 | 114.103 | 1.00 | 100.00 | 7 |
| ATOM | 1457 | CA | TYR | 240 | 102.906 | 245.908 | 113.100 | 1.00 | 100.00 | 6 |
| ATOM | 1458 | CB | TYR | 240 | 101.645 | 246.516 | 113.752 | 1.00 | 100.00 | 6 |
| ATOM | 1459 | CG | TYR | 240 | 101.786 | 246.991 | 115.194 | 1.00 | 100.00 | 6 |
| ATOM | 1460 | CD1 | TYR | 240 | 101.896 | 248.351 | 115.496 | 1.00 | 100.00 | 6 |
| ATOM | 1461 | CE1 | TYR | 240 | 101.963 | 248.794 | 116.815 | 1.00 | 98.88 | 6 |
| ATOM | 1462 | CD2 | TYR | 240 | 101.752 | 246.082 | 116.260 | 1.00 | 100.00 | 6 |
| ATOM | 1463 | CE2 | TYR | 240 | 101.821 | 246.513 | 117.579 | 1.00 | 99.97 | 6 |
| ATOM | 1464 | CZ | TYR | 240 | 101.925 | 247.869 | 117.850 | 1.00 | 100.00 | 6 |
| ATOM | 1465 | OH | TYR | 240 | 101.996 | 248.302 | 119.156 | 1.00 | 100.00 | 8 |
| ATOM | 1466 | C | TYR | 240 | 103.520 | 246.883 | 112.081 | 1.00 | 100.00 | 6 |
| ATOM | 1467 | O | TYR | 240 | 104.577 | 247.474 | 112.353 | 1.00 | 100.00 | 8 |
| ATOM | 1468 | N | ASP | 241 | 102.895 | 247.020 | 110.902 | 1.00 | 100.00 | 7 |
| ATOM | 1469 | CA | ASP | 241 | 103.410 | 247.930 | 109.860 | 1.00 | 96.18 | 6 |
| ATOM | 1470 | CB | ASP | 241 | 104.752 | 247.422 | 109.269 | 1.00 | 100.00 | 6 |
| ATOM | 1471 | CG | ASP | 241 | 104.802 | 245.893 | 109.083 | 1.00 | 100.00 | 6 |
| ATOM | 1472 | OD1 | ASP | 241 | 103.789 | 245.282 | 108.675 | 1.00 | 100.00 | 8 |
| ATOM | 1473 | OD2 | ASP | 241 | 105.876 | 245.305 | 109.344 | 1.00 | 98.96 | 8 |
| ATOM | 1474 | C | ASP | 241 | 102.489 | 248.363 | 108.717 | 1.00 | 89.13 | 6 |
| ATOM | 1475 | O | ASP | 241 | 102.251 | 249.550 | 108.548 | 1.00 | 86.16 | 8 |
| ATOM | 1476 | N | LYS | 242 | 102.004 | 247.408 | 107.926 | 1.00 | 84.52 | 7 |
| ATOM | 1477 | CA | LYS | 242 | 101.120 | 247.690 | 106.789 | 1.00 | 81.66 | 6 |
| ATOM | 1478 | CB | LYS | 242 | 101.941 | 248.159 | 105.576 | 1.00 | 90.30 | 6 |
| ATOM | 1479 | CG | LYS | 242 | 102.807 | 247.066 | 104.915 | 1.00 | 99.46 | 6 |
| ATOM | 1480 | CD | LYS | 242 | 103.963 | 246.576 | 105.823 | 1.00 | 100.00 | 6 |
| ATOM | 1481 | CE | LYS | 242 | 104.456 | 245.158 | 105.462 | 1.00 | 100.00 | 6 |
| ATOM | 1482 | NZ | LYS | 242 | 105.261 | 245.082 | 104.200 | 1.00 | 99.42 | 7 |
| ATOM | 1483 | C | LYS | 242 | 100.359 | 246.444 | 106.371 | 1.00 | 75.64 | 6 |
| ATOM | 1484 | O | LYS | 242 | 99.879 | 246.341 | 105.242 | 1.00 | 66.08 | 8 |
| ATOM | 1485 | N | SER | 243 | 100.344 | 245.465 | 107.264 | 1.00 | 76.83 | 7 |
| ATOM | 1486 | CA | SER | 243 | 99.664 | 244.202 | 107.029 | 1.00 | 73.99 | 6 |
| ATOM | 1487 | CB | SER | 243 | 100.586 | 243.052 | 107.435 | 1.00 | 71.49 | 6 |
| ATOM | 1488 | OG | SER | 243 | 99.870 | 241.835 | 107.473 | 1.00 | 73.89 | 8 |
| ATOM | 1489 | C | SER | 243 | 98.340 | 244.130 | 107.801 | 1.00 | 72.34 | 6 |
| ATOM | 1490 | O | SER | 243 | 97.505 | 243.254 | 107.539 | 1.00 | 70.04 | 8 |
| ATOM | 1491 | N | CYS | 244 | 98.163 | 245.056 | 108.749 | 1.00 | 67.68 | 7 |
| ATOM | 1492 | CA | CYS | 244 | 96.958 | 245.120 | 109.566 | 1.00 | 61.25 | 6 |
| ATOM | 1493 | CB | CYS | 244 | 96.976 | 246.348 | 110.462 | 1.00 | 56.73 | 6 |
| ATOM | 1494 | SG | CYS | 244 | 98.539 | 246.728 | 111.218 | 1.00 | 73.70 | 16 |
| ATOM | 1495 | C | CYS | 244 | 95.768 | 245.253 | 108.645 | 1.00 | 60.20 | 6 |
| ATOM | 1496 | O | CYS | 244 | 94.699 | 244.708 | 108.917 | 1.00 | 64.77 | 8 |
| ATOM | 1497 | N | ASP | 245 | 95.966 | 245.991 | 107.556 | 1.00 | 54.68 | 7 |
| ATOM | 1498 | CA | ASP | 245 | 94.928 | 246.229 | 106.565 | 1.00 | 48.62 | 6 |
| ATOM | 1499 | CB | ASP | 245 | 95.462 | 247.137 | 105.449 | 1.00 | 45.94 | 6 |
| ATOM | 1500 | CG | ASP | 245 | 95.797 | 248.541 | 105.950 | 1.00 | 46.85 | 6 |
| ATOM | 1501 | OD1 | ASP | 245 | 95.365 | 248.885 | 107.074 | 1.00 | 54.27 | 8 |
| ATOM | 1502 | OD2 | ASP | 245 | 96.489 | 249.299 | 105.240 | 1.00 | 35.11 | 8 |
| ATOM | 1503 | C | ASP | 245 | 94.336 | 244.950 | 106.005 | 1.00 | 48.94 | 6 |
| ATOM | 1504 | O | ASP | 245 | 93.111 | 244.806 | 105.961 | 1.00 | 46.25 | 8 |
| ATOM | 1505 | N | MET | 246 | 95.201 | 243.994 | 105.666 | 1.00 | 50.39 | 7 |
| ATOM | 1506 | CA | MET | 246 | 94.755 | 242.718 | 105.115 | 1.00 | 51.29 | 6 |
| ATOM | 1507 | CB | MET | 246 | 95.926 | 241.912 | 104.572 | 1.00 | 47.87 | 6 |
| ATOM | 1508 | CG | MET | 246 | 96.678 | 242.633 | 103.479 | 1.00 | 52.16 | 6 |
| ATOM | 1509 | SD | MET | 246 | 95.621 | 243.393 | 102.238 | 1.00 | 49.79 | 16 |
| ATOM | 1510 | CE | MET | 246 | 94.887 | 241.958 | 101.484 | 1.00 | 52.46 | 6 |
| ATOM | 1511 | C | MET | 246 | 93.945 | 241.891 | 106.099 | 1.00 | 53.01 | 6 |
| ATOM | 1512 | O | MET | 246 | 93.013 | 241.191 | 105.694 | 1.00 | 53.41 | 8 |
| ATOM | 1513 | N | TRP | 247 | 94.305 | 241.950 | 107.381 | 1.00 | 54.63 | 7 |
| ATOM | 1514 | CA | TRP | 247 | 93.574 | 241.198 | 108.396 | 1.00 | 55.52 | 6 |
| ATOM | 1515 | CB | TRP | 247 | 94.240 | 241.318 | 109.774 | 1.00 | 56.45 | 6 |
| ATOM | 1516 | CG | TRP | 247 | 93.399 | 240.736 | 110.914 | 1.00 | 65.11 | 6 |
| ATOM | 1517 | CD2 | TRP | 247 | 93.396 | 239.377 | 111.381 | 1.00 | 68.69 | 6 |
| ATOM | 1518 | CE2 | TRP | 247 | 92.471 | 239.302 | 112.449 | 1.00 | 67.69 | 6 |
| ATOM | 1519 | CE3 | TRP | 247 | 94.090 | 238.216 | 111.006 | 1.00 | 67.51 | 6 |
| ATOM | 1520 | CD1 | TRP | 247 | 92.498 | 241.405 | 111.701 | 1.00 | 66.10 | 6 |
| ATOM | 1521 | NE1 | TRP | 247 | 91.941 | 240.553 | 112.623 | 1.00 | 64.36 | 7 |
| ATOM | 1522 | CZ2 | TRP | 247 | 92.223 | 238.110 | 113.144 | 1.00 | 66.74 | 6 |
| ATOM | 1523 | CZ3 | TRP | 247 | 93.843 | 237.034 | 111.701 | 1.00 | 64.67 | 6 |
| ATOM | 1524 | CH2 | TRP | 247 | 92.916 | 236.992 | 112.756 | 1.00 | 64.64 | 6 |
| ATOM | 1525 | C | TRP | 247 | 92.186 | 241.821 | 108.447 | 1.00 | 55.47 | 6 |
| ATOM | 1526 | O | TRP | 247 | 91.175 | 241.138 | 108.300 | 1.00 | 52.14 | 8 |
| ATOM | 1527 | N | SER | 248 | 92.165 | 243.142 | 108.578 | 1.00 | 59.79 | 7 |
| ATOM | 1528 | CA | SER | 248 | 90.926 | 243.893 | 108.647 | 1.00 | 63.08 | 6 |
| ATOM | 1529 | CB | SER | 248 | 91.217 | 245.390 | 108.646 | 1.00 | 65.65 | 6 |
| ATOM | 1530 | OG | SER | 248 | 91.911 | 245.763 | 109.829 | 1.00 | 70.45 | 8 |
| ATOM | 1531 | C | SER | 248 | 89.960 | 243.515 | 107.538 | 1.00 | 64.15 | 6 |
| ATOM | 1532 | O | SER | 248 | 88.764 | 243.336 | 107.789 | 1.00 | 63.30 | 8 |
| ATOM | 1533 | N | LEU | 249 | 90.486 | 243.343 | 106.326 | 1.00 | 65.08 | 7 |
| ATOM | 1534 | CA | LEU | 249 | 89.650 | 242.972 | 105.190 | 1.00 | 66.77 | 6 |
| ATOM | 1535 | CB | LEU | 249 | 90.477 | 242.853 | 103.909 | 1.00 | 67.67 | 6 |
| ATOM | 1536 | CG | LEU | 249 | 90.932 | 244.142 | 103.226 | 1.00 | 67.64 | 6 |
| ATOM | 1537 | CD1 | LEU | 249 | 91.941 | 243.850 | 102.104 | 1.00 | 62.51 | 6 |
| ATOM | 1538 | CD2 | LEU | 249 | 89.704 | 244.858 | 102.699 | 1.00 | 65.06 | 6 |
| ATOM | 1539 | C | LEU | 249 | 89.003 | 241.634 | 105.490 | 1.00 | 67.59 | 6 |
| ATOM | 1540 | O | LEU | 249 | 87.794 | 241.488 | 105.335 | 1.00 | 69.42 | 8 |
| ATOM | 1541 | N | GLY | 250 | 89.815 | 240.687 | 105.969 | 1.00 | 66.68 | 7 |
| ATOM | 1542 | CA | GLY | 250 | 89.341 | 239.346 | 106.299 | 1.00 | 64.64 | 6 |
| ATOM | 1543 | C | GLY | 250 | 88.247 | 239.308 | 107.353 | 1.00 | 62.65 | 6 |
| ATOM | 1544 | O | GLY | 250 | 87.255 | 238.589 | 107.211 | 1.00 | 62.59 | 8 |
| ATOM | 1545 | N | VAL | 251 | 88.446 | 240.069 | 108.425 | 1.00 | 58.91 | 7 |
| ATOM | 1546 | CA | VAL | 251 | 87.471 | 240.163 | 109.498 | 1.00 | 54.13 | 6 |
| ATOM | 1547 | CB | VAL | 251 | 87.979 | 241.105 | 110.567 | 1.00 | 44.55 | 6 |
| ATOM | 1548 | CG1 | VAL | 251 | 86.941 | 241.308 | 111.631 | 1.00 | 43.98 | 6 |
| ATOM | 1549 | CG2 | VAL | 251 | 89.252 | 240.578 | 111.133 | 1.00 | 40.69 | 6 |
| ATOM | 1550 | C | VAL | 251 | 86.176 | 240.744 | 108.928 | 1.00 | 59.21 | 6 |
| ATOM | 1551 | O | VAL | 251 | 85.081 | 240.252 | 109.200 | 1.00 | 61.29 | 8 |
| ATOM | 1552 | N | ILE | 252 | 86.327 | 241.776 | 108.104 | 1.00 | 60.74 | 7 |
| ATOM | 1553 | CA | ILE | 252 | 85.201 | 242.450 | 107.474 | 1.00 | 60.79 | 6 |
| ATOM | 1554 | CB | ILE | 252 | 85.667 | 243.794 | 106.824 | 1.00 | 58.77 | 6 |
| ATOM | 1555 | CG2 | ILE | 252 | 84.658 | 244.304 | 105.790 | 1.00 | 55.58 | 6 |
| ATOM | 1556 | CG1 | ILE | 252 | 85.879 | 244.835 | 107.925 | 1.00 | 52.79 | 6 |
| ATOM | 1557 | CD1 | ILE | 252 | 86.468 | 246.110 | 107.438 | 1.00 | 59.93 | 6 |
| ATOM | 1558 | C | ILE | 252 | 84.460 | 241.555 | 106.479 | 1.00 | 60.31 | 6 |
| ATOM | 1559 | O | ILE | 252 | 83.232 | 241.599 | 106.404 | 1.00 | 56.21 | 8 |
| ATOM | 1560 | N | MET | 253 | 85.203 | 240.714 | 105.762 | 1.00 | 61.63 | 7 |
| ATOM | 1561 | CA | MET | 253 | 84.610 | 239.810 | 104.783 | 1.00 | 65.41 | 6 |
| ATOM | 1562 | CB | MET | 253 | 85.695 | 239.152 | 103.920 | 1.00 | 63.78 | 6 |
| ATOM | 1563 | CG | MET | 253 | 85.148 | 238.512 | 102.652 | 1.00 | 65.10 | 6 |
| ATOM | 1564 | SD | MET | 253 | 86.307 | 237.496 | 101.749 | 1.00 | 70.31 | 16 |
| ATOM | 1565 | CE | MET | 253 | 87.046 | 238.636 | 100.685 | 1.00 | 64.35 | 6 |
| ATOM | 1566 | C | MET | 253 | 83.766 | 238.735 | 105.495 | 1.00 | 71.22 | 6 |
| ATOM | 1567 | O | MET | 253 | 82.744 | 238.287 | 104.962 | 1.00 | 74.37 | 8 |
| ATOM | 1568 | N | TYR | 254 | 84.189 | 238.322 | 106.694 | 1.00 | 69.39 | 7 |
| ATOM | 1569 | CA | TYR | 254 | 83.454 | 237.310 | 107.453 | 1.00 | 65.81 | 6 |
| ATOM | 1570 | CB | TYR | 254 | 84.239 | 236.895 | 108.699 | 1.00 | 65.59 | 6 |
| ATOM | 1571 | CG | TYR | 254 | 83.631 | 235.726 | 109.453 | 1.00 | 67.43 | 6 |
| ATOM | 1572 | CD1 | TYR | 254 | 82.676 | 235.933 | 110.442 | 1.00 | 67.39 | 6 |
| ATOM | 1573 | CE1 | TYR | 254 | 82.097 | 234.867 | 111.127 | 1.00 | 69.25 | 6 |
| ATOM | 1574 | CD2 | TYR | 254 | 84.002 | 234.410 | 109.166 | 1.00 | 67.21 | 6 |
| ATOM | 1575 | CE2 | TYR | 254 | 83.429 | 233.334 | 109.845 | 1.00 | 67.52 | 6 |
| ATOM | 1576 | CZ | TYR | 254 | 82.472 | 233.568 | 110.827 | 1.00 | 70.44 | 6 |
| ATOM | 1577 | OH | TYR | 254 | 81.872 | 232.515 | 111.497 | 1.00 | 64.64 | 8 |
| ATOM | 1578 | C | TYR | 254 | 82.088 | 237.884 | 107.862 | 1.00 | 66.99 | 6 |
| ATOM | 1579 | O | TYR | 254 | 81.046 | 237.300 | 107.563 | 1.00 | 68.68 | 8 |
| ATOM | 1580 | N | ILE | 255 | 82.105 | 239.057 | 108.497 | 1.00 | 65.08 | 7 |
| ATOM | 1581 | CA | ILE | 255 | 80.887 | 239.727 | 108.946 | 1.00 | 57.40 | 6 |
| ATOM | 1582 | CB | ILE | 255 | 81.188 | 241.111 | 109.593 | 1.00 | 53.71 | 6 |
| ATOM | 1583 | CG2 | ILE | 255 | 79.903 | 241.789 | 110.057 | 1.00 | 43.44 | 6 |
| ATOM | 1584 | CG1 | ILE | 255 | 82.125 | 240.948 | 110.791 | 1.00 | 49.82 | 6 |
| ATOM | 1585 | CD1 | ILE | 255 | 82.466 | 242.259 | 111.489 | 1.00 | 47.02 | 6 |
| ATOM | 1586 | C | ILE | 255 | 79.912 | 239.912 | 107.793 | 1.00 | 59.61 | 6 |
| ATOM | 1587 | O | ILE | 255 | 78.710 | 239.947 | 108.003 | 1.00 | 63.64 | 8 |
| ATOM | 1588 | N | LEU | 256 | 80.413 | 240.008 | 106.572 | 1.00 | 61.50 | 7 |
| ATOM | 1589 | CA | LEU | 256 | 79.506 | 240.184 | 105.450 | 1.00 | 71.57 | 6 |
| ATOM | 1590 | CB | LEU | 256 | 80.230 | 240.729 | 104.218 | 1.00 | 72.29 | 6 |
| ATOM | 1591 | CG | LEU | 256 | 80.267 | 242.243 | 104.010 | 1.00 | 70.71 | 6 |
| ATOM | 1592 | CD1 | LEU | 256 | 80.689 | 242.533 | 102.563 | 1.00 | 69.15 | 6 |
| ATOM | 1593 | CD2 | LEU | 256 | 78.910 | 242.844 | 104.308 | 1.00 | 61.55 | 6 |
| ATOM | 1594 | C | LEU | 256 | 78.741 | 238.924 | 105.061 | 1.00 | 76.51 | 6 |
| ATOM | 1595 | O | LEU | 256 | 77.509 | 238.948 | 104.965 | 1.00 | 81.67 | 8 |
| ATOM | 1596 | N | LEU | 257 | 79.468 | 237.829 | 104.857 | 1.00 | 76.11 | 7 |
| ATOM | 1597 | CA | LEU | 257 | 78.866 | 236.558 | 104.469 | 1.00 | 75.77 | 6 |
| ATOM | 1598 | CB | LEU | 257 | 79.948 | 235.588 | 103.999 | 1.00 | 74.28 | 6 |
| ATOM | 1599 | CG | LEU | 257 | 80.569 | 235.770 | 102.612 | 1.00 | 76.18 | 6 |
| ATOM | 1600 | CD1 | LEU | 257 | 79.530 | 235.506 | 101.539 | 1.00 | 80.60 | 6 |
| ATOM | 1601 | CD2 | LEU | 257 | 81.157 | 237.150 | 102.452 | 1.00 | 77.69 | 6 |
| ATOM | 1602 | C | LEU | 257 | 77.979 | 235.866 | 105.498 | 1.00 | 79.47 | 6 |
| ATOM | 1603 | O | LEU | 257 | 77.199 | 234.991 | 105.132 | 1.00 | 82.30 | 8 |
| ATOM | 1604 | N | CYS | 258 | 78.073 | 236.257 | 106.768 | 1.00 | 81.87 | 7 |
| ATOM | 1605 | CA | CYS | 258 | 77.254 | 235.634 | 107.808 | 1.00 | 84.11 | 6 |
| ATOM | 1606 | CB | CYS | 258 | 78.116 | 234.728 | 108.680 | 1.00 | 81.73 | 6 |
| ATOM | 1607 | SG | CYS | 258 | 79.297 | 235.619 | 109.695 | 1.00 | 78.97 | 16 |
| ATOM | 1608 | C | CYS | 258 | 76.486 | 236.612 | 108.699 | 1.00 | 87.63 | 6 |
| ATOM | 1609 | O | CYS | 258 | 75.325 | 236.364 | 109.026 | 1.00 | 93.56 | 8 |
| ATOM | 1610 | N | GLY | 259 | 77.148 | 237.681 | 109.144 | 1.00 | 88.18 | 7 |
| ATOM | 1611 | CA | GLY | 259 | 76.487 | 238.669 | 109.985 | 1.00 | 85.25 | 6 |
| ATOM | 1612 | C | GLY | 259 | 77.014 | 238.800 | 111.402 | 1.00 | 84.92 | 6 |
| ATOM | 1613 | O | GLY | 259 | 76.386 | 239.446 | 112.239 | 1.00 | 84.46 | 8 |
| ATOM | 1614 | N | TYR | 260 | 78.166 | 238.196 | 111.674 | 1.00 | 87.33 | 7 |
| ATOM | 1615 | CA | TYR | 260 | 78.784 | 238.244 | 113.003 | 1.00 | 90.48 | 6 |
| ATOM | 1616 | CB | TYR | 260 | 78.187 | 237.163 | 113.910 | 1.00 | 92.90 | 6 |
| ATOM | 1617 | CG | TYR | 260 | 78.151 | 235.784 | 113.283 | 1.00 | 94.92 | 6 |
| ATOM | 1618 | CD1 | TYR | 260 | 77.125 | 235.429 | 112.409 | 1.00 | 93.88 | 6 |
| ATOM | 1619 | CE1 | TYR | 260 | 77.082 | 234.181 | 111.826 | 1.00 | 94.27 | 6 |
| ATOM | 1620 | CD2 | TYR | 260 | 79.143 | 234.839 | 113.558 | 1.00 | 93.73 | 6 |
| ATOM | 1621 | CE2 | TYR | 260 | 79.109 | 233.584 | 112.976 | 1.00 | 93.88 | 6 |
| ATOM | 1622 | CZ | TYR | 260 | 78.072 | 233.264 | 112.111 | 1.00 | 95.62 | 6 |
| ATOM | 1623 | OH | TYR | 260 | 78.011 | 232.024 | 111.526 | 1.00 | 100.00 | 8 |
| ATOM | 1624 | C | TYR | 260 | 80.292 | 238.046 | 112.890 | 1.00 | 91.76 | 6 |
| ATOM | 1625 | O | TYR | 260 | 80.762 | 237.263 | 112.074 | 1.00 | 94.22 | 8 |
| ATOM | 1626 | N | PRO | 261 | 81.068 | 238.729 | 113.736 | 1.00 | 92.32 | 7 |
| ATOM | 1627 | CD | PRO | 261 | 80.602 | 239.652 | 114.780 | 1.00 | 94.11 | 6 |
| ATOM | 1628 | CA | PRO | 261 | 82.534 | 238.636 | 113.733 | 1.00 | 95.28 | 6 |
| ATOM | 1629 | CB | PRO | 261 | 82.930 | 239.496 | 114.934 | 1.00 | 95.01 | 6 |
| ATOM | 1630 | CG | PRO | 261 | 81.826 | 240.495 | 115.014 | 1.00 | 97.25 | 6 |
| ATOM | 1631 | C | PRO | 261 | 83.059 | 237.214 | 113.903 | 1.00 | 96.16 | 6 |
| ATOM | 1632 | O | PRO | 261 | 82.473 | 236.424 | 114.638 | 1.00 | 98.72 | 8 |
| ATOM | 1633 | N | PRO | 262 | 84.160 | 236.865 | 113.205 | 1.00 | 95.84 | 7 |
| ATOM | 1634 | CD | PRO | 262 | 84.965 | 237.679 | 112.278 | 1.00 | 97.74 | 6 |
| ATOM | 1635 | CA | PRO | 262 | 84.724 | 235.520 | 113.330 | 1.00 | 94.34 | 6 |
| ATOM | 1636 | CB | PRO | 262 | 85.857 | 235.526 | 112.298 | 1.00 | 93.04 | 6 |
| ATOM | 1637 | CG | PRO | 262 | 86.285 | 236.942 | 112.266 | 1.00 | 93.98 | 6 |
| ATOM | 1638 | C | PRO | 262 | 85.228 | 235.341 | 114.763 | 1.00 | 94.02 | 6 |
| ATOM | 1639 | O | PRO | 262 | 85.263 | 234.231 | 115.283 | 1.00 | 98.20 | 8 |
| ATOM | 1640 | N | PHE | 263 | 85.600 | 236.452 | 115.391 | 1.00 | 90.37 | 7 |
| ATOM | 1641 | CA | PHE | 263 | 86.091 | 236.463 | 116.760 | 1.00 | 90.96 | 6 |
| ATOM | 1642 | CB | PHE | 263 | 87.547 | 236.919 | 116.828 | 1.00 | 87.79 | 6 |
| ATOM | 1643 | CG | PHE | 263 | 88.481 | 236.099 | 115.984 | 1.00 | 88.65 | 6 |
| ATOM | 1644 | CD1 | PHE | 263 | 89.037 | 234.925 | 116.479 | 1.00 | 87.25 | 6 |
| ATOM | 1645 | CD2 | PHE | 263 | 88.798 | 236.494 | 114.683 | 1.00 | 88.06 | 6 |
| ATOM | 1646 | CE1 | PHE | 263 | 89.893 | 234.156 | 115.690 | 1.00 | 85.42 | 6 |
| ATOM | 1647 | CE2 | PHE | 263 | 89.653 | 235.731 | 113.890 | 1.00 | 84.34 | 6 |
| ATOM | 1648 | CZ | PHE | 263 | 90.199 | 234.561 | 114.396 | 1.00 | 83.14 | 6 |
| ATOM | 1649 | C | PHE | 263 | 85.207 | 237.514 | 117.395 | 1.00 | 95.73 | 6 |
| ATOM | 1650 | O | PHE | 263 | 84.999 | 238.574 | 116.800 | 1.00 | 98.83 | 8 |
| ATOM | 1651 | N | TYR | 264 | 84.662 | 237.214 | 118.573 | 1.00 | 99.85 | 7 |
| ATOM | 1652 | CA | TYR | 264 | 83.781 | 238.149 | 119.288 | 1.00 | 100.00 | 6 |
| ATOM | 1653 | CB | TYR | 264 | 82.338 | 238.028 | 118.771 | 1.00 | 98.26 | 6 |
| ATOM | 1654 | CG | TYR | 264 | 81.836 | 236.608 | 118.589 | 1.00 | 96.85 | 6 |
| ATOM | 1655 | CD1 | TYR | 264 | 82.506 | 235.521 | 119.159 | 1.00 | 93.27 | 6 |
| ATOM | 1656 | CE1 | TYR | 264 | 82.067 | 234.224 | 118.967 | 1.00 | 95.74 | 6 |
| ATOM | 1657 | CD2 | TYR | 264 | 80.702 | 236.353 | 117.820 | 1.00 | 96.01 | 6 |
| ATOM | 1658 | CE2 | TYR | 264 | 80.251 | 235.059 | 117.620 | 1.00 | 99.17 | 6 |
| ATOM | 1659 | CZ | TYR | 264 | 80.938 | 233.997 | 118.194 | 1.00 | 100.00 | 6 |
| ATOM | 1660 | OH | TYR | 264 | 80.503 | 232.708 | 117.977 | 1.00 | 100.00 | 8 |
| ATOM | 1661 | C | TYR | 264 | 83.838 | 238.034 | 120.821 | 1.00 | 100.00 | 6 |
| ATOM | 1662 | O | TYR | 264 | 84.925 | 238.168 | 121.404 | 1.00 | 100.00 | 8 |
| ATOM | 1663 | N | SER | 265 | 82.674 | 237.846 | 121.460 | 1.00 | 100.00 | 7 |
| ATOM | 1664 | CA | SER | 265 | 82.544 | 237.713 | 122.922 | 1.00 | 100.00 | 6 |
| ATOM | 1665 | CB | SER | 265 | 83.525 | 236.653 | 123.461 | 1.00 | 100.00 | 6 |
| ATOM | 1666 | OG | SER | 265 | 83.478 | 236.552 | 124.877 | 1.00 | 100.00 | 8 |
| ATOM | 1667 | C | SER | 265 | 82.675 | 239.017 | 123.728 | 1.00 | 100.00 | 6 |
| ATOM | 1668 | O | SER | 265 | 83.354 | 239.963 | 123.310 | 1.00 | 100.00 | 8 |
| ATOM | 1669 | N | ASN | 266 | 82.013 | 239.054 | 124.887 | 1.00 | 100.00 | 7 |
| ATOM | 1670 | CA | ASN | 266 | 82.045 | 240.230 | 125.765 | 1.00 | 100.00 | 6 |
| ATOM | 1671 | CB | ASN | 266 | 80.951 | 240.161 | 126.866 | 1.00 | 100.00 | 6 |
| ATOM | 1672 | CG | ASN | 266 | 80.983 | 238.859 | 127.692 | 1.00 | 100.00 | 6 |
| ATOM | 1673 | OD1 | ASN | 266 | 80.179 | 237.944 | 127.465 | 1.00 | 100.00 | 8 |
| ATOM | 1674 | ND2 | ASN | 266 | 81.874 | 238.801 | 128.687 | 1.00 | 100.00 | 7 |
| ATOM | 1675 | C | ASN | 266 | 83.434 | 240.451 | 126.368 | 1.00 | 100.00 | 6 |
| ATOM | 1676 | O | ASN | 266 | 83.803 | 241.582 | 126.706 | 1.00 | 99.50 | 8 |
| ATOM | 1677 | N | HIS | 267 | 84.200 | 239.361 | 126.468 | 1.00 | 100.00 | 7 |
| ATOM | 1678 | CA | HIS | 267 | 85.558 | 239.382 | 127.016 | 1.00 | 100.00 | 6 |
| ATOM | 1679 | CB | HIS | 267 | 85.548 | 239.702 | 128.517 | 1.00 | 99.65 | 6 |
| ATOM | 1680 | CG | HIS | 267 | 86.670 | 240.596 | 128.947 | 1.00 | 100.00 | 6 |
| ATOM | 1681 | CD2 | HIS | 267 | 87.569 | 241.309 | 128.225 | 1.00 | 100.00 | 6 |
| ATOM | 1682 | ND1 | HIS | 267 | 86.946 | 240.861 | 130.271 | 1.00 | 100.00 | 7 |
| ATOM | 1683 | CE1 | HIS | 267 | 87.965 | 241.700 | 130.348 | 1.00 | 100.00 | 6 |
| ATOM | 1684 | NE2 | HIS | 267 | 88.361 | 241.986 | 129.121 | 1.00 | 100.00 | 7 |
| ATOM | 1685 | C | HIS | 267 | 86.284 | 238.057 | 126.779 | 1.00 | 100.00 | 6 |
| ATOM | 1686 | O | HIS | 267 | 85.609 | 237.001 | 126.781 | 1.00 | 99.13 | 8 |
| ATOM | 1687 | OT | HIS | 267 | 87.522 | 238.099 | 126.591 | 1.00 | 100.00 | 8 |
| ATOM | 1688 | CB | ALA | 275 | 87.847 | 237.517 | 122.968 | 1.00 | 71.75 | 6 |
| ATOM | 1689 | C | ALA | 275 | 90.121 | 236.696 | 122.240 | 1.00 | 77.78 | 6 |
| ATOM | 1690 | O | ALA | 275 | 90.486 | 236.200 | 121.171 | 1.00 | 74.24 | 8 |
| ATOM | 1691 | N | ALA | 275 | 88.962 | 235.648 | 124.194 | 1.00 | 72.73 | 7 |
| ATOM | 1692 | CA | ALA | 275 | 88.778 | 236.298 | 122.860 | 1.00 | 76.10 | 6 |
| ATOM | 1693 | N | ALA | 276 | 90.863 | 237.571 | 122.921 | 1.00 | 80.81 | 7 |
| ATOM | 1694 | CA | ALA | 276 | 92.163 | 238.023 | 122.426 | 1.00 | 84.26 | 6 |
| ATOM | 1695 | CB | ALA | 276 | 92.769 | 239.068 | 123.370 | 1.00 | 79.85 | 6 |
| ATOM | 1696 | C | ALA | 276 | 93.089 | 236.815 | 122.283 | 1.00 | 89.16 | 6 |
| ATOM | 1697 | O | ALA | 276 | 94.123 | 236.885 | 121.607 | 1.00 | 92.66 | 8 |
| ATOM | 1698 | N | ALA | 277 | 92.708 | 235.717 | 122.943 | 1.00 | 92.55 | 7 |
| ATOM | 1699 | CA | ALA | 277 | 93.462 | 234.462 | 122.917 | 1.00 | 91.98 | 6 |
| ATOM | 1700 | CB | ALA | 277 | 93.171 | 233.632 | 124.179 | 1.00 | 86.98 | 6 |
| ATOM | 1701 | C | ALA | 277 | 93.016 | 233.710 | 121.661 | 1.00 | 89.40 | 6 |
| ATOM | 1702 | O | ALA | 277 | 93.836 | 233.378 | 120.796 | 1.00 | 87.90 | 8 |
| ATOM | 1703 | N | ALA | 278 | 91.700 | 233.525 | 121.538 | 1.00 | 84.60 | 7 |
| ATOM | 1704 | CA | ALA | 278 | 91.109 | 232.831 | 120.401 | 1.00 | 81.23 | 6 |
| ATOM | 1705 | CB | ALA | 278 | 89.592 | 232.818 | 120.519 | 1.00 | 80.32 | 6 |
| ATOM | 1706 | C | ALA | 278 | 91.547 | 233.496 | 119.102 | 1.00 | 81.59 | 6 |
| ATOM | 1707 | O | ALA | 278 | 91.612 | 232.847 | 118.062 | 1.00 | 81.48 | 8 |
| ATOM | 1708 | N | ILE | 279 | 91.851 | 234.791 | 119.172 | 1.00 | 81.36 | 7 |
| ATOM | 1709 | CA | ILE | 279 | 92.293 | 235.536 | 118.004 | 1.00 | 80.80 | 6 |
| ATOM | 1710 | CB | ILE | 279 | 92.277 | 237.069 | 118.233 | 1.00 | 81.66 | 6 |
| ATOM | 1711 | CG2 | ILE | 279 | 93.338 | 237.759 | 117.375 | 1.00 | 78.92 | 6 |
| ATOM | 1712 | CG1 | ILE | 279 | 90.898 | 237.638 | 117.895 | 1.00 | 84.39 | 6 |
| ATOM | 1713 | CD1 | ILE | 279 | 90.782 | 239.156 | 118.077 | 1.00 | 81.98 | 6 |
| ATOM | 1714 | C | ILE | 279 | 93.707 | 235.111 | 117.680 | 1.00 | 80.72 | 6 |
| ATOM | 1715 | O | ILE | 279 | 93.976 | 234.681 | 116.565 | 1.00 | 82.68 | 8 |
| ATOM | 1716 | N | ARG | 280 | 94.605 | 235.202 | 118.657 | 1.00 | 81.91 | 7 |
| ATOM | 1717 | CA | ARG | 280 | 95.994 | 234.817 | 118.422 | 1.00 | 86.81 | 6 |
| ATOM | 1718 | CB | ARG | 280 | 96.904 | 235.269 | 119.564 | 1.00 | 87.27 | 6 |
| ATOM | 1719 | CG | ARG | 280 | 97.051 | 236.786 | 119.620 | 1.00 | 91.90 | 6 |
| ATOM | 1720 | CD | ARG | 280 | 98.335 | 237.223 | 120.307 | 1.00 | 96.88 | 6 |
| ATOM | 1721 | NE | ARG | 280 | 98.447 | 236.689 | 121.665 | 1.00 | 100.00 | 7 |
| ATOM | 1722 | CZ | ARG | 280 | 99.076 | 237.298 | 122.668 | 1.00 | 100.00 | 6 |
| ATOM | 1723 | NH1 | ARG | 280 | 99.658 | 238.480 | 122.475 | 1.00 | 100.00 | 7 |
| ATOM | 1724 | NH2 | ARG | 280 | 99.128 | 236.718 | 123.866 | 1.00 | 99.94 | 7 |
| ATOM | 1725 | C | ARG | 280 | 96.197 | 233.346 | 118.072 | 1.00 | 87.87 | 6 |
| ATOM | 1726 | O | ARG | 280 | 97.208 | 232.975 | 117.470 | 1.00 | 87.49 | 8 |
| ATOM | 1727 | N | MET | 281 | 95.219 | 232.518 | 118.418 | 1.00 | 88.64 | 7 |
| ATOM | 1728 | CA | MET | 281 | 95.294 | 231.091 | 118.125 | 1.00 | 89.59 | 6 |
| ATOM | 1729 | CB | MET | 281 | 94.341 | 230.299 | 119.024 | 1.00 | 88.02 | 6 |
| ATOM | 1730 | CG | MET | 281 | 94.790 | 230.135 | 120.466 | 1.00 | 85.68 | 6 |
| ATOM | 1731 | SD | MET | 281 | 96.047 | 228.875 | 120.668 | 1.00 | 81.12 | 16 |
| ATOM | 1732 | CE | MET | 281 | 95.053 | 227.403 | 120.638 | 1.00 | 82.63 | 6 |
| ATOM | 1733 | C | MET | 281 | 94.855 | 230.912 | 116.678 | 1.00 | 90.40 | 6 |
| ATOM | 1734 | O | MET | 281 | 95.266 | 229.963 | 116.011 | 1.00 | 94.24 | 8 |
| ATOM | 1735 | N | GLY | 282 | 94.051 | 231.860 | 116.196 | 1.00 | 88.92 | 7 |
| ATOM | 1736 | CA | GLY | 282 | 93.523 | 231.796 | 114.845 | 1.00 | 85.88 | 6 |
| ATOM | 1737 | C | GLY | 282 | 92.422 | 230.760 | 114.886 | 1.00 | 86.15 | 6 |
| ATOM | 1738 | O | GLY | 282 | 92.349 | 229.871 | 114.032 | 1.00 | 85.30 | 8 |
| ATOM | 1739 | N | GLN | 283 | 91.581 | 230.872 | 115.915 | 1.00 | 84.85 | 7 |
| ATOM | 1740 | CA | GLN | 283 | 90.482 | 229.945 | 116.151 | 1.00 | 83.57 | 6 |
| ATOM | 1741 | CB | GLN | 283 | 90.465 | 229.552 | 117.630 | 1.00 | 84.62 | 6 |
| ATOM | 1742 | CG | GLN | 283 | 89.545 | 228.389 | 117.962 | 1.00 | 87.92 | 6 |
| ATOM | 1743 | CD | GLN | 283 | 89.383 | 228.185 | 119.457 | 1.00 | 89.75 | 6 |
| ATOM | 1744 | OE1 | GLN | 283 | 90.294 | 228.477 | 120.249 | 1.00 | 83.71 | 8 |
| ATOM | 1745 | NE2 | GLN | 283 | 88.218 | 227.678 | 119.855 | 1.00 | 88.55 | 7 |
| ATOM | 1746 | C | GLN | 283 | 89.114 | 230.478 | 115.737 | 1.00 | 83.15 | 6 |
| ATOM | 1747 | O | GLN | 283 | 88.310 | 230.886 | 116.579 | 1.00 | 83.14 | 8 |
| ATOM | 1748 | N | TYR | 284 | 88.852 | 230.469 | 114.435 | 1.00 | 84.76 | 7 |
| ATOM | 1749 | CA | TYR | 284 | 87.576 | 230.949 | 113.903 | 1.00 | 84.23 | 6 |
| ATOM | 1750 | CB | TYR | 284 | 87.782 | 232.158 | 112.965 | 1.00 | 77.45 | 6 |
| ATOM | 1751 | CG | TYR | 284 | 88.813 | 231.955 | 111.875 | 1.00 | 64.97 | 6 |
| ATOM | 1752 | CD1 | TYR | 284 | 88.435 | 231.520 | 110.615 | 1.00 | 64.14 | 6 |
| ATOM | 1753 | CE1 | TYR | 284 | 89.377 | 231.309 | 109.608 | 1.00 | 71.84 | 6 |
| ATOM | 1754 | CD2 | TYR | 284 | 90.167 | 232.186 | 112.114 | 1.00 | 66.79 | 6 |
| ATOM | 1755 | CE2 | TYR | 284 | 91.126 | 231.984 | 111.121 | 1.00 | 70.67 | 6 |
| ATOM | 1756 | CZ | TYR | 284 | 90.723 | 231.540 | 109.866 | 1.00 | 75.84 | 6 |
| ATOM | 1757 | OH | TYR | 284 | 91.653 | 231.302 | 108.873 | 1.00 | 74.32 | 8 |
| ATOM | 1758 | C | TYR | 284 | 86.864 | 229.814 | 113.178 | 1.00 | 85.85 | 6 |
| ATOM | 1759 | O | TYR | 284 | 87.469 | 229.090 | 112.383 | 1.00 | 87.41 | 8 |
| ATOM | 1760 | N | GLU | 285 | 85.583 | 229.641 | 113.471 | 1.00 | 85.92 | 7 |
| ATOM | 1761 | CA | GLU | 285 | 84.816 | 228.582 | 112.832 | 1.00 | 88.53 | 6 |
| ATOM | 1762 | CB | GLU | 285 | 83.998 | 227.815 | 113.882 | 1.00 | 91.78 | 6 |
| ATOM | 1763 | CG | GLU | 285 | 84.808 | 227.351 | 115.102 | 1.00 | 97.83 | 6 |
| ATOM | 1764 | CD | GLU | 285 | 86.105 | 226.625 | 114.733 | 1.00 | 100.00 | 6 |
| ATOM | 1765 | OE1 | GLU | 285 | 87.197 | 227.168 | 115.020 | 1.00 | 100.00 | 8 |
| ATOM | 1766 | OE2 | GLU | 285 | 86.036 | 225.511 | 114.164 | 1.00 | 100.00 | 8 |
| ATOM | 1767 | C | GLU | 285 | 83.889 | 229.132 | 111.763 | 1.00 | 86.91 | 6 |
| ATOM | 1768 | O | GLU | 285 | 83.602 | 230.323 | 111.751 | 1.00 | 88.23 | 8 |
| ATOM | 1769 | N | PHE | 286 | 83.455 | 228.264 | 110.851 | 1.00 | 86.96 | 7 |
| ATOM | 1770 | CA | PHE | 286 | 82.546 | 228.641 | 109.764 | 1.00 | 90.35 | 6 |
| ATOM | 1771 | CB | PHE | 286 | 83.085 | 228.171 | 108.395 | 1.00 | 87.31 | 6 |
| ATOM | 1772 | CG | PHE | 286 | 84.132 | 229.068 | 107.783 | 1.00 | 81.90 | 6 |
| ATOM | 1773 | CD1 | PHE | 286 | 84.140 | 230.441 | 108.022 | 1.00 | 84.48 | 6 |
| ATOM | 1774 | CD2 | PHE | 286 | 85.111 | 228.531 | 106.953 | 1.00 | 79.50 | 6 |
| ATOM | 1775 | CE1 | PHE | 286 | 85.110 | 231.267 | 107.447 | 1.00 | 81.25 | 6 |
| ATOM | 1776 | CE2 | PHE | 286 | 86.086 | 229.342 | 106.371 | 1.00 | 80.35 | 6 |
| ATOM | 1777 | CZ | PHE | 286 | 86.088 | 230.714 | 106.618 | 1.00 | 81.62 | 6 |
| ATOM | 1778 | C | PHE | 286 | 81.195 | 227.949 | 110.024 | 1.00 | 93.74 | 6 |
| ATOM | 1779 | O | PHE | 286 | 80.821 | 227.020 | 109.300 | 1.00 | 97.91 | 8 |
| ATOM | 1780 | N | PRO | 287 | 80.424 | 228.417 | 111.028 | 1.00 | 96.58 | 7 |
| ATOM | 1781 | CD | PRO | 287 | 80.646 | 229.619 | 111.848 | 1.00 | 96.93 | 6 |
| ATOM | 1782 | CA | PRO | 287 | 79.124 | 227.817 | 111.359 | 1.00 | 98.72 | 6 |
| ATOM | 1783 | CB | PRO | 287 | 78.481 | 228.879 | 112.248 | 1.00 | 96.06 | 6 |
| ATOM | 1784 | CG | PRO | 287 | 79.650 | 229.426 | 112.971 | 1.00 | 96.25 | 6 |
| ATOM | 1785 | C | PRO | 287 | 78.246 | 227.488 | 110.157 | 1.00 | 100.00 | 6 |
| ATOM | 1786 | O | PRO | 287 | 77.943 | 228.365 | 109.353 | 1.00 | 100.00 | 8 |
| ATOM | 1787 | N | ASN | 288 | 77.837 | 226.219 | 110.063 | 1.00 | 100.00 | 7 |
| ATOM | 1788 | CA | ASN | 288 | 76.988 | 225.726 | 108.972 | 1.00 | 99.97 | 6 |
| ATOM | 1789 | CB | ASN | 288 | 76.934 | 224.192 | 108.978 | 1.00 | 100.00 | 6 |
| ATOM | 1790 | CG | ASN | 288 | 78.240 | 223.554 | 108.494 | 1.00 | 100.00 | 6 |
| ATOM | 1791 | OD1 | ASN | 288 | 78.594 | 222.442 | 108.907 | 1.00 | 100.00 | 8 |
| ATOM | 1792 | ND2 | ASN | 288 | 78.953 | 224.251 | 107.604 | 1.00 | 100.00 | 7 |
| ATOM | 1793 | C | ASN | 288 | 75.572 | 226.313 | 108.887 | 1.00 | 99.86 | 6 |
| ATOM | 1794 | O | ASN | 288 | 75.004 | 226.395 | 107.797 | 1.00 | 98.15 | 8 |
| ATOM | 1795 | N | PRO | 289 | 74.964 | 226.695 | 110.030 | 1.00 | 100.00 | 7 |
| ATOM | 1796 | CD | PRO | 289 | 75.342 | 226.519 | 111.443 | 1.00 | 99.94 | 6 |
| ATOM | 1797 | CA | PRO | 289 | 73.616 | 227.266 | 109.932 | 1.00 | 99.25 | 6 |
| ATOM | 1798 | CB | PRO | 289 | 73.237 | 227.506 | 111.393 | 1.00 | 99.05 | 6 |
| ATOM | 1799 | CG | PRO | 289 | 74.571 | 227.618 | 112.093 | 1.00 | 100.00 | 6 |
| ATOM | 1800 | C | PRO | 289 | 73.581 | 228.562 | 109.121 | 1.00 | 98.81 | 6 |
| ATOM | 1801 | O | PRO | 289 | 72.509 | 229.030 | 108.748 | 1.00 | 99.96 | 8 |
| ATOM | 1802 | N | GLU | 290 | 74.756 | 229.131 | 108.849 | 1.00 | 98.86 | 7 |
| ATOM | 1803 | CA | GLU | 290 | 74.868 | 230.372 | 108.078 | 1.00 | 98.50 | 6 |
| ATOM | 1804 | CB | GLU | 290 | 75.124 | 231.577 | 109.003 | 1.00 | 100.00 | 6 |
| ATOM | 1805 | CG | GLU | 290 | 73.878 | 232.222 | 109.634 | 1.00 | 100.00 | 6 |
| ATOM | 1806 | CD | GLU | 290 | 73.688 | 231.868 | 111.103 | 1.00 | 100.00 | 6 |
| ATOM | 1807 | OE1 | GLU | 290 | 74.690 | 231.820 | 111.852 | 1.00 | 100.00 | 8 |
| ATOM | 1808 | OE2 | GLU | 290 | 72.528 | 231.652 | 111.511 | 1.00 | 98.65 | 8 |
| ATOM | 1809 | C | GLU | 290 | 75.972 | 230.322 | 107.019 | 1.00 | 95.42 | 6 |
| ATOM | 1810 | O | GLU | 290 | 75.966 | 231.112 | 106.085 | 1.00 | 93.17 | 8 |
| ATOM | 1811 | N | TRP | 291 | 76.892 | 229.372 | 107.154 | 1.00 | 97.25 | 7 |
| ATOM | 1812 | CA | TRP | 291 | 78.010 | 229.213 | 106.221 | 1.00 | 98.31 | 6 |
| ATOM | 1813 | CB | TRP | 291 | 79.327 | 229.167 | 106.991 | 1.00 | 99.73 | 6 |
| ATOM | 1814 | CG | TRP | 291 | 79.835 | 230.505 | 107.415 | 1.00 | 99.76 | 6 |
| ATOM | 1815 | CD2 | TRP | 291 | 80.652 | 231.389 | 106.640 | 1.00 | 97.41 | 6 |
| ATOM | 1816 | CE2 | TRP | 291 | 80.930 | 232.507 | 107.444 | 1.00 | 94.32 | 6 |
| ATOM | 1817 | CE3 | TRP | 291 | 81.185 | 231.333 | 105.348 | 1.00 | 98.04 | 6 |
| ATOM | 1818 | CD1 | TRP | 291 | 79.648 | 231.111 | 108.618 | 1.00 | 97.37 | 6 |
| ATOM | 1819 | NE1 | TRP | 291 | 80.304 | 232.313 | 108.644 | 1.00 | 96.11 | 7 |
| ATOM | 1820 | CZ2 | TRP | 291 | 81.709 | 233.566 | 106.999 | 1.00 | 96.95 | 6 |
| ATOM | 1821 | CZ3 | TRP | 291 | 81.960 | 232.383 | 104.907 | 1.00 | 98.79 | 6 |
| ATOM | 1822 | CH2 | TRP | 291 | 82.218 | 233.486 | 105.733 | 1.00 | 99.72 | 6 |
| ATOM | 1823 | C | TRP | 291 | 77.965 | 228.001 | 105.304 | 1.00 | 99.56 | 6 |
| ATOM | 1824 | O | TRP | 291 | 78.707 | 227.946 | 104.319 | 1.00 | 99.23 | 8 |
| ATOM | 1825 | N | SER | 292 | 77.129 | 227.023 | 105.649 | 1.00 | 100.00 | 7 |
| ATOM | 1826 | CA | SER | 292 | 76.979 | 225.792 | 104.867 | 1.00 | 100.00 | 6 |
| ATOM | 1827 | CB | SER | 292 | 75.840 | 224.940 | 105.429 | 1.00 | 97.10 | 6 |
| ATOM | 1828 | OG | SER | 292 | 74.624 | 225.670 | 105.452 | 1.00 | 97.55 | 8 |
| ATOM | 1829 | C | SER | 292 | 76.741 | 226.058 | 103.382 | 1.00 | 99.29 | 6 |
| ATOM | 1830 | O | SER | 292 | 77.561 | 225.684 | 102.543 | 1.00 | 100.00 | 8 |
| ATOM | 1831 | N | GLU | 293 | 75.638 | 226.736 | 103.076 | 1.00 | 95.37 | 7 |
| ATOM | 1832 | CA | GLU | 293 | 75.272 | 227.067 | 101.704 | 1.00 | 94.55 | 6 |
| ATOM | 1833 | CB | GLU | 293 | 73.899 | 227.758 | 101.680 | 1.00 | 99.13 | 6 |
| ATOM | 1834 | CG | GLU | 293 | 73.865 | 229.208 | 102.223 | 1.00 | 100.00 | 6 |
| ATOM | 1835 | CD | GLU | 293 | 74.064 | 229.316 | 103.739 | 1.00 | 100.00 | 6 |
| ATOM | 1836 | OE1 | GLU | 293 | 75.219 | 229.168 | 104.208 | 1.00 | 100.00 | 8 |
| ATOM | 1837 | OE2 | GLU | 293 | 73.065 | 229.570 | 104.456 | 1.00 | 100.00 | 8 |
| ATOM | 1838 | C | GLU | 293 | 76.312 | 227.930 | 100.972 | 1.00 | 92.35 | 6 |
| ATOM | 1839 | O | GLU | 293 | 76.289 | 228.022 | 99.743 | 1.00 | 89.41 | 8 |
| ATOM | 1840 | N | VAL | 294 | 77.213 | 228.554 | 101.730 | 1.00 | 91.83 | 7 |
| ATOM | 1841 | CA | VAL | 294 | 78.267 | 229.412 | 101.167 | 1.00 | 91.69 | 6 |
| ATOM | 1842 | CB | VAL | 294 | 78.742 | 230.473 | 102.209 | 1.00 | 94.30 | 6 |
| ATOM | 1843 | CG1 | VAL | 294 | 79.813 | 231.395 | 101.611 | 1.00 | 93.85 | 6 |
| ATOM | 1844 | CG2 | VAL | 294 | 77.557 | 231.294 | 102.691 | 1.00 | 96.91 | 6 |
| ATOM | 1845 | C | VAL | 294 | 79.480 | 228.621 | 100.664 | 1.00 | 87.73 | 6 |
| ATOM | 1846 | O | VAL | 294 | 79.968 | 227.714 | 101.344 | 1.00 | 86.99 | 8 |
| ATOM | 1847 | N | SER | 295 | 79.994 | 229.027 | 99.505 | 1.00 | 83.60 | 7 |
| ATOM | 1848 | CA | SER | 295 | 81.151 | 228.399 | 98.857 | 1.00 | 85.59 | 6 |
| ATOM | 1849 | CB | SER | 295 | 81.595 | 229.275 | 97.679 | 1.00 | 87.62 | 6 |
| ATOM | 1850 | OG | SER | 295 | 82.783 | 228.784 | 97.077 | 1.00 | 90.76 | 8 |
| ATOM | 1851 | C | SER | 295 | 82.380 | 228.046 | 99.721 | 1.00 | 84.88 | 6 |
| ATOM | 1852 | O | SER | 295 | 82.494 | 228.458 | 100.875 | 1.00 | 82.15 | 8 |
| ATOM | 1853 | N | GLU | 296 | 83.282 | 227.254 | 99.143 | 1.00 | 86.09 | 7 |
| ATOM | 1854 | CA | GLU | 296 | 84.507 | 226.828 | 99.817 | 1.00 | 88.44 | 6 |
| ATOM | 1855 | CB | GLU | 296 | 84.747 | 225.331 | 99.619 | 1.00 | 91.27 | 6 |
| ATOM | 1856 | CG | GLU | 296 | 84.934 | 224.532 | 100.914 | 1.00 | 95.23 | 6 |
| ATOM | 1857 | CD | GLU | 296 | 86.173 | 224.933 | 101.707 | 1.00 | 98.21 | 6 |
| ATOM | 1858 | OE1 | GLU | 296 | 87.239 | 225.177 | 101.099 | 1.00 | 97.65 | 8 |
| ATOM | 1859 | OE2 | GLU | 296 | 86.079 | 224.990 | 102.953 | 1.00 | 100.00 | |
| ATOM | 1860 | C | GLU | 296 | 85.682 | 227.611 | 99.245 | 1.00 | 88.98 | 6 |
| ATOM | 1861 | O | GLU | 296 | 86.727 | 227.749 | 99.890 | 1.00 | 89.20 | 8 |
| ATOM | 1862 | N | GLU | 297 | 85.503 | 228.100 | 98.017 | 1.00 | 89.53 | 7 |
| ATOM | 1863 | CA | GLU | 297 | 86.521 | 228.883 | 97.314 | 1.00 | 87.42 | 6 |
| ATOM | 1864 | CB | GLU | 297 | 86.072 | 229.171 | 95.881 | 1.00 | 86.30 | 6 |
| ATOM | 1865 | CG | GLU | 297 | 86.965 | 230.157 | 95.143 | 1.00 | 88.87 | 6 |
| ATOM | 1866 | CD | GLU | 297 | 86.231 | 230.954 | 94.071 | 1.00 | 91.65 | 6 |
| ATOM | 1867 | OE1 | GLU | 297 | 86.914 | 231.502 | 93.180 | 1.00 | 92.26 | 8 |
| ATOM | 1868 | OE2 | GLU | 297 | 84.982 | 231.050 | 94.123 | 1.00 | 90.45 | 8 |
| ATOM | 1869 | C | GLU | 297 | 86.691 | 230.202 | 98.053 | 1.00 | 85.34 | 6 |
| ATOM | 1870 | O | GLU | 297 | 87.804 | 230.693 | 98.224 | 1.00 | 84.25 | 8 |
| ATOM | 1871 | N | VAL | 298 | 85.562 | 230.749 | 98.494 | 1.00 | 84.38 | 7 |
| ATOM | 1872 | CA | VAL | 298 | 85.502 | 232.008 | 99.227 | 1.00 | 86.45 | 6 |
| ATOM | 1873 | CB | VAL | 298 | 84.037 | 232.461 | 99.406 | 1.00 | 89.83 | 6 |
| ATOM | 1874 | CG1 | VAL | 298 | 83.976 | 233.919 | 99.859 | 1.00 | 93.97 | 6 |
| ATOM | 1875 | CG2 | VAL | 298 | 83.260 | 232.254 | 98.122 | 1.00 | 92.45 | 6 |
| ATOM | 1876 | C | VAL | 298 | 86.114 | 231.862 | 100.621 | 1.00 | 86.34 | 6 |
| ATOM | 1877 | O | VAL | 298 | 86.588 | 232.835 | 101.209 | 1.00 | 86.25 | 8 |
| ATOM | 1878 | N | LYS | 299 | 86.074 | 230.643 | 101.149 | 1.00 | 87.55 | 7 |
| ATOM | 1879 | CA | LYS | 299 | 86.614 | 230.350 | 102.469 | 1.00 | 85.81 | 6 |
| ATOM | 1880 | CB | LYS | 299 | 86.085 | 229.002 | 102.982 | 1.00 | 85.19 | 6 |
| ATOM | 1881 | CG | LYS | 299 | 84.563 | 228.983 | 103.128 | 1.00 | 87.80 | 6 |
| ATOM | 1882 | CD | LYS | 299 | 84.018 | 227.695 | 103.729 | 1.00 | 93.61 | 6 |
| ATOM | 1883 | CE | LYS | 299 | 82.493 | 227.766 | 103.861 | 1.00 | 97.09 | 6 |
| ATOM | 1884 | NZ | LYS | 299 | 81.884 | 226.597 | 104.563 | 1.00 | 98.33 | 7 |
| ATOM | 1885 | C | LYS | 299 | 88.133 | 230.390 | 102.471 | 1.00 | 84.77 | 6 |
| ATOM | 1886 | O | LYS | 299 | 88.728 | 230.782 | 103.472 | 1.00 | 84.87 | 8 |
| ATOM | 1887 | N | MET | 300 | 88.754 | 230.019 | 101.349 | 1.00 | 83.67 | 7 |
| ATOM | 1888 | CA | MET | 300 | 90.220 | 230.025 | 101.239 | 1.00 | 83.82 | 6 |
| ATOM | 1889 | CB | MET | 300 | 90.673 | 229.485 | 99.875 | 1.00 | 91.34 | 6 |
| ATOM | 1890 | CG | MET | 300 | 92.173 | 229.730 | 99.528 | 1.00 | 98.50 | 6 |
| ATOM | 1891 | SD | MET | 300 | 92.674 | 231.467 | 99.102 | 1.00 | 100.00 | 16 |
| ATOM | 1892 | CE | MET | 300 | 93.082 | 231.332 | 97.329 | 1.00 | 93.54 | 6 |
| ATOM | 1893 | C | MET | 300 | 90.748 | 231.443 | 101.413 | 1.00 | 78.22 | 6 |
| ATOM | 1894 | O | MET | 300 | 91.868 | 231.656 | 101.892 | 1.00 | 79.06 | 8 |
| ATOM | 1895 | N | LEU | 301 | 89.921 | 232.398 | 101.000 | 1.00 | 71.14 | 7 |
| ATOM | 1896 | CA | LEU | 301 | 90.221 | 233.821 | 101.069 | 1.00 | 63.05 | 6 |
| ATOM | 1897 | CB | LEU | 301 | 89.171 | 234.566 | 100.269 | 1.00 | 59.75 | 6 |
| ATOM | 1898 | CG | LEU | 301 | 89.650 | 235.763 | 99.485 | 1.00 | 61.24 | 6 |
| ATOM | 1899 | CD1 | LEU | 301 | 91.117 | 235.598 | 99.091 | 1.00 | 59.15 | 6 |
| ATOM | 1900 | CD2 | LEU | 301 | 88.731 | 235.904 | 98.277 | 1.00 | 63.03 | 6 |
| ATOM | 1901 | C | LEU | 301 | 90.211 | 234.320 | 102.502 | 1.00 | 61.02 | 6 |
| ATOM | 1902 | O | LEU | 301 | 91.184 | 234.904 | 102.980 | 1.00 | 57.69 | 8 |
| ATOM | 1903 | N | ILE | 302 | 89.104 | 234.075 | 103.190 | 1.00 | 58.87 | 7 |
| ATOM | 1904 | CA | ILE | 302 | 88.971 | 234.496 | 104.569 | 1.00 | 61.17 | 6 |
| ATOM | 1905 | CB | ILE | 302 | 87.608 | 234.110 | 105.122 | 1.00 | 58.05 | 6 |
| ATOM | 1906 | CG2 | ILE | 302 | 87.491 | 234.540 | 106.570 | 1.00 | 51.39 | 6 |
| ATOM | 1907 | CG1 | ILE | 302 | 86.519 | 234.776 | 104.282 | 1.00 | 62.26 | 6 |
| ATOM | 1908 | CD1 | ILE | 302 | 85.168 | 234.091 | 104.351 | 1.00 | 67.55 | 6 |
| ATOM | 1909 | C | ILE | 302 | 90.071 | 233.855 | 105.406 | 1.00 | 66.49 | |
| ATOM | 1910 | O | ILE | 302 | 90.503 | 234.419 | 106.404 | 1.00 | 69.43 | 8 |
| ATOM | 1911 | N | ARG | 303 | 90.544 | 232.694 | 104.958 | 1.00 | 71.10 | 7 |
| ATOM | 1912 | CA | ARG | 303 | 91.597 | 231.957 | 105.643 | 1.00 | 69.40 | 6 |
| ATOM | 1913 | CB | ARG | 303 | 91.552 | 230.485 | 105.243 | 1.00 | 71.07 | 6 |
| ATOM | 1914 | CG | ARG | 303 | 90.412 | 229.716 | 105.894 | 1.00 | 75.29 | 6 |
| ATOM | 1915 | CD | ARG | 303 | 90.375 | 228.250 | 105.454 | 1.00 | 76.49 | 6 |
| ATOM | 1916 | NE | ARG | 303 | 89.275 | 227.523 | 106.088 | 1.00 | 77.93 | 7 |
| ATOM | 1917 | CZ | ARG | 303 | 88.691 | 226.440 | 105.587 | 1.00 | 77.15 | 6 |
| ATOM | 1918 | NH1 | ARG | 303 | 89.092 | 225.929 | 104.433 | 1.00 | 78.05 | 7 |
| ATOM | 1919 | NH2 | ARG | 303 | 87.690 | 225.873 | 106.240 | 1.00 | 80.74 | 7 |
| ATOM | 1920 | C | ARG | 303 | 92.979 | 232.543 | 105.393 | 1.00 | 69.41 | 6 |
| ATOM | 1921 | O | ARG | 303 | 93.776 | 232.673 | 106.326 | 1.00 | 67.01 | 8 |
| ATOM | 1922 | N | ASN | 304 | 93.262 | 232.899 | 104.143 | 1.00 | 71.15 | 7 |
| ATOM | 1923 | CA | ASN | 304 | 94.565 | 233.478 | 103.797 | 1.00 | 76.89 | 6 |
| ATOM | 1924 | CB | ASN | 304 | 94.780 | 233.516 | 102.279 | 1.00 | 77.87 | 6 |
| ATOM | 1925 | CG | ASN | 304 | 95.230 | 232.177 | 101.713 | 1.00 | 80.94 | 6 |
| ATOM | 1926 | OD1 | ASN | 304 | 95.138 | 231.950 | 100.505 | 1.00 | 83.74 | 8 |
| ATOM | 1927 | ND2 | ASN | 304 | 95.740 | 231.293 | 102.575 | 1.00 | 76.87 | 7 |
| ATOM | 1928 | C | ASN | 304 | 94.712 | 234.885 | 104.378 | 1.00 | 76.89 | 6 |
| ATOM | 1929 | O | ASN | 304 | 95.826 | 235.334 | 104.696 | 1.00 | 77.32 | 8 |
| ATOM | 1930 | N | LEU | 305 | 93.577 | 235.572 | 104.504 | 1.00 | 72.94 | 7 |
| ATOM | 1931 | CA | LEU | 305 | 93.539 | 236.925 | 105.044 | 1.00 | 65.51 | 6 |
| ATOM | 1932 | CB | LEU | 305 | 92.222 | 237.606 | 104.665 | 1.00 | 61.03 | 6 |
| ATOM | 1933 | CG | LEU | 305 | 92.084 | 238.318 | 103.321 | 1.00 | 58.86 | 6 |
| ATOM | 1934 | CD1 | LEU | 305 | 92.633 | 237.498 | 102.173 | 1.00 | 59.31 | 6 |
| ATOM | 1935 | CD2 | LEU | 305 | 90.623 | 238.619 | 103.100 | 1.00 | 61.97 | 6 |
| ATOM | 1936 | C | LEU | 305 | 93.656 | 236.841 | 106.559 | 1.00 | 62.51 | 6 |
| ATOM | 1937 | O | LEU | 305 | 94.436 | 237.574 | 107.173 | 1.00 | 55.05 | 8 |
| ATOM | 1938 | N | LEU | 306 | 92.894 | 235.910 | 107.139 | 1.00 | 61.43 | 7 |
| ATOM | 1939 | CA | LEU | 306 | 92.871 | 235.681 | 108.582 | 1.00 | 60.96 | 6 |
| ATOM | 1940 | CB | LEU | 306 | 91.522 | 235.117 | 109.019 | 1.00 | 50.96 | 6 |
| ATOM | 1941 | CG | LEU | 306 | 90.481 | 236.052 | 109.610 | 1.00 | 49.81 | 6 |
| ATOM | 1942 | CD1 | LEU | 306 | 90.914 | 237.500 | 109.493 | 1.00 | 47.89 | 6 |
| ATOM | 1943 | CD2 | LEU | 306 | 89.158 | 235.798 | 108.915 | 1.00 | 41.87 | 6 |
| ATOM | 1944 | C | LEU | 306 | 93.985 | 234.791 | 109.128 | 1.00 | 64.54 | 6 |
| ATOM | 1945 | O | LEU | 306 | 93.730 | 233.916 | 109.960 | 1.00 | 67.76 | 8 |
| ATOM | 1946 | N | LYS | 307 | 95.204 | 234.999 | 108.634 | 1.00 | 65.30 | 7 |
| ATOM | 1947 | CA | LYS | 307 | 96.369 | 234.237 | 109.068 | 1.00 | 64.60 | 6 |
| ATOM | 1948 | CB | LYS | 307 | 97.432 | 234.239 | 107.967 | 1.00 | 65.63 | 6 |
| ATOM | 1949 | CG | LYS | 307 | 97.119 | 233.323 | 106.792 | 1.00 | 63.42 | 6 |
| ATOM | 1950 | CD | LYS | 307 | 97.448 | 231.877 | 107.143 | 1.00 | 67.43 | 6 |
| ATOM | 1951 | CE | LYS | 307 | 97.116 | 230.900 | 106.025 | 1.00 | 65.67 | 6 |
| ATOM | 1952 | NZ | LYS | 307 | 97.505 | 229.531 | 106.450 | 1.00 | 65.81 | 7 |
| ATOM | 1953 | C | LYS | 307 | 96.884 | 234.962 | 110.314 | 1.00 | 63.50 | 6 |
| ATOM | 1954 | O | LYS | 307 | 96.854 | 236.182 | 110.368 | 1.00 | 64.53 | 8 |
| ATOM | 1955 | N | THR | 308 | 97.330 | 234.228 | 111.324 | 1.00 | 63.07 | 7 |
| ATOM | 1956 | CA | THR | 308 | 97.818 | 234.890 | 112.525 | 1.00 | 62.57 | 6 |
| ATOM | 1957 | CB | THR | 308 | 97.850 | 233.944 | 113.769 | 1.00 | 67.53 | 6 |
| ATOM | 1958 | OG1 | THR | 308 | 99.080 | 233.203 | 113.799 | 1.00 | 70.53 | 8 |
| ATOM | 1959 | CG2 | THR | 308 | 96.652 | 232.984 | 113.764 | 1.00 | 61.84 | 6 |
| ATOM | 1960 | C | THR | 308 | 99.186 | 235.517 | 112.331 | 1.00 | 60.55 | 6 |
| ATOM | 1961 | O | THR | 308 | 99.553 | 236.452 | 113.040 | 1.00 | 58.58 | 8 |
| ATOM | 1962 | N | ALA | 309 | 99.955 | 234.987 | 111.388 | 1.00 | 63.03 | 7 |
| ATOM | 1963 | CA | ALA | 309 | 101.289 | 235.534 | 111.140 | 1.00 | 68.60 | 6 |
| ATOM | 1964 | CB | ALA | 309 | 102.236 | 234.452 | 110.577 | 1.00 | 57.95 | 6 |
| ATOM | 1965 | C | ALA | 309 | 101.236 | 236.742 | 110.204 | 1.00 | 69.72 | 6 |
| ATOM | 1966 | O | ALA | 309 | 100.473 | 236.747 | 109.223 | 1.00 | 73.60 | 8 |
| ATOM | 1967 | N | ALA | 310 | 101.984 | 237.789 | 110.556 | 1.00 | 65.13 | 7 |
| ATOM | 1968 | CA | ALA | 310 | 102.028 | 238.995 | 109.742 | 1.00 | 60.27 | 6 |
| ATOM | 1969 | CB | ALA | 310 | 103.065 | 239.957 | 110.291 | 1.00 | 63.72 | 6 |
| ATOM | 1970 | C | ALA | 310 | 102.406 | 238.504 | 108.338 | 1.00 | 58.37 | 6 |
| ATOM | 1971 | O | ALA | 310 | 101.584 | 238.576 | 107.422 | 1.00 | 56.83 | 8 |
| ATOM | 1972 | N | ALA | 311 | 103.599 | 237.907 | 108.198 | 1.00 | 55.01 | 7 |
| ATOM | 1973 | CA | ALA | 311 | 104.070 | 237.375 | 106.908 | 1.00 | 51.77 | 6 |
| ATOM | 1974 | CB | ALA | 311 | 105.453 | 236.778 | 107.069 | 1.00 | 28.89 | 6 |
| ATOM | 1975 | C | ALA | 311 | 103.056 | 236.271 | 106.564 | 1.00 | 56.29 | 6 |
| ATOM | 1976 | O | ALA | 311 | 102.069 | 236.104 | 107.287 | 1.00 | 61.54 | 8 |
| ATOM | 1977 | N | ALA | 312 | 103.230 | 235.542 | 105.469 | 1.00 | 58.24 | 7 |
| ATOM | 1978 | CA | ALA | 312 | 102.261 | 234.473 | 105.136 | 1.00 | 62.72 | 6 |
| ATOM | 1979 | CB | ALA | 312 | 102.285 | 233.375 | 106.227 | 1.00 | 61.28 | 6 |
| ATOM | 1980 | C | ALA | 312 | 100.801 | 234.892 | 104.864 | 1.00 | 62.04 | 6 |
| ATOM | 1981 | O | ALA | 312 | 100.050 | 234.151 | 104.220 | 1.00 | 57.84 | 8 |
| ATOM | 1982 | N | ARG | 313 | 100.396 | 236.049 | 105.387 | 1.00 | 62.33 | 7 |
| ATOM | 1983 | CA | ARG | 313 | 99.043 | 236.557 | 105.201 | 1.00 | 58.23 | 6 |
| ATOM | 1984 | CB | ARG | 313 | 98.718 | 237.610 | 106.266 | 1.00 | 52.76 | 6 |
| ATOM | 1985 | CG | ARG | 313 | 97.249 | 237.978 | 106.338 | 1.00 | 44.89 | 6 |
| ATOM | 1986 | CD | ARG | 313 | 96.978 | 239.079 | 107.360 | 1.00 | 43.94 | 6 |
| ATOM | 1987 | NE | ARG | 313 | 96.960 | 238.630 | 108.748 | 1.00 | 34.75 | 7 |
| ATOM | 1988 | CZ | ARG | 313 | 97.746 | 239.124 | 109.702 | 1.00 | 39.40 | 6 |
| ATOM | 1989 | NH1 | ARG | 313 | 98.613 | 240.088 | 109.439 | 1.00 | 37.30 | 7 |
| ATOM | 1990 | NH2 | ARG | 313 | 97.705 | 238.619 | 110.921 | 1.00 | 42.01 | 7 |
| ATOM | 1991 | C | ARG | 313 | 99.004 | 237.185 | 103.821 | 1.00 | 60.40 | 6 |
| ATOM | 1992 | O | ARG | 313 | 99.933 | 237.909 | 103.437 | 1.00 | 63.00 | 8 |
| ATOM | 1993 | N | MET | 314 | 97.946 | 236.875 | 103.072 | 1.00 | 60.87 | 7 |
| ATOM | 1994 | CA | MET | 314 | 97.742 | 237.388 | 101.712 | 1.00 | 59.63 | 6 |
| ATOM | 1995 | CB | MET | 314 | 96.308 | 237.061 | 101.262 | 1.00 | 54.24 | 6 |
| ATOM | 1996 | CG | MET | 314 | 95.992 | 237.476 | 99.839 | 1.00 | 56.69 | 6 |
| ATOM | 1997 | SD | MET | 314 | 94.404 | 236.858 | 99.263 | 1.00 | 65.96 | 16 |
| ATOM | 1998 | CE | MET | 314 | 94.890 | 235.270 | 98.573 | 1.00 | 71.51 | 6 |
| ATOM | 1999 | C | MET | 314 | 98.015 | 238.897 | 101.550 | 1.00 | 61.09 | 6 |
| ATOM | 2000 | O | MET | 314 | 97.755 | 239.685 | 102.472 | 1.00 | 63.27 | 8 |
| ATOM | 2001 | N | THR | 315 | 98.572 | 239.284 | 100.397 | 1.00 | 58.39 | 7 |
| ATOM | 2002 | CA | THR | 315 | 98.878 | 240.692 | 100.107 | 1.00 | 55.90 | 6 |
| ATOM | 2003 | CB | THR | 315 | 100.172 | 240.853 | 99.249 | 1.00 | 57.21 | 6 |
| ATOM | 2004 | OG1 | THR | 315 | 99.922 | 240.486 | 97.881 | 1.00 | 57.00 | 8 |
| ATOM | 2005 | CG2 | THR | 315 | 101.308 | 240.008 | 99.813 | 1.00 | 52.24 | 6 |
| ATOM | 2006 | C | THR | 315 | 97.693 | 241.337 | 99.361 | 1.00 | 56.00 | 6 |
| ATOM | 2007 | O | THR | 315 | 96.795 | 240.626 | 98.898 | 1.00 | 56.57 | 8 |
| ATOM | 2008 | N | ILE | 316 | 97.673 | 242.670 | 99.257 | 1.00 | 56.10 | 7 |
| ATOM | 2009 | CA | ILE | 316 | 96.572 | 243.361 | 98.559 | 1.00 | 52.69 | 6 |
| ATOM | 2010 | CB | ILE | 316 | 96.618 | 244.922 | 98.735 | 1.00 | 46.51 | 6 |
| ATOM | 2011 | CG2 | ILE | 316 | 97.711 | 245.545 | 97.882 | 1.00 | 42.44 | 6 |
| ATOM | 2012 | CG1 | ILE | 316 | 95.304 | 245.542 | 98.271 | 1.00 | 32.71 | 6 |
| ATOM | 2013 | CD1 | ILE | 316 | 94.130 | 245.091 | 99.068 | 1.00 | 36.32 | 6 |
| ATOM | 2014 | C | ILE | 316 | 96.559 | 243.056 | 97.068 | 1.00 | 52.94 | 6 |
| ATOM | 2015 | O | ILE | 316 | 95.498 | 242.975 | 96.448 | 1.00 | 53.27 | 8 |
| ATOM | 2016 | N | THR | 317 | 97.751 | 242.867 | 96.515 | 1.00 | 51.84 | 7 |
| ATOM | 2017 | CA | THR | 317 | 97.913 | 242.575 | 95.101 | 1.00 | 53.59 | 6 |
| ATOM | 2018 | CB | THR | 317 | 99.381 | 242.692 | 94.709 | 1.00 | 54.78 | 6 |
| ATOM | 2019 | OG1 | THR | 317 | 100.054 | 243.558 | 95.646 | 1.00 | 53.23 | 8 |
| ATOM | 2020 | CG2 | THR | 317 | 99.503 | 243.240 | 93.290 | 1.00 | 48.84 | 6 |
| ATOM | 2021 | C | THR | 317 | 97.401 | 241.182 | 94.777 | 1.00 | 55.92 | 6 |
| ATOM | 2022 | O | THR | 317 | 96.935 | 240.922 | 93.671 | 1.00 | 54.42 | 8 |
| ATOM | 2023 | N | GLU | 318 | 97.497 | 240.294 | 95.761 | 1.00 | 61.74 | 7 |
| ATOM | 2024 | CA | GLU | 318 | 97.048 | 238.920 | 95.613 | 1.00 | 66.53 | 6 |
| ATOM | 2025 | CB | GLU | 318 | 97.812 | 237.995 | 96.575 | 1.00 | 67.74 | 6 |
| ATOM | 2026 | CG | GLU | 318 | 99.322 | 237.900 | 96.298 | 1.00 | 66.05 | 6 |
| ATOM | 2027 | CD | GLU | 318 | 100.045 | 236.885 | 97.187 | 1.00 | 71.37 | 6 |
| ATOM | 2028 | OE1 | GLU | 318 | 99.979 | 236.998 | 98.438 | 1.00 | 66.66 | 8 |
| ATOM | 2029 | OE2 | GLU | 318 | 100.700 | 235.981 | 96.622 | 1.00 | 72.71 | 8 |
| ATOM | 2030 | C | GLU | 318 | 95.544 | 238.861 | 95.881 | 1.00 | 68.15 | 6 |
| ATOM | 2031 | O | GLU | 318 | 94.828 | 238.049 | 95.290 | 1.00 | 69.31 | 8 |
| ATOM | 2032 | N | PHE | 319 | 95.068 | 239.743 | 96.759 | 1.00 | 68.68 | 7 |
| ATOM | 2033 | CA | PHE | 319 | 93.653 | 239.794 | 97.102 | 1.00 | 68.65 | 6 |
| ATOM | 2034 | CB | PHE | 319 | 93.405 | 240.792 | 98.249 | 1.00 | 62.07 | 6 |
| ATOM | 2035 | CG | PHE | 319 | 91.947 | 240.908 | 98.661 | 1.00 | 59.39 | 6 |
| ATOM | 2036 | CD1 | PHE | 319 | 91.063 | 241.715 | 97.949 | 1.00 | 55.67 | 6 |
| ATOM | 2037 | CD2 | PHE | 319 | 91.457 | 240.199 | 99.752 | 1.00 | 54.87 | 6 |
| ATOM | 2038 | CE1 | PHE | 319 | 89.730 | 241.808 | 98.316 | 1.00 | 49.32 | 6 |
| ATOM | 2039 | CE2 | PHE | 319 | 90.122 | 240.294 | 100.118 | 1.00 | 51.67 | 6 |
| ATOM | 2040 | CZ | PHE | 319 | 89.261 | 241.099 | 99.396 | 1.00 | 46.18 | 6 |
| ATOM | 2041 | C | PHE | 319 | 92.878 | 240.236 | 95.873 | 1.00 | 71.46 | 6 |
| ATOM | 2042 | O | PHE | 319 | 91.898 | 239.609 | 95.486 | 1.00 | 72.12 | 8 |
| ATOM | 2043 | N | MET | 320 | 93.359 | 241.306 | 95.251 | 1.00 | 74.50 | 7 |
| ATOM | 2044 | CA | MET | 320 | 92.736 | 241.868 | 94.067 | 1.00 | 76.81 | 6 |
| ATOM | 2045 | CB | MET | 320 | 93.363 | 243.229 | 93.756 | 1.00 | 78.28 | 6 |
| ATOM | 2046 | CG | MET | 320 | 92.338 | 244.342 | 93.529 | 1.00 | 80.53 | 6 |
| ATOM | 2047 | SD | MET | 320 | 91.087 | 244.456 | 94.831 | 1.00 | 77.61 | 16 |
| ATOM | 2048 | CE | MET | 320 | 92.080 | 244.892 | 96.188 | 1.00 | 80.25 | 6 |
| ATOM | 2049 | C | MET | 320 | 92.757 | 240.970 | 92.840 | 1.00 | 78.75 | 6 |
| ATOM | 2050 | O | MET | 320 | 91.973 | 241.174 | 91.915 | 1.00 | 80.84 | 8 |
| ATOM | 2051 | N | ASN | 321 | 93.650 | 239.983 | 92.828 | 1.00 | 80.16 | 7 |
| ATOM | 2052 | CA | ASN | 321 | 93.754 | 239.061 | 91.700 | 1.00 | 79.55 | 6 |
| ATOM | 2053 | CB | ASN | 321 | 95.211 | 238.780 | 91.373 | 1.00 | 79.09 | 6 |
| ATOM | 2054 | CG | ASN | 321 | 95.758 | 239.743 | 90.364 | 1.00 | 81.54 | 6 |
| ATOM | 2055 | OD1 | ASN | 321 | 95.105 | 240.041 | 89.367 | 1.00 | 85.07 | 8 |
| ATOM | 2056 | ND2 | ASN | 321 | 96.961 | 240.241 | 90.608 | 1.00 | 84.63 | 7 |
| ATOM | 2057 | C | ASN | 321 | 92.990 | 237.753 | 91.873 | 1.00 | 81.16 | 6 |
| ATOM | 2058 | O | ASN | 321 | 92.842 | 236.985 | 90.922 | 1.00 | 82.37 | 8 |
| ATOM | 2059 | N | HIS | 322 | 92.525 | 237.488 | 93.088 | 1.00 | 83.32 | 7 |
| ATOM | 2060 | CA | HIS | 322 | 91.776 | 236.269 | 93.359 | 1.00 | 86.48 | 6 |
| ATOM | 2061 | CB | HIS | 322 | 91.506 | 236.131 | 94.861 | 1.00 | 87.65 | 6 |
| ATOM | 2062 | CG | HIS | 322 | 90.691 | 234.931 | 95.230 | 1.00 | 87.78 | 6 |
| ATOM | 2063 | CD2 | HIS | 322 | 90.948 | 233.905 | 96.074 | 1.00 | 90.78 | 6 |
| ATOM | 2064 | ND1 | HIS | 322 | 89.430 | 234.698 | 94.724 | 1.00 | 88.02 | 7 |
| ATOM | 2065 | CE1 | HIS | 322 | 88.947 | 233.583 | 95.238 | 1.00 | 89.57 | 6 |
| ATOM | 2066 | NE2 | HIS | 322 | 89.849 | 233.082 | 96.062 | 1.00 | 92.43 | 7 |
| ATOM | 2067 | C | HIS | 322 | 90.466 | 236.366 | 92.576 | 1.00 | 87.09 | 6 |
| ATOM | 2068 | O | HIS | 322 | 89.874 | 237.447 | 92.471 | 1.00 | 86.22 | 8 |
| ATOM | 2069 | N | PRO | 323 | 90.016 | 235.244 | 91.986 | 1.00 | 87.32 | 7 |
| ATOM | 2070 | CD | PRO | 323 | 90.742 | 233.967 | 91.885 | 1.00 | 83.40 | 6 |
| ATOM | 2071 | CA | PRO | 323 | 88.780 | 235.185 | 91.200 | 1.00 | 86.59 | 6 |
| ATOM | 2072 | CB | PRO | 323 | 88.692 | 233.711 | 90.838 | 1.00 | 84.41 | 6 |
| ATOM | 2073 | CG | PRO | 323 | 90.129 | 233.361 | 90.654 | 1.00 | 82.75 | 6 |
| ATOM | 2074 | C | PRO | 323 | 87.507 | 235.701 | 91.875 | 1.00 | 86.16 | 6 |
| ATOM | 2075 | O | PRO | 323 | 86.775 | 236.482 | 91.272 | 1.00 | 85.38 | 8 |
| ATOM | 2076 | N | TRP | 324 | 87.252 | 235.295 | 93.119 | 1.00 | 86.13 | 7 |
| ATOM | 2077 | CA | TRP | 324 | 86.053 | 235.743 | 93.832 | 1.00 | 85.95 | 6 |
| ATOM | 2078 | CB | TRP | 324 | 86.062 | 235.281 | 95.294 | 1.00 | 82.18 | 6 |
| ATOM | 2079 | CG | TRP | 324 | 84.729 | 235.477 | 95.975 | 1.00 | 80.10 | 6 |
| ATOM | 2080 | CD2 | TRP | 324 | 84.411 | 236.424 | 97.002 | 1.00 | 83.28 | 6 |
| ATOM | 2081 | CE2 | TRP | 324 | 83.051 | 236.228 | 97.345 | 1.00 | 82.38 | 6 |
| ATOM | 2082 | CE3 | TRP | 324 | 85.137 | 237.422 | 97.667 | 1.00 | 86.77 | 6 |
| ATOM | 2083 | CD1 | TRP | 324 | 83.583 | 234.774 | 95.739 | 1.00 | 80.05 | 6 |
| ATOM | 2084 | NE1 | TRP | 324 | 82.573 | 235.215 | 96.557 | 1.00 | 78.64 | 7 |
| ATOM | 2085 | CZ2 | TRP | 324 | 82.402 | 236.993 | 98.327 | 1.00 | 82.54 | 6 |
| ATOM | 2086 | CZ3 | TRP | 324 | 84.487 | 238.189 | 98.648 | 1.00 | 84.53 | 6 |
| ATOM | 2087 | CH2 | TRP | 324 | 83.136 | 237.966 | 98.964 | 1.00 | 82.88 | 6 |
| ATOM | 2088 | C | TRP | 324 | 85.878 | 237.259 | 93.783 | 1.00 | 87.44 | 6 |
| ATOM | 2089 | O | TRP | 324 | 84.758 | 237.751 | 93.627 | 1.00 | 85.83 | 8 |
| ATOM | 2090 | N | ILE | 325 | 86.993 | 237.982 | 93.912 | 1.00 | 87.47 | 7 |
| ATOM | 2091 | CA | ILE | 325 | 87.000 | 239.445 | 93.888 | 1.00 | 83.93 | 6 |
| ATOM | 2092 | CB | ILE | 325 | 88.282 | 240.016 | 94.568 | 1.00 | 75.44 | 6 |
| ATOM | 2093 | CG2 | ILE | 325 | 88.310 | 241.524 | 94.472 | 1.00 | 71.00 | 6 |
| ATOM | 2094 | CG1 | ILE | 325 | 88.373 | 239.566 | 96.029 | 1.00 | 65.12 | 6 |
| ATOM | 2095 | CD1 | ILE | 325 | 87.279 | 240.088 | 96.893 | 1.00 | 59.73 | 6 |
| ATOM | 2096 | C | ILE | 325 | 86.937 | 239.938 | 92.443 | 1.00 | 88.32 | 6 |
| ATOM | 2097 | O | ILE | 325 | 85.976 | 240.599 | 92.041 | 1.00 | 93.04 | 8 |
| ATOM | 2098 | N | MET | 326 | 87.960 | 239.592 | 91.670 | 1.00 | 88.36 | 7 |
| ATOM | 2099 | CA | MET | 326 | 88.058 | 239.979 | 90.271 | 1.00 | 92.38 | 6 |
| ATOM | 2100 | CB | MET | 326 | 89.508 | 239.830 | 89.823 | 1.00 | 93.38 | 6 |
| ATOM | 2101 | CG | MET | 326 | 89.740 | 240.014 | 88.340 | 1.00 | 96.92 | 6 |
| ATOM | 2102 | SD | MET | 326 | 91.466 | 239.719 | 87.936 | 1.00 | 100.00 | 16 |
| ATOM | 2103 | CE | MET | 326 | 91.967 | 241.392 | 87.385 | 1.00 | 100.00 | 6 |
| ATOM | 2104 | C | MET | 326 | 87.155 | 239.131 | 89.380 | 1.00 | 95.92 | 6 |
| ATOM | 2105 | O | MET | 326 | 87.380 | 237.929 | 89.240 | 1.00 | 99.69 | 8 |
| ATOM | 2106 | N | GLN | 327 | 86.148 | 239.752 | 88.765 | 1.00 | 97.73 | 7 |
| ATOM | 2107 | CA | GLN | 327 | 85.228 | 239.017 | 87.887 | 1.00 | 99.65 | 6 |
| ATOM | 2108 | CB | GLN | 327 | 84.477 | 237.924 | 88.675 | 1.00 | 96.03 | 6 |
| ATOM | 2109 | CG | GLN | 327 | 83.977 | 238.325 | 90.068 | 1.00 | 95.50 | 6 |
| ATOM | 2110 | CD | GLN | 327 | 82.881 | 239.376 | 90.050 | 1.00 | 95.94 | 6 |
| ATOM | 2111 | OE1 | GLN | 327 | 81.737 | 239.089 | 89.694 | 1.00 | 93.98 | 8 |
| ATOM | 2112 | NE2 | GLN | 327 | 83.225 | 240.601 | 90.448 | 1.00 | 91.15 | 7 |
| ATOM | 2113 | C | GLN | 327 | 84.216 | 239.837 | 87.070 | 1.00 | 100.00 | 6 |
| ATOM | 2114 | O | GLN | 327 | 83.933 | 239.498 | 85.907 | 1.00 | 100.00 | 8 |
| ATOM | 2115 | N | ALA | 328 | 83.695 | 240.910 | 87.680 | 1.00 | 100.00 | 7 |
| ATOM | 2116 | CA | ALA | 328 | 82.699 | 241.816 | 87.077 | 1.00 | 100.00 | 6 |
| ATOM | 2117 | CB | ALA | 328 | 82.999 | 242.097 | 85.595 | 1.00 | 100.00 | 6 |
| ATOM | 2118 | C | ALA | 328 | 81.316 | 241.170 | 87.241 | 1.00 | 100.00 | 6 |
| ATOM | 2119 | O | ALA | 328 | 80.362 | 241.821 | 87.661 | 1.00 | 98.40 | 8 |
| ATOM | 2120 | N | ALA | 329 | 81.251 | 239.869 | 86.954 | 1.00 | 100.00 | 7 |
| ATOM | 2121 | CA | ALA | 329 | 80.034 | 239.062 | 87.049 | 1.00 | 100.00 | 6 |
| ATOM | 2122 | CB | ALA | 329 | 78.997 | 239.528 | 86.015 | 1.00 | 100.00 | 6 |
| ATOM | 2123 | C | ALA | 329 | 80.422 | 237.598 | 86.791 | 1.00 | 100.00 | 6 |
| ATOM | 2124 | O | ALA | 329 | 80.806 | 237.247 | 85.667 | 1.00 | 100.00 | 8 |
| ATOM | 2125 | N | ALA | 330 | 80.356 | 236.764 | 87.836 | 1.00 | 100.00 | 7 |
| ATOM | 2126 | CA | ALA | 330 | 80.697 | 235.332 | 87.738 | 1.00 | 100.00 | 6 |
| ATOM | 2127 | CB | ALA | 330 | 82.150 | 235.149 | 87.265 | 1.00 | 100.00 | 6 |
| ATOM | 2128 | C | ALA | 330 | 80.483 | 234.581 | 89.055 | 1.00 | 98.79 | 6 |
| ATOM | 2129 | O | ALA | 330 | 80.353 | 233.354 | 89.065 | 1.00 | 95.99 | 8 |
| ATOM | 2130 | N | ALA | 331 | 80.494 | 235.322 | 90.163 | 1.00 | 99.07 | 7 |
| ATOM | 2131 | CA | ALA | 331 | 80.303 | 234.749 | 91.493 | 1.00 | 97.00 | 6 |
| ATOM | 2132 | CB | ALA | 331 | 80.692 | 235.766 | 92.566 | 1.00 | 93.98 | 6 |
| ATOM | 2133 | C | ALA | 331 | 78.854 | 234.329 | 91.675 | 1.00 | 96.04 | 6 |
| ATOM | 2134 | O | ALA | 331 | 77.952 | 234.942 | 91.111 | 1.00 | 96.34 | 8 |
| ATOM | 2135 | N | ALA | 332 | 78.633 | 233.281 | 92.459 | 1.00 | 95.51 | 7 |
| ATOM | 2136 | CA | ALA | 332 | 77.282 | 232.788 | 92.711 | 1.00 | 95.51 | 6 |
| ATOM | 2137 | CB | ALA | 332 | 77.336 | 231.537 | 93.590 | 1.00 | 93.26 | 6 |
| ATOM | 2138 | C | ALA | 332 | 76.439 | 233.860 | 93.394 | 1.00 | 95.31 | 6 |
| ATOM | 2139 | O | ALA | 332 | 76.960 | 234.669 | 94.152 | 1.00 | 100.00 | 8 |
| ATOM | 2140 | N | ALA | 333 | 75.146 | 233.895 | 93.090 | 1.00 | 94.01 | 7 |
| ATOM | 2141 | CA | ALA | 333 | 74.252 | 234.875 | 93.699 | 1.00 | 91.18 | 6 |
| ATOM | 2142 | CB | ALA | 333 | 73.240 | 235.360 | 92.690 | 1.00 | 89.00 | 6 |
| ATOM | 2143 | C | ALA | 333 | 73.552 | 234.221 | 94.900 | 1.00 | 91.02 | 6 |
| ATOM | 2144 | O | ALA | 333 | 72.571 | 234.753 | 95.427 | 1.00 | 84.80 | 8 |
| ATOM | 2145 | N | ALA | 334 | 74.112 | 233.084 | 95.331 | 1.00 | 94.02 | 7 |
| ATOM | 2146 | CA | ALA | 334 | 73.635 | 232.269 | 96.453 | 1.00 | 95.02 | 6 |
| ATOM | 2147 | CB | ALA | 334 | 74.770 | 231.379 | 96.974 | 1.00 | 90.08 | 6 |
| ATOM | 2148 | C | ALA | 334 | 72.994 | 233.042 | 97.602 | 1.00 | 96.31 | 6 |
| ATOM | 2149 | O | ALA | 334 | 73.604 | 233.236 | 98.656 | 1.00 | 95.05 | 8 |
| ATOM | 2150 | N | ALA | 335 | 71.725 | 233.394 | 97.392 | 1.00 | 97.89 | 7 |
| ATOM | 2151 | CA | ALA | 335 | 70.886 | 234.140 | 98.329 | 1.00 | 98.67 | 6 |
| ATOM | 2152 | CB | ALA | 335 | 69.426 | 233.725 | 98.167 | 1.00 | 95.01 | 6 |
| ATOM | 2153 | C | ALA | 335 | 71.280 | 234.096 | 99.800 | 1.00 | 99.36 | 6 |
| ATOM | 2154 | O | ALA | 335 | 71.326 | 233.033 | 100.424 | 1.00 | 99.14 | 8 |
| ATOM | 2155 | N | ALA | 336 | 71.576 | 235.281 | 100.324 | 1.00 | 100.00 | 7 |
| ATOM | 2156 | CA | ALA | 336 | 71.972 | 235.487 | 101.711 | 1.00 | 99.16 | 6 |
| ATOM | 2157 | CB | ALA | 336 | 73.497 | 235.496 | 101.833 | 1.00 | 95.56 | 6 |
| ATOM | 2158 | C | ALA | 336 | 71.384 | 236.838 | 102.154 | 1.00 | 100.00 | 6 |
| ATOM | 2159 | O | ALA | 336 | 71.726 | 237.356 | 103.220 | 1.00 | 100.00 | 8 |
| ATOM | 2160 | N | ALA | 337 | 70.486 | 237.387 | 101.324 | 1.00 | 100.00 | 6 |
| ATOM | 2161 | CA | ALA | 337 | 69.815 | 238.672 | 101.573 | 1.00 | 100.00 | 6 |
| ATOM | 2162 | CB | ALA | 337 | 68.731 | 238.927 | 100.516 | 1.00 | 100.00 | 6 |
| ATOM | 2163 | C | ALA | 337 | 69.225 | 238.738 | 102.970 | 1.00 | 100.00 | 6 |
| ATOM | 2164 | O | ALA | 337 | 69.547 | 239.639 | 103.743 | 1.00 | 99.70 | 8 |
| ATOM | 2165 | N | ALA | 338 | 68.357 | 237.780 | 103.283 | 1.00 | 100.00 | 7 |
| ATOM | 2166 | CA | ALA | 338 | 67.725 | 237.719 | 104.593 | 1.00 | 100.00 | 6 |
| ATOM | 2167 | CB | ALA | 338 | 66.491 | 236.815 | 104.538 | 1.00 | 97.78 | 6 |
| ATOM | 2168 | C | ALA | 338 | 68.757 | 237.183 | 105.613 | 1.00 | 100.00 | 6 |
| ATOM | 2169 | O | ALA | 338 | 68.666 | 237.476 | 106.808 | 1.00 | 100.00 | 8 |
| ATOM | 2170 | N | ALA | 339 | 69.760 | 236.444 | 105.118 | 1.00 | 100.00 | 7 |
| ATOM | 2171 | CA | ALA | 339 | 70.818 | 235.859 | 105.952 | 1.00 | 97.64 | 6 |
| ATOM | 2172 | CB | ALA | 339 | 71.768 | 235.026 | 105.099 | 1.00 | 92.80 | 6 |
| ATOM | 2173 | C | ALA | 339 | 71.601 | 236.886 | 106.762 | 1.00 | 96.62 | 6 |
| ATOM | 2174 | O | ALA | 339 | 71.297 | 237.116 | 107.934 | 1.00 | 94.32 | 8 |
| ATOM | 2175 | N | ALA | 340 | 72.601 | 237.505 | 106.136 | 1.00 | 97.27 | 7 |
| ATOM | 2176 | CA | ALA | 340 | 73.422 | 238.507 | 106.814 | 1.00 | 97.97 | 6 |
| ATOM | 2177 | CB | ALA | 340 | 74.502 | 239.049 | 105.873 | 1.00 | 92.72 | 6 |
| ATOM | 2178 | C | ALA | 340 | 72.579 | 239.652 | 107.376 | 1.00 | 97.43 | 6 |
| ATOM | 2179 | O | ALA | 340 | 72.806 | 240.094 | 108.500 | 1.00 | 95.64 | 8 |
| ATOM | 2180 | N | ALA | 341 | 71.575 | 240.083 | 106.615 | 1.00 | 97.30 | 7 |
| ATOM | 2181 | CA | ALA | 341 | 70.702 | 241.172 | 107.034 | 1.00 | 97.50 | 6 |
| ATOM | 2182 | CB | ALA | 341 | 69.729 | 241.525 | 105.933 | 1.00 | 96.32 | 6 |
| ATOM | 2183 | C | ALA | 341 | 69.951 | 240.870 | 108.319 | 1.00 | 100.00 | 6 |
| ATOM | 2184 | O | ALA | 341 | 70.023 | 241.647 | 109.274 | 1.00 | 100.00 | 8 |
| ATOM | 2185 | N | ALA | 342 | 69.232 | 239.750 | 108.348 | 1.00 | 100.00 | 7 |
| ATOM | 2186 | CA | ALA | 342 | 68.477 | 239.381 | 109.544 | 1.00 | 100.00 | 6 |
| ATOM | 2187 | CB | ALA | 342 | 67.555 | 238.199 | 109.266 | 1.00 | 100.00 | 6 |
| ATOM | 2188 | C | ALA | 342 | 69.417 | 239.066 | 110.701 | 1.00 | 99.76 | 6 |
| ATOM | 2189 | O | ALA | 342 | 69.171 | 239.485 | 111.831 | 1.00 | 99.57 | 8 |
| ATOM | 2190 | N | ALA | 343 | 70.513 | 238.369 | 110.404 | 1.00 | 98.98 | 7 |
| ATOM | 2191 | CA | ALA | 343 | 71.494 | 238.003 | 111.424 | 1.00 | 98.76 | 6 |
| ATOM | 2192 | CB | ALA | 343 | 72.595 | 237.124 | 110.816 | 1.00 | 94.09 | 6 |
| ATOM | 2193 | C | ALA | 343 | 72.098 | 239.265 | 112.033 | 1.00 | 98.91 | 6 |
| ATOM | 2194 | O | ALA | 343 | 72.278 | 239.361 | 113.254 | 1.00 | 96.27 | 8 |
| ATOM | 2195 | N | ALA | 344 | 72.350 | 240.252 | 111.173 | 1.00 | 99.66 | 7 |
| ATOM | 2196 | CA | ALA | 344 | 72.928 | 241.521 | 111.601 | 1.00 | 100.00 | 6 |
| ATOM | 2197 | CB | ALA | 344 | 73.314 | 242.383 | 110.391 | 1.00 | 98.13 | 6 |
| ATOM | 2198 | C | ALA | 344 | 71.964 | 242.262 | 112.514 | 1.00 | 98.63 | 6 |
| ATOM | 2199 | O | ALA | 344 | 72.365 | 243.165 | 113.249 | 1.00 | 99.84 | 8 |
| ATOM | 2200 | N | ALA | 345 | 70.696 | 241.866 | 112.478 | 1.00 | 97.69 | 7 |
| ATOM | 2201 | CA | ALA | 345 | 69.681 | 242.497 | 113.312 | 1.00 | 100.00 | 6 |
| ATOM | 2202 | CB | ALA | 345 | 68.315 | 242.474 | 112.605 | 1.00 | 96.25 | 6 |
| ATOM | 2203 | C | ALA | 345 | 69.596 | 241.795 | 114.674 | 1.00 | 100.00 | 6 |
| ATOM | 2204 | O | ALA | 345 | 69.655 | 242.445 | 115.725 | 1.00 | 98.58 | 8 |
| ATOM | 2205 | N | ALA | 346 | 69.530 | 240.462 | 114.623 | 1.00 | 100.00 | 7 |
| ATOM | 2206 | CA | ALA | 346 | 69.435 | 239.584 | 115.792 | 1.00 | 100.00 | 6 |
| ATOM | 2207 | CB | ALA | 346 | 70.153 | 238.270 | 115.523 | 1.00 | 100.00 | 6 |
| ATOM | 2208 | C | ALA | 346 | 69.883 | 240.165 | 117.123 | 1.00 | 99.45 | 6 |
| ATOM | 2209 | O | ALA | 346 | 69.069 | 240.324 | 118.033 | 1.00 | 100.00 | 8 |
| ATOM | 2210 | N | ALA | 347 | 71.166 | 240.496 | 117.236 | 1.00 | 99.03 | 7 |
| ATOM | 2211 | CA | ALA | 347 | 71.689 | 241.061 | 118.478 | 1.00 | 100.00 | 6 |
| ATOM | 2212 | CB | ALA | 347 | 71.898 | 239.963 | 119.520 | 1.00 | 99.73 | 6 |
| ATOM | 2213 | C | ALA | 347 | 72.978 | 241.848 | 118.278 | 1.00 | 100.00 | 6 |
| ATOM | 2214 | O | ALA | 347 | 73.959 | 241.337 | 117.729 | 1.00 | 100.00 | 8 |
| ATOM | 2215 | N | ALA | 348 | 72.954 | 243.096 | 118.737 | 1.00 | 100.00 | 7 |
| ATOM | 2216 | CA | ALA | 348 | 74.087 | 244.013 | 118.645 | 1.00 | 100.00 | 6 |
| ATOM | 2217 | CB | ALA | 348 | 74.381 | 244.373 | 117.180 | 1.00 | 100.00 | 6 |
| ATOM | 2218 | C | ALA | 348 | 73.737 | 245.283 | 119.431 | 1.00 | 100.00 | 6 |
| ATOM | 2219 | O | ALA | 348 | 72.850 | 246.053 | 119.040 | 1.00 | 99.90 | 8 |
| ATOM | 2220 | N | ALA | 349 | 74.443 | 245.457 | 120.533 | 1.00 | 100.00 | 7 |
| ATOM | 2221 | CA | ALA | 349 | 74.238 | 246.595 | 121.447 | 1.00 | 100.00 | 6 |
| ATOM | 2222 | CB | ALA | 349 | 75.383 | 246.674 | 122.455 | 1.00 | 100.00 | 6 |
| ATOM | 2223 | C | ALA | 349 | 74.172 | 247.921 | 120.677 | 1.00 | 100.00 | 6 |
| ATOM | 2224 | O | ALA | 349 | 73.527 | 248.885 | 121.113 | 1.00 | 100.00 | 8 |
| ATOM | 2225 | N | ALA | 350 | 74.845 | 247.951 | 119.544 | 1.00 | 99.04 | 7 |
| ATOM | 2226 | CA | ALA | 350 | 74.882 | 249.149 | 118.692 | 1.00 | 100.00 | 6 |
| ATOM | 2227 | C | ALA | 350 | 73.465 | 249.495 | 118.219 | 1.00 | 100.00 | 6 |
| ATOM | 2228 | O | ALA | 350 | 72.940 | 248.886 | 117.276 | 1.00 | 100.00 | 8 |
| ATOM | 2229 | CB | ALA | 350 | 75.775 | 248.902 | 117.475 | 1.00 | 100.00 | 6 |
| ATOM | 2230 | N | ALA | 351 | 72.884 | 250.468 | 118.902 | 1.00 | 100.00 | 7 |
| ATOM | 2231 | CA | ALA | 351 | 71.524 | 250.954 | 118.606 | 1.00 | 100.00 | 6 |
| ATOM | 2232 | CB | ALA | 351 | 70.519 | 250.326 | 119.573 | 1.00 | 98.63 | 6 |
| ATOM | 2233 | C | ALA | 351 | 71.472 | 252.478 | 118.744 | 1.00 | 100.00 | 6 |
| ATOM | 2234 | O | ALA | 351 | 71.050 | 253.128 | 117.767 | 1.00 | 100.00 | 8 |
| ATOM | 2235 | OT | ALA | 351 | 71.875 | 253.007 | 119.807 | 1.00 | 98.22 | 8 |
| ATOM | 2236 | P3 | ANP | 100 | 87.742 | 257.555 | 114.645 | 1.00 | 42.42 | 15 |
| ATOM | 2237 | O1G | ANP | 1001 | 87.10 | 257.082 | 115.999 | 1.00 | 54.42 | 8 |
| ATOM | 2238 | O2G | ANP | 1001 | 87.349 | 256.642 | 113.476 | 1.00 | 23.41 | 8 |
| ATOM | 2239 | O3G | ANP | 1001 | 89.289 | 257.514 | 114.872 | 1.00 | 37.25 | 8 |
| ATOM | 2240 | P2 | ANP | 1001 | 87.963 | 260.430 | 114.225 | 1.00 | 68.82 | 15 |
| ATOM | 2241 | O1B | ANP | 1001 | 87.627 | 261.465 | 115.351 | 1.00 | 59.81 | 8 |
| ATOM | 2242 | O2B | ANP | 1001 | 89.496 | 260.281 | 114.015 | 1.00 | 69.69 | 8 |
| ATOM | 2243 | N3B | ANP | 1001 | 87.264 | 259.008 | 114.382 | 1.00 | 53.72 | 7 |
| ATOM | 2244 | P1 | ANP | 1001 | 86.413 | 260.097 | 112.093 | 1.00 | 52.56 | 15 |
| ATOM | 2245 | O1A | ANP | 1001 | 86.439 | 260.381 | 110.627 | 1.00 | 59.00 | 8 |
| ATOM | 2246 | O2A | ANP | 1001 | 86.900 | 258.693 | 111.952 | 1.00 | 53.10 | 8 |
| ATOM | 2247 | O3A | ANP | 1001 | 87.279 | 261.039 | 112.972 | 1.00 | 53.02 | 8 |
| ATOM | 2248 | O5′ | ANP | 1001 | 84.992 | 260.144 | 112.683 | 1.00 | 49.93 | 8 |
| ATOM | 2249 | C5′ | ANP | 1001 | 84.097 | 259.019 | 112.514 | 1.00 | 49.91 | 6 |
| ATOM | 2250 | C4′ | ANP | 1001 | 82.627 | 259.436 | 112.572 | 1.00 | 41.87 | 6 |
| ATOM | 2251 | O4′ | ANP | 1001 | 82.464 | 260.819 | 112.270 | 1.00 | 38.43 | 8 |
| ATOM | 2252 | C3′ | ANP | 1001 | 81.812 | 258.632 | 111.534 | 1.00 | 50.98 | 6 |
| ATOM | 2253 | O3′ | ANP | 1001 | 81.126 | 257.509 | 112.115 | 1.00 | 59.84 | 8 |
| ATOM | 2254 | C2′ | ANP | 1001 | 80.815 | 259.637 | 110.975 | 1.00 | 50.96 | 6 |
| ATOM | 2255 | O2′ | ANP | 1001 | 79.583 | 259.607 | 111.738 | 1.00 | 59.86 | 8 |
| ATOM | 2256 | C1′ | ANP | 1001 | 81.593 | 260.934 | 111.143 | 1.00 | 49.40 | 6 |
| ATOM | 2257 | N9 | ANP | 1001 | 82.268 | 261.368 | 109.931 | 1.00 | 45.89 | 7 |
| ATOM | 2258 | C8 | ANP | 1001 | 83.614 | 261.549 | 109.862 | 1.00 | 49.56 | 6 |
| ATOM | 2259 | N7 | ANP | 1001 | 83.939 | 262.006 | 108.632 | 1.00 | 47.99 | 7 |
| ATOM | 2260 | C5 | ANP | 1001 | 82.788 | 262.114 | 107.889 | 1.00 | 42.44 | 6 |
| ATOM | 2261 | C6 | ANP | 1001 | 82.433 | 262.508 | 106.593 | 1.00 | 42.99 | 6 |
| ATOM | 2262 | N6 | ANP | 1001 | 83.400 | 262.938 | 105.676 | 1.00 | 36.10 | 7 |
| ATOM | 2263 | N1 | ANP | 1001 | 81.137 | 262.471 | 106.213 | 1.00 | 41.43 | 7 |
| ATOM | 2264 | C2 | ANP | 1001 | 80.158 | 262.075 | 107.018 | 1.00 | 42.75 | 6 |
| ATOM | 2265 | N3 | ANP | 1001 | 80.331 | 261.673 | 108.272 | 1.00 | 42.93 | 7 |
| ATOM | 2266 | C4 | ANP | 1001 | 81.657 | 261.692 | 108.718 | 1.00 | 42.82 | 6 |

Once a dataset such as the one in Table 2 is collected, the information is used to determine the three-dimensional structure of the molecule in the crystal. However, in the absence alone of a suitable molecular model, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal. (Blundel et al., 1976, Protein Crystallography, Academic Press).

Another method of obtaining phase information is by molecular replacement, which is a method of calculating initial phases for a new crystal of a polypeptide or polypeptide co-complex whose structure coordinates are unknown by orienting and positioning a related polypeptide whose structure coordinates are known within the unit cell of the new crystal so as to best account for the observed diffraction pattern of the new crystal. To enable this, the related molecule must have a similar three dimensional structure. Briefly, the principle behind the method of molecular replacement is as follows. A suitable search model, whose three-dimensional structure is similar to that of the unknown target, is identified first. The search model is then rotated and translated within the unit cell of the unknown. For each position of the model, a set of structure factors of the model is computed. These calculated structure factors are then compared with the measured intensities of the unknown and expressed as correlation coefficients. The solution with the highest correlation coefficient is selected as the true solution. These concepts are discussed at length in the book “The Molecular Replacement Method edited by Rossmann (1972, Int. Sci. Rev. Ser. No 13, Gordon & Breach, New York).

A third method of phase determination is multi-wavelength anomalous dispersion or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

A fourth method of determining phase information is single wavelength anomalous w dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. A detailed discussion of SAD analysis can be found in Brodersen et al., 2000, Acta Cryst., D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

The MK-2 structure was determined using the method of molecular replacement. Initially, a homology model of MK-2 was constructed using the crystal structures of calcium calmodulin-dependent protein kinase (36% identical at the level of amino acid sequence, 1A06), phosphorylase kinase (30%, 2PHK) and cyclic AMP-dependent protein kinase (29%, 1ATP). This resulted in a model that consisted of residues 64-327 for the minimal kinase domain of MK-2. Residues 64-142 were assigned to be part of the N-terminal lobe of MK-2 and residues 143-327 were designated as the C-terminal domain.

The homology model was then used as the search model for molecular replacement using several program suites including X-PLOR, AMORE and EPMR. In order to arrive at a consistent solution, molecular replacement calculations were repeated by varying several of the parameters including: resolution of the data, Patterson vector length, B-factor of the model, the number of molecules per asymmetric unit and space group (F432 or F4₁32). The high symmetry of the crystal lattice (face-centered cubic lattice) and the relatively high solvent content of the crystals (72%) presented significant technical challenges to the molecular replacement calculations. Of the three program suites used, only program EPMR (Kissinger C R, Gehlhaar D R and Fogel D B Acta Crystallogr (1999) D55, 484-491) was successful in arriving at a consistent and reasonable solution to the three rotation and three translation variables. Better results were obtained with a poly-alanine template of the homology model where all the non-glycine amino acids were truncated back to alanine.

For the successful molecular replacement calculation replacement calculation using EPMR, diffraction data in the resojution range 15-4 angstroms was used. The top solution had a correlation coefficient of 0.522 and an R-factor of 54.2%. The peak height of the top solution was 14.2 sigma where sigma is the root mean square fluctuation in the correlation function between Fobs and Fcalc. The rotation and translation parameters for the top solution are listed below for the two domains of MK-2.

| Domain | Alpha | Beta | Gamma | X | Y | Z |
|---|---|---|---|---|---|---|
| N-term | 187.60 | 153.98 | 96.51 | 88.88 | 251.12 | 108.02 |
| C-term | 172.99 | 151.99 | 81.30 | 88.17 | 250.39 | 108.21 |

The N- and C-terminal domains of MK-2 homology were rotated approximately 11 degrees relative to those in the homology model.

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically realistic conformation that fits the map precisely.

After a model is generated, the structure is refined. Refinement is the process of minimizing the function Φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function Φ converges on a minimum wherein the model fits the electron density map and is stereochemically and conformationally reasonable. During refinement, ordered solvent molecules are added to the structure. The transformed coordinates of the MK-2 homology model were used as the initial model for crystallographic refinement. A number of different crystallographic refinement protocols were evaluated. The best result was obtained with a dynamic torsion angle refinement procedure where the model was assigned an initial temperature of 2500 Kelvin. The R-factor and the Rfree at the end of refinement were 24.7% and 30.7% respectively.

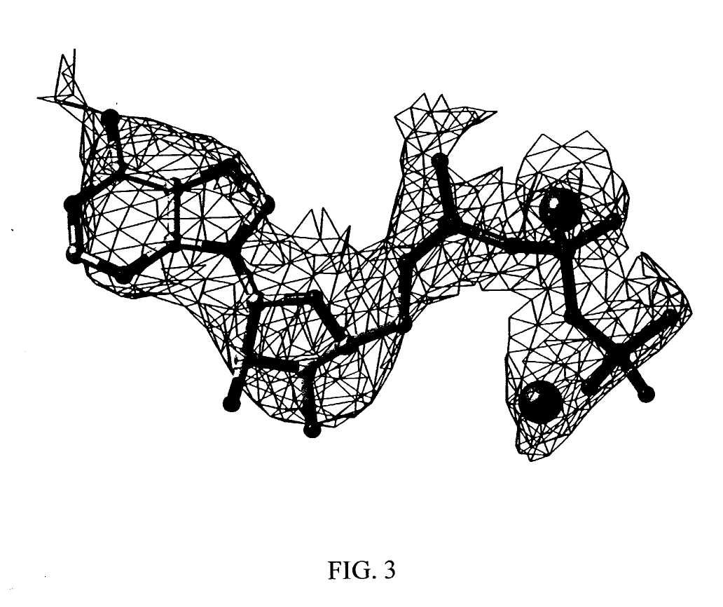

With the best solution from the molecular replacement calculations, an initial model of MK-2 was constructed using the homology model. This model was then subjected to several rounds of crystallographic refinement. An electron density map was then calculated. Well-defined electron density was visible for AMP-PNP at the ATP site of MK-2 as shown in FIG. 3.

Well-connected electron density was also observed for the glycine flap region (residues 71-76), presumably due to strong interactions with AMP-PNP. In addition, electron density was present for several of the missing amino acid residues in some of the loops that were excluded from the homology model.

In an iterative fashion as described before, the MK-2 model was gradually improved by including more atoms into the structure. The N-terminus was extended all the way to residue 45, the fist amino acid residue of MK-2 construct that was used for crystallization. Similarly, the C-terminus was extended to residue 351. This includes part of the auto-inhibitory domain of MK-2. The R-factor and Rfree at the end of final refinement were 24.7% and 30.7% (8.0-3.0 A resolution) respectively. The following amino acids have been excluded from the current model since they could not be clearly located in the electron density: 156-157, 216-226, 268-274 and 352-371. In addition, the following residues have been modeled as alanine since their side chains could not be identified unambiguously: Arg 153, Asp 155, Lys 197, Met 275, Lys 276, Ile 277, Arg 278, Glu 309, Pro 310, Thr 311, Gln 312, Ser 328, Thr 329, Lys 330, Val 331, Pro 332, Gly 333, Thr 334, Pro 335, Leu 336, His 337, Thr 338, Ser 339, Arg 340, Val 341, Leu 342, Lys 343, Glu 344, Asp 345, Lys 346, Glu 347, Arg 348, Trp 349, Glu 350 and Asp 351.

The ATP-analogue, AMP-PNP, binds in a narrow pocket at the ATP site of MK-2. The ATP binding site is defined by amino acid residues (within a radius of 8.0 A around AMP-PNP): 69-80, 90-95, 104, 108, 118-119, 136-147, 184-195, 204-210, and 224-226. Specifically, amino acid residues 69-80 are: Val 69, Leu 70, Gly 71, Leu 72, Gly 73, Ile 74, Asn 75, Gly 76, Lys 77, Val 78, Leu 79, and Gln 80. Amino acid residues 90-95 are: Phe 90, Ala 91, Leu 92, Lys 93, Met 94, and Leu 95. Amino acid residues 104 and 108 are Glu 104 and His 108 and amino acid residues 118-119 are Val 118 and Arg 119. The segment 136-147 contains the following amino acids: Ile 136, Val 137, Met 138, Glu 138, Cys 140, Leu 141, Asp 142, Gly 143, Gly 144, Glu 145, Leu 146, and Phe 147. The peptide segment 184-195 consists of the amino acids: His 184, Arg 185, Asp 186, Val 187, Lys 188, Pro 189, Glu 190, Asn 191, Leu 192, Leu 193, Tyr 194, and Thr 195. Amino acid residues 204-210 are: Lys 204, Leu 205, Thr 206, Asp 207, Phe 208, Gly 209, and Phe 210.

The adenine ring of AMP-PNP forms hydrogen bonding interactions with the peptide backbone of residues Glu 139 and Leu 141. In addition, the bicyclic ring of adenine forms close contacts with residues Ala 91, Met 138, Cys 140, Val 118, Leu 70, and Val 78. The ribose sugar of AMP-PNP interacts with residues, Gly 71, Leu 72, Glu 145, and Leu 193. The triphosphate moiety is surrounded by amino acid residues, Leu 72, Gly 73, Ile 74, Asn 75, Val 78, Asp 207, Lys 93, Lys 188, Asn 191, Glu 190, and Thr 206. The auto-inhibitory domain of MK-2 folds back on the protein and approaches the binding sites for ATP and the peptide substrate. As a result, the ATP binding site is constricted even further.

The atomic structure coordinates and machine readable media of the invention have a variety of uses. The present invention encompasses the structure coordinates and other information, e.g., amino acid sequence, connectivity tables, vector-based representations, temperature factors, etc., used to generate the three-dimensional structures of the polypeptides for use in the software programs described below and other software programs. For example, the coordinates listed in Table 3 are useful for solving the three-dimensional crystal or solution structures of other proteins to high resolution. MK-2 can be crystallized in a diffraction lattice of other homologous proteins.

Additionally, the invention encompasses machine readable media embedded with the three-dimensional structures of the models described herein, or with portions thereof. As used herein, “machine readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an Optical Character Recognition (OCR).

A variety of data storage structures are available to a skilled artisan for creating a computer readable medium having recorded thereon the atomic structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics; http://www.rcsb.org/pdb/docs/format/pdbguide2.2/guide2.2_frame.html); Cambridge Crystallographic Data Centre format (http://www.ccdc.cam.ac.uk/support/csd_doc/volume3/z323.html); Structure-data (“SD”) file format (MDL Information Systems, Inc.; Dalby et al., 1992, J. Chem. Inf. Comp. Sci. 32:244-255), and line-notation, e.g., as used in SMILES (Weininger, 1988, J. Chem. Inf. Comp. Sci. 28:31-36). Methods of converting between various formats read by different computer software will be readily apparent to those of skill in the art, e.g., BABEL (v. 1.06, Walters & Stahl, © 1992, 1993, 1994; http://www.brunel.ac.uk/departments/chem/babel.htm.) All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

While Cartesian coordinates are important and convenient representations of the three-dimensional structure of a polypeptide, those of skill in the art will readily recognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein or protein co-complex in which an atom is located, and connectivity information, which indicates to which atoms a particular atom is bonded, is also useful for representing a three-dimensional molecular structure.

Uses of the Atomic Structure Coordinates: Drug Design

Structural information, often in the form of atomic structure coordinates, may also be used in a variety of molecular modeling and computer-based screening applications to, for example, design variants that have altered biological properties or to computationally design, screen for and/or identify compounds that bind to the MK-2 protein or to fragments of the MK-2 protein. Such compounds may be used as lead compounds in pharmaceutical efforts to identify compounds that may be useful as drugs in the treatment of inflammatory diseases or inflammation.

Thus, in a further aspect of the invention, the data from the crystal structure of MK-2 is used to evaluate compounds for their utility as drugs. These methods comprise designing and synthesizing candidate compounds using the atomic coordinates of the three dimensional structure of such co-crystals and screening for its utility in various pharmaceutical applications. Examples of such pharmaceutical applications include the treatment of inflammation, inflammatory disease states, and related conditions.

In one embodiment, the co-crystals and structure coordinates obtained therefrom are useful for identifying and/or designing compounds that inhibit MK-2 as an approach towards developing new therapeutic agents for inflammation and inflammatory disease states. For example, a high resolution X-ray structure will often show the locations of ordered solvent molecules around the protein, and in particular at or near putative binding sites on the protein. This information can then be used to design molecules that bind at these sites, which then could be synthesized and tested for binding in biological assays. (Travis, 1993, Science 262:1374)

In another embodiment, the structures are probed with a plurality of molecules to determine their ability to bind to the MK-2 protein at various sites. Such compounds can be used as targets or leads in medicinal chemistry efforts to identify, for example, inhibitors of potential therapeutic importance in the treatment of inflammation, inflammatory disease states or other disorders.

In specific embodiments described herein, the high resolution X-ray structures of the MK-2 co-complex show details of the interactions between MK-2 and AMP-PNP. This information can be used to design molecules that bind to the sites of interaction, thereby blocking co-complex formation.

In yet another embodiment, the structures can be used to computationally screen small molecule databases for chemical entities or compounds that can bind in whole, or in part, to MK-2. In this screening, the quality of fit of such entities or compounds to the binding site may be judged either by shape complementarity or by estimated interaction energy. (Meng et al., 1992, J. Comp. Chem. 13:505-524).

The design of compounds that bind to MK-2 according to this invention generally involves consideration of two factors. First, the compound must be capable of physically and structurally associating with MK-2. This association can be covalent or non-covalent. For example, covalent interactions may be important for designing suicide or irreversible inhibitors of a protein. Non-covalent molecular interactions important in the association of MK-2 include hydrogen bonding, ionic and other polar interactions, interactions as well as van der Waals interactions. Second, the compound must be able to assume a conformation that allows it to associate with the MK-2 protein. Although certain portions of the compound will not directly participate in this association with the protein, those portions may still influence the overall conformation of the molecule. This, in turn, may have a significant impact on potency. Such conformational requirements include the overall three-dimensional structure and orientation of the chemical group or compound in relation to all or a portion of the binding site, or the spacing between functional groups of a compound comprising several chemical groups that directly interact with the protein.

The potential inhibitory or binding effect of a chemical compound on MK-2 may be analyzed prior to its actual synthesis and testing by the use of computer modeling techniques. If the theoretical structure of the given compound suggests insufficient interaction and association between it and the protein, synthesis and testing of the compound is unnecessary. However, if computer modeling indicates a strong interaction, the molecule may then be synthesized and tested for its ability to bind to the protein and inhibit its activity. In this manner, synthesis of ineffective compounds may be avoided.

An inhibitory or other binding compound of MK-2 may be computationally evaluated and designed by means of a series of steps in which chemical groups or fragments are screened and selected for their ability to associate with the individual binding pockets or interface surfaces of each of the proteins. One skilled in the art may use one of several methods to screen chemical groups or fragments for their ability to associate with MK-2. This process may begin by visual inspection of, for example, the protein/protein interfaces or the various binding sites of MK-2 on the computer screen based on the MK-2, AMP-PNP, magnesium, and SC-83598 co-complex coordinates. Selected fragments or chemical groups may then be positioned in a variety of orientations, or docked, at an individual surface of MK-2 that participates in a protein/protein interface in the co-complex or in other binding sites of MK-2. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical groups. These include:

1. GRID (Goodford, 1985, J. Med. Chem. 28:849-857). GRID is available from Oxford University, Oxford, UK;

2. MCSS (Miranker & Karplus, 1991, Proteins: Structure, Function and Genetics 11:29-34). MCSS is available from Molecular Simulations, Burlington, Mass.;

3. AUTODOCK (Goodsell & Olsen, 1990, Proteins: Structure, Function, and Genetics 8:195-202). AUTODOCK is available from Scripps Research Institute, La Jolla, Calif.;

4. DOCK (Kuntz et al., 1982, J. Mol. Biol. 161:269-288). DOCK is available from University of California, San Francisco, Calif.;

5. FlexE (Clausen H, Buning C, Rarey M and Lengauer T) J. Mol. Biol. (2001) 308, 377-395. FlexE is available from Tripos, St. Louis, Mo.;

6. Glide, Glide is available from Schrodinger, Portland, Oreg.;

7. Gold, Jones et al. J. Mol. Biol. 245, 43-53, 1995;

8. QXP, McMartin C, Bohacek RS. J Comput Aided Mol Des 1997 11:333-44;

9. ICM. (http://www.molsoft.com). Available from Molsoft, San Diego, Calif.; and

10. FlexX. [Sybl, Tripos, St. Louis, Mo.].

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of MK-2. This would be followed by manual model building using software such as QUANTA or SYBYL.

Useful programs to aid one of skill in the art in connecting the individual chemical groups or fragments include:

1. CAVEAT (Bartlett et al., 1989, ‘CAVEAT: A Program to Facilitate the Structure-Derived Design of Biologically Active Molecules’. In Molecular Recognition in Chemical and Biological Problems', Special Pub., Royal Chem. Soc. 78:182-196). CAVEAT is available from the University of California, Berkeley, Calif.;

2. 3D Database systems such as MACCS-3D (MDL Information Systems, San Leandro, Calif.). This area is reviewed in Martin, 1992, J. Med. Chem. 35:2145-2154); and

3. HOOK (available from Molecular Simulations, Burlington, Mass.).

Instead of proceeding to build an inhibitor of MK-2 in a step-wise fashion one fragment or chemical group at a time, as described above, MK-2-binding compounds or inhibitors may be designed as a whole or ‘de novo’ using either an empty binding site or the surface of a protein that participates in protein/protein interactions in a co-complex, or optionally including some portion(s) of a known inhibitor(s). These methods include:

1. LUDI (Bohm, 1992, J. Comp. Aid. Molec. Design 6:61-78). LUDI is available from Molecular Simulations, Inc., San Diego, Calif.;

2. LEGEND (Nishibata & Itai, 1991, Tetrahedron 47:8985). LEGEND is available from Molecular Simulations, Burlington, Mass.; and

3. LeapFrog (available from Tripos, Inc., St. Louis, Mo.).

Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210.

Once a compound has been designed or selected by the above methods, the efficiency with which that compound may bind to MK-2 may be tested and optimized by computational evaluation. An effective inhibitor of MK-2 must preferably demonstrate a relatively small difference in energy between its bound and free states (i.e., it must have a small deformation energy of binding). Thus, the most efficient inhibitors should preferably be designed with a deformation energy of binding of not greater than about 10 kcal/mol, preferably, not greater than 7 kcal/mol. Inhibitors may interact with the protein in more than one conformation that is similar in overall binding energy. In those cases, the deformation energy of binding is taken to be the difference between the energy of the free compound and the average energy of the conformations observed when the inhibitor binds to the protein.

A compound selected or designed for binding to or inhibiting MK-2 may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the protein when the inhibitor is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©p1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

The computer-assisted methods for designing an inhibitor of MK-2 activity can be de novo or based on a candidate compound. An example of a computer-assisted method for designing an inhibitor of MK-2 activity de novo would thus involve the steps of: (1) supplying a computer modeling application with a set of structure coordinates of a molecule or molecular complex comprising at least a portion of an MK-2 or MK-2-like ATP binding site, the ATP binding site comprising the 69-80, 90-95, 104, 108, 118-119, 136-147, 184-195, 204-210, and 224-226 amino acid sequence; (2) computationally building a chemical entity represented by a set of structure coordinates; and (3) determining whether the chemical entity is an inhibitor expected to bind to or interfere with the molecule or molecular complex, wherein binding to or interfering with the molecule or molecular complex is indicative of potential inhibition of MK-2 activity.

An example of a computer-assisted method for designing an inhibitor of MK-2 activity based on a candidate compound would involve the steps of (1) supplying a computer modeling application with a set of structure coordinates of a molecule or molecular complex comprising at least a portion of an MK-2 or MK-2-like ATP binding site, the ATP binding site comprising the 69-80, 90-95, 104, 108, 118-119, 136-147, 184-195,204-210, and224-226 amino acid sequence; (2) supplying the computer modeling application with a set of structure coordinates of a chemical entity; (3) evaluating the potential binding interactions between the chemical entity and ATP binding site of the molecule or molecular complex; (4) structurally modifying the chemical entity to yield a set of structure coordinates for a modified chemical entity; and (5) determining whether the modified chemical entity is an inhibitor.

Once an inhibitor or MK-2 binding compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to MK-2 by the same computer methods described in detail above.

An example of such a computer-assisted method for identifying an inhibitor of MK-2 activity would thus involve (1) supplying a computer modeling application with a set of structure coordinates of a molecule or molecular complex comprising at least a portion of an MK-2 or MK-2-like compound, (2) supplying the computer modeling application with a set of structure coordinates of a chemical entity; and (3) determining whether the chemical entity is an inhibitor expected to bind to or interfere with the molecule or molecular complex.

The structure coordinates of the MK-2 co-complex, or of MK-2 alone, or of portions thereof, are particularly useful to solve the structure of other co-complexes of MK-2, of mutants, of the MK-2 co-complex further complexed to another molecule, or of the crystalline form of any other protein or protein co-complex with significant amino acid sequence homology to any functional domain of MK-2.

One method that may be employed for this purpose is molecular replacement. In this method, the unknown co-crystal structure, whether it is another MK-2 co-complex, a mutant, a MK-2 co-complex that is further complexed to another molecule, or the crystal of some other protein or protein co-complex with significant amino acid sequence homology to any functional domain of one of the proteins in the co-complex crystal, may be determined using phase information from the present MK-2 co-complex structure coordinates. This method will provide an accurate three-dimensional structure for the unknown protein or protein co-complex in the new crystal more quickly and efficiently than attempting to determine such information ab initio.

If an unknown crystal form has the same space group as and similar cell dimensions to the known co-complex crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. However, if the space groups and/or cell dimensions of the two crystal forms are different, then this approach will not work and molecular replacement must be used in order to derive phases for the unknown crystal form.

The techniques of X-ray diffraction can be employed in the study of the co-complexes of MK-2. This information may thus be used to optimize known inhibitors of MK-2 and more importantly, to design and synthesize novel classes of inhibitors of MK-2.

Subsets of the atomic structure coordinates can also be used in any of the above methods. Particularly useful subsets of the coordinates include, but are not limited to, coordinates of single domains, coordinates of residues lining an active site, coordinates of residues that participate in important protein-protein contacts at an interface, and Cα coordinates. For example, the coordinates of one domain of a protein that contains the active site may be used to design inhibitors that bind to that site, even though the protein is fully described by a larger set of atomic coordinates. Therefore, as described in detail for the specific embodiments, below, a set of atomic coordinates that define the entire polypeptide chain, although useful for many applications, do not necessarily need to be used for the methods described herein.

Uses of Subsets of Atomic Coordinates in Specific Embodiments

The structure coordinates of the present invention, and subsets thereof, are useful for designing or screening for compounds that bind to the MK-2 protein. The high resolution X-ray structure of the co-complexes of the present invention show details of the interactions between MK-2 and AMP-PNP. This information can be used to design and/or screen for compounds that act as inhibitors of MK-2, thereby inhibiting the biosynthesis of TNF-α at a post-transcriptional level.

Those of skill in the art will recognize that the complete set of MK-2 co-complex structure coordinates will be useful in the methods of the present invention. Those of skill in the art will further recognize that the coordinates of the MK-2 co-complex will be useful separate from the coordinates of the MK-2 protein. In addition, those of skill in the art will recognize that subsets of the structure coordinates of the MK-2 protein, such as the coordinates of a single domain or interface or binding pocket, will be useful in the methods of the invention, as discussed in more detail below.

Using the techniques for solving the structure of MK-2 described above, it has been determined that the ATP-analogue binds in a narrow pocket at the ATP site of MK-2. The ATP binding site is defined by amino acid residues (within a radius of 8.0A around AMP-PNP/Mg²⁺): 69-80, 90-95, 104, 108, 118-119, 136-147, 184-195, 204-210, and 224-226. Thus, the MK-2 coordinates, or a subset of the MK-2 coordinates at the ATP site of MK-2, are useful for designing and/or screening for compounds that disrupt the binding at the ATP site of MK-2. Such a compound could potentially be useful in disrupting the binding of ATP, ATP analogues, or other unrelated ligands to MK-2. A subset of MK-2 coordinates useful for this embodiment of the invention include those of amino acid residues 69-80, 90-95, 104, 108, 118-119, 136-147, 184-195, 204-210, and 224-226.

The adenine ring of AMP-PNP forms hydrogen bonding interactions with the peptide backbone of residues Glu 139 and Leu 141. In addition, the bicyclic ring of adenine forms close contacts with residues, Ala 91, Met 138, Cys 140, Val 118, Leu 70, and Val 78. The ribose sugar of AMP-PNP interacts with residues, Gly 71, Leu 72, Glu 145, and Leu 193. The triphosphate moiety is surrounded by amino acid residues, Leu 72, Gly 73, Ile 74, Asn 75, Val 78, Asp 207, Lys 93, Lys 188, Asn 191, Glu 190 and Thr 206. To the extent that these physical interactions assist in the formation or stabilization of the MK-2 co-complex, the MK-2 coordinates, or a subset of the MK-2 coordinates at these sites of MK-2, are useful for designing and/or screening for compounds that disrupt the stabilization and consequently possibly the formation of co-complexes of MK-2 and ATP analogues. A subset of MK-2 coordinates useful for this embodiment of the invention as it relates to the hydrogen bonding interactions with the adenine ring of AMP-PNP include those of amino acid residues Glu 139 and Leu 141. A subset of MK-2 coordinates useful for this embodiment of the invention as it relates to the contacts formed by the bicyclic ring of adenine include those of Ala 91, Met 138, Cys 140, Val 118, Leu 70, and Val 78. A subset of MK-2 coordinates useful for this embodiment of the invention as it relates to interactions with the ribose sugar of AMP-PNP include those of amino acid residues Gly 71, Leu 72, Glu 145, and Leu 193. A subset of MK-2coordinates useful for this embodiment of the invention as it relates to interactions with the triphosphate moiety include those of Leu 72, Gly 73, Ile 74, Asn 75, Val 78, Asp 207, Lys 93, Lys 188, Asn 191, Glu 190 and Thr 206.

The following examples illustrate the invention, but are not to be taken as limiting the various aspects of the invention so illustrated.

### EXAMPLES

### Example 1

Generation of MK-2 Protein

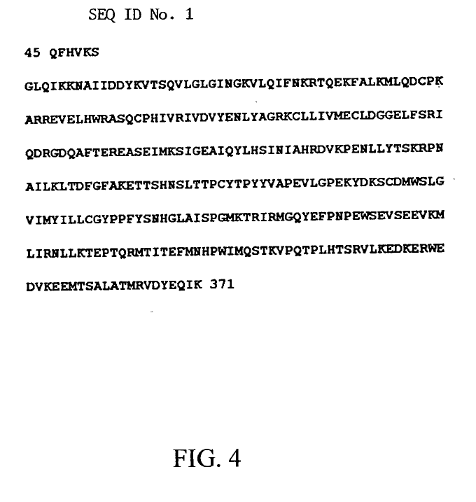

The specific MK-2 sequence (listed in FIG. 4) was used as a fusion protein with glutathiones transferase (GST) for expression in E-coli.

### Example 2

Protein Purification

Human MK-2 (45-371) was expressed as a GST fusion protein in E. coli BL21(LysS) cells. 500 g E. coli cell paste was suspended into 2L PBS and sonnicated using a microfluidizer under 10,000 psi pressure. The lysate was centrifuged twice at 11,300×g and the supernatant was collected each time. The supernatant was bound with 100 ml 50% PBS washed glutathione resin for 45 min at 4-8° C. The resin was washed with 10 column volumes of PBS with 1% Triton X-100, then 20 column volumes of PBS. To cleave the GST-tag, the resin was then mixed with 2500 Units of thrombin protease for 4 hours at room temperature. PMSF, DTT and glycerol were then added. The eluate was buffer exchanged against 40× its volume of dialysis buffer (50 mM Tris, pH 8.8, 2mM DTT, 5% glycerol). The dialyzed material was run over a MonoQ column using a 0-25 mM NaCl gradient over 20 column volumes (buffer A: 50 mM Tris, pH 8.8, 2 mM DTT, 5% glycerol; buffer B: same as buffer A except with 1 M NaCl). The most pure MK-2 (>98%) eluted at the front peak of ˜15 mM NaCl. This material was concentrated to 1-15 mg/mL in a Centricon protein concentrator or (MWCO=10 kD) and used in crystallization experiments.

### Example 3

Crystallization of MK-2

Crystals of MK-2(45-371) were grown by the sitting drop method of vapor diffusion at room temperature. A protein solution consisting of 1.5-15 mg/mL MK-2(45-371) in 50 mM Tris, pH 8.5-8.8, or 50 mM MES pH 6-6.3, 15 mM NaCl, 2 mM DTT, and 5% glycerol was mixed in a 1:1 ratio with a reservoir solution containing 1.6-2.6M ammonium sulfate and 100 mM sodium acetate, pH 4.2-5.4, or citrate pH 3.8-6.2. Small bipyramidal or prism-shaped crystals appeared in the drops in 1-2 days and grew to as large as 0.4 mm×0.4 mm over 1-3 weeks. The crystal structure was solved using crystals of MK-2 grown in the presence of a non-hydrolysable ATP analog (AMP-PNP), a 13-mer inhibitor peptide (SC-83598) and MgCl₂. This ternary complex was formed using enzyme/peptide/Mg²⁺/AMP-PNP molar ratios of 1:3:5:20, in a manner similar to that used in crystallizing a ternary complex of c-AMP-dependent protein kinase, as described by Zheng et al. in Crystal Structure of the Catalytic Subunit of cAMP-Dependent Protein Kinase Complexed with MgATP and Peptide Inhibitor, Biochemistry, 1993, Vol.32, No. 9, pages 2154-2161, 2155. Initial crystals of the MK-2 complex diffracted typically to 4-5 Angstroms at the Advanced Photon Source at Argonne National Laboratory. Crystallization additive screen kits from Hampton Research were employed to identify additives which, when added to the aforementioned crystallization conditions, improved the diffraction of the resulting crystals to 3.0 Angstroms.

### Example 4

MK-2 Structure Determination

Square-bipyramidal crystals of MK-2 were obtained as described in Example 2. These crystals belong to the space group F4₁32 (Space group No. 210) with face-centered cubic lattice and contain a single copy of the ternary complex in the asymmetric unit. The unit cell parameters are about 254.8 Angstroms along the three edges. The presence of additive improved the diffraction resolution to 3.0 Angstroms. Complete diffraction data has been obtained from several crystals of the putative ternary complex using synchrotron X-rays of Beamline 17ID at the Advanced Photon Source of the Argonne National Lab in Darian, Ill. The crystals were flash frozen in liquid nitrogen. A summary of the data set from individual crystals appears in Table 2, reproduced below.

TABLE 2
Summary of Diffraction Data From MK-2 Crystals

| Data Set | Crystal (1) | Crystal (2) | Crystal (3) |
|---|---|---|---|
| Resolution (A) | 40.0 − 3.0 | 40.0 − 3.3 | 40.0 − 3.3 |
| Rsymm | 7.7 | 8.6 | 7.6 |
| Completeness | 94.4 | 97.4 | 96.6 |
| Redundancy | 6.0 | 7.1 | 4.8 |
| Cell edge (A) | 254.0 | 253.5 | 253.5 |

Merging of diffraction data from different crystals results in a complete data set (99.8% complete with an overall R merge of 5.6%).

A homology model of MK-2 was constructed using the structures of cyclic-AMP dependent protein kinase (1ATP), the calmodulin-dependent protein kinase (1Ao6) and the phosphorylase kinase (2PHK). This resulted in a model of MK-2 that comprised of residues of 64-327 for the minimal kinase domain. The homology model was used as a search model for molecular replacement using the program EPMR. Better results were obtained with a poly-alanine template of the homology model where all the non-glycine amino acids were truncated back to alanine. Residues 64-142 were assigned to be part of the N-terminal lobe of MK-2 and residues 143-327 were designated as the C-terminal domain. Diffraction data in the resolution range 15-4.0 A were used for the molecular replacement calculations. The top solution had a correlation coefficient of 0.522 and an R-factor of 54.2%. The peak height of the top solution was 14.2 sigma where sigma is the root mean square fluctuation in the correlation function between Fobs and Fcalc. The rotation and translation parameters for the top solution are listed below for the two domains of MK-2.

| Domain | Alpha | Beta | Gamma | X | Y | Z |
|---|---|---|---|---|---|---|
| N-term | 187.60 | 153.98 | 96.51 | 88.88 | 251.12 | 108.02 |
| C-term | 172.99 | 151.99 | 81.30 | 88.17 | 250.39 | 108.21 |

The N- and C-terminal domains of MK-2 homology were rotated approximately 11 degrees relative to those in the homology model. The transformed coordinates of the MK-2 homology model were used as the initial model for crystallographic refinement. A number of different crystallographic refinement protocols were evaluated. The best result was obtained with a dynamic torsion and refinement procedure where the model was assigned an initial temperature of 2500 Kelvin. The R-factor and the R-free at the end of refinement were 24.7% and 30.7% respectively. An overall structure of MK-2 with the ligand, AMP-PNP, is shown in FIG. 1.

An electron density map was calculated at this stage. Well-defined electron density is visible for AMP-PNP and Mg²⁺ at the ATP site of MK-2 as shown in FIG. 3. In addition, electron density is visible for missing amino acids in some of the loop regions that were excluded from the initial model.

The ATP-analogue binds in a narrow pocket at the ATP site of MK-2. The ATP binding site is defined by amino acid residues (within a radius of 8.0A around AMP-PNP/Mg²⁺): 69-80, 90-95, 104, 108, 118-119, 136-147, 184-195, 204-210, and 224-226 Well-connected electron density is observed for the glycine flap region (71-76), presumable due to strong interactions with AMP-PNP. The adenine ring of AMP-PNP forms hydrogen bonding interactions with the peptide backbone of residues Glu 139 and Leu 141. In addition, the bicyclic ring of adenine forms close contacts with residues, Ala 91, Met 138, Cys 140, Val 118, Leu 70, and Val 78. The ribose sugar of AMP-PNP interacts with residues, Gly 71, Leu 72, Glu 145, and Leu 193. The triphosphate moiety is surrounded by amino acid residues, Leu 72, Gly 73, Ile 74, Asn 75, Val 78, Asp 207, Lys 93, Lys 188, Asn 191, Glu 190 and Thr 206. The auto-inhibitory domain of MK-2 folds back on the protein and approaches the binding sites for ATP and the peptide substrate. As a result, the ATP binding site is constricted even further.

What is claimed is:

1. Crystalline MK-2.

2. The crystalline MK-2 of claim 1 wherein said MK-2 is human MK-2.

3. The crystalline MK-2 of claim 1 wherein said MK-2 is crystallized with a crystallization additive selected from the group consisting of cobaltous chloride hexahydrate, magnesium chloride, strontium chloride hexahydrate, yttrium chloride hexahydrate, ethanol, methanol, trimethylamine hydrochloride, urea, EDTA sodium salt, NAD⁺, D(+) flucose, spermidine, spermidine-tetra-HCl, glycine, glycyl-glycyl-glycine, dimethyl sulfoxide, sodium fluoride, tert-butanol, 1,3-propanediol, n-propanol, acetone, dichloromethane, 1,4-dithio-DL-threitol, C₁₂E₈, n-dodecyl-D-maltoside, TRITON X-100, deoxy-BigChap, Anapoe® X-114, Anapoe® C₁₃E₈, C-HEGA-8™, n-hexadecyl-D-maltoside, n-tetradecyl-D maltoside, n-tridecyl-D maltoside, FOS-Choline® 9, and Cymal® −1.

4. The crystalline MK-2 of claim 1 wherein said MK-2 is crystallized with a crystallization additive selected from the group consisting of deoxy-BigChap, n-hexadecyl-beta-D-maltoside, Yttrium chloride hexahydrate, and n-tridecyl-beta-D-maltoside.

5. The crystalline MK-2 of claim 1 wherein said MK-2 is crystallized with deoxy-BigChap.

6. The crystalline MK-2 of claim 1 wherein said MK-2 is crystallized with n-hexadecyl-beta-D-maltoside.

7. The crystalline MK-2 of claim 1 wherein said MK-2 is crystallized with Yttrium chloride hexahydrate.

8. The crystalline MK-2 of claim 1 wherein said MK-2 is crystallized with n-tridecyl-b eta-D-maltoside.

9. A human MK-2 construct comprising SEQ ID No. 1.

10. A human MK-2 construct comprising SEQ ID No. 1 and conservative substitutions thereof.

11. A crystalline composition comprising MK-2 in association with an additional species in a co-complex.

12. The crystalline composition of claim 11 wherein the additional species comprises an ATP analogue.

13. The crystalline composition of claim 12 wherein said ATP analogue is AMP-PNP.

14. A composition which comprises MK-2 polypeptide molecules arranged in a crystalline manner in a space group F4₁32, so as to form a unit cell of dimensions a=b=c= about 252 to about 256 angstroms, which effectively diffracts X-rays for determination of atomic coordinates of the MK-2 polypeptide to a resolution of better than 3.5 angstroms.

15. A composition which comprises MK-2 polypeptide molecules arranged in a crystalline manner in a space group F4₁32, so as to form a unit cell of dimensions a=b=c= about 254.8 angstroms, which effectively diffracts X-rays for determination of atomic coordinates of the MK-2 polypeptide to a resolution of between about 2.5 to about 3.3 angstroms.

16. A model of the structure of MK-2 comprising a data set embodying the structure of the crystalline MK-2 of claim 1.

17. The model of claim 16 wherein said data set was determined by crystallographic analysis of MK-2.

18. The model of claim 16 wherein said data set embodies the entire structure of MK-2.

19. The model of claim 16 wherein said data set embodies a portion of the structure of MK-2.

20. The model of claim 19 wherein said portion is the ATP binding site of MK-2.

21. The model of claim 16 wherein said MK-2 exists in a co-complex with an ATP analogue.

22. The model of claim 21 wherein said ATP analogue is AMP-PNP.

23. A computer readable medium having stored thereon the model of claim 16.

24. A method of identifying a species which is an inhibitor of MK-2 activity comprising:
(a) providing the model of claim 16;
(b) studying the interaction of candidate species with such model; and
(c) selecting a species which is predicted to act as said inhibitor.

25. A species identified in accordance with the method of claim 24.

26. A method of growing crystals comprising:
(a) providing a solution of MK-2 polypeptide molecules;
(b) providing a precipitant solution comprising about 1.6 to about 2.6M ammonium sulfate, about 80-120 mM sodium acetate and about 2-50 mM of a crystallization additive; and
(c) combining the solution of MK-2 polypeptide molecules with the precipitant solution and allowing crystals of protein MK-2 to form.

27. The method of claim 26 wherein the crystals of protein MK-2 are formed in the presence of Mg²⁺, an ATP analogue and an inhibitor.

28. The method of claim 27 wherein the ATP analogue is AMP-PNP.

29. The method of claim 27 wherein the inhibitor is SC-83598.

30. The method of claim 26 wherein the crystallization additives are selected from the group consisting of cobaltous chloride hexahydrate, magnesium chloride, strontium chloride hexahydrate, yttrium chloride hexahydrate, ethanol, methanol, trimethylamine hydrochloride, urea, EDTA sodium salt, NAD⁺, D(+) flucose, spermidine, spermidine-tetra-HCl, glycine, glycyl-glycyl-glycine, dimethyl sulfoxide, sodium fluoride, tert-butanol, 1,3-propanediol, n-propanol, acetone, dichloromethane, 1,4-dithio-DL-threitol, C₁₂E₈, n-dodecyl-D-maltoside, TRITON X-100, deoxy-BigChap, Anapoe® X-114, Anapoe® C₁₃E₈, C-HEGA-8™, n-hexadecyl-D-maltoside, n-tetradecyl-D maltoside, n-tridecyl-D maltoside, FOS-Choline® 9, and Cymal −1.

31. The method of claim 26 wherein the crystallization additive comprises deoxy-BigChap, n-hexadecyl-beta-D-maltoside, Yttrium chloride hexahydrate, and n-tridecyl-beta-D-maltoside.

32. The method of claim 30 wherein the crystallization additives are present in a concentration of between about 10 to about 20 mM.

33. A method of crystallizing MK-2 wherein X-rays taken of the resulting crystal can be diffracted to a resolution of 3.5 angstroms or better.

34. The method of claim 26 wherein the X-rays taken of the resulting crystal can be diffracted to a resolution of between about 2.5 to about 3.3 angstroms.

35. A method of solving a crystal structure, the method comprising using the structure coordinates of the crystal of claim 1, or portions thereof, to solve a crystal form of a mutant, homologue, or co-complex of MK-2.

36. A method for determining the three-dimensional structure of the crystallized MK-2 protein comprising the data set of claim 16, having space group F4₁32, and a resolution of about 3.0 angstroms, the method comprising:
(a) crystallizing the MK-2 protein from a solution containing a crystallization additive; and
(b) analyzing a crystal to determine the three-dimensional structure.

37. The method of claim 36 wherein the crystallization additive is selected from the group consisting of cobaltous chloride hexahydrate, magnesium chloride, strontium chloride hexahydrate, yttrium chloride hexahydrate, ethanol, methanol, trimethylamine hydrochloride, urea, EDTA sodium salt, NAD⁺, D(+) flucose, spermidine, spermidine-tetra-HCl, glycine, glycyl-glycyl-glycine, dimethyl sulfoxide, sodium fluoride, tert-butanol, 1,3-propanediol, n-propanol, acetone, dichloromethane, 1,4-dithio-DL-threitol, C₁₂E₈, n-dodecyl-D-maltoside, TRITON X-100, deoxy-BigChap, Anapoe® X-114, Anapoe® C₁₃E₈, C-HEGA-8™, n-hexadecyl-D-maltoside, n-tetradecyl-D maltoside, n-tridecyl-D maltoside, FOS-Choline® 9, and Cymal −1.

38. The method of claim 29 wherein the crystallization additive is selected from the group consisting of deoxy-BigChap, n-hexadecyl-beta-D-maltoside, Yttrium chloride hexahydrate, and n-tridecyl-beta-D-maltoside.

39. A method of identifying inhibitors of MK-2 by rational drug design comprising:
(a) designing a potential inhibitor that will bond with one or more amino acids in the ATP binding sequence selected from the group consisting of amino acid residues 69-80,90-95, 104, 108, 118-119, 136-147, 184-195, 204-210, and 224-226 based upon the crystal structure co-ordinates of crystalline of MK-2 of claim 1;
(b) synthesizing the inhibitor; and
(c) determining whether the potential inhibitor inhibits the activity of MK-2.

40. The method of claim 39 wherein said inhibitor is designed to interact with one or more amino acids in the sequence selected from the group consisting of Gly 7 1, Leu 72, Gly 73, Ile 74, Asn 75, and Gly 76.

41. The method of claim 39 wherein said inhibitor is designed to interact with one or more amino acids selected from the group consisting of Glu 139 and Leu 141.

42. The method of claim 39 wherein said inhibitor is designed to interact with one or more amino acids in the sequence selected from the group consisting of Ala 91, Met 138, Cys 140, Val 118, Leu 70, and Val 78.

43. The method of claim 39 wherein said inhibitor is designed to interact with one or more amino acids in the sequence selected from the group consisting of Gly 71, Leu 72, Glu 145, and Leu 193.

44. The method of claim 39 wherein said inhibitor is designed to interact with one or more amino acids in the sequence selected from the group consisting of Leu 72, Gly 73, Ile 74, Asn 75, Val 78, Asp 207, Lys 93, Lys 188, Asn 191, Glu 190 and Thr 206.

45. A computer-assisted method for identifying an inhibitor of MK-2 activity comprising:
(a) supplying a computer modeling application with a set of structure coordinates of a molecule or molecular complex comprising at least a portion of an MK-2 or MK-2-like ATP binding site, the ATP binding site comprising amino acids 69-80, 90-95, 104, 108, 118-119, 136-147, 184-195,204-210,and224-226;
(b) supplying the computer modeling application with a set of structure coordinates of a chemical entity; and
(c) determining whether the chemical entity is an inhibitor expected to bind to or interfere with the molecule or molecular complex.

46. A computer-assisted method for designing an inhibitor of MK-2 activity comprising:
(a) supplying a computer modeling application with a set of structure coordinates of a molecule or molecular complex comprising at least a portion of an MK-2 or MK-2-like ATP binding site, the ATP binding site comprising amino acids 69-80, 90-95, 104, 108, 118-119, 136-147, 184-195,204-210,and 224-226;
(b) supplying the computer modeling application with a set of structure coordinates of a chemical entity; and
(c) evaluating the potential binding interactions between the chemical entity and ATP binding site of the molecule or molecular complex;
(d) structurally modifying the chemical entity to yield a set of structure coordinates for a modified chemical entity; and
(e) determining whether the modified chemical entity is an inhibitor.

47. A computer-assisted method for designing an inhibitor of MK-2 activity de novo comprising:
(a) supplying a computer modeling application with a set of structure coordinates of a molecule or molecular complex comprising at least a portion of an MK-2 or MK-2-like ATP binding site, the ATP binding site comprising amino acids 69-80, 90-95, 104, 108, 118-119, 136-147, 184-195,204-210, and 224-226;
(b) computationally building a chemical entity represented by a set of structure coordinates; and
(c) determining whether the chemical entity is an inhibitor expected to bind to or interfere with the molecule or molecular complex, wherein binding to or interfering with the molecule or molecular complex is indicative of potential inhibition of MK-2 activity.


SEQUENCE LISTING (2 sequences)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 2 - protein, 327 aa, Homo sapiens
QFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIK